(19) United States
(12) Patent Application Publication
(10) Pub. No.: US 2020/0095307 A1
(43) Pub. Date: Mar. 26, 2020

(54) COMPOSITIONS AND METHODS FOR THE TREATMENT OF NEUROMYELITIS OPTICA

(71) Applicant: Alexion Pharmaceuticals, Inc., Boston, MA (US)

(72) Inventor: Fanny O'Brien, Norwell, MA (US)

(21) Appl. No.: 16/577,703

(22) Filed: Sep. 20, 2019

Related U.S. Application Data
(60) Provisional application No. 62/884,642, filed on Aug. 8, 2019.
(60) Provisional application No. 62/734,865, filed on Sep. 21, 2018.

(51) Int. Cl. C07K 16/18; A61P 37/06

(52) U.S. Cl. CPC C07K 16/18; A61K 31/52; A61P 37/06

(57) ABSTRACT

Provided are methods of treating neuromyelitis optica spectrum disorder (NMOSD) in a subject in need thereof by administering to the subject a substance that specifically binds complement component 5 (C5). In some embodiments, the substance that specifically binds C5 is a binding protein, e.g., an anti-C5 antibody.

### RELATED APPLICATIONS

This application claims the benefit of priority to U.S. Provisional Patent Application Ser. No. 62/734,865, filed Sep. 21, 2018, and U.S. Provisional Patent Application Ser. No. 62/884,642, filed Aug. 8, 2019, the entire contents of which are incorporated herein by reference for all purposes.

### SEQUENCE LISTING

The instant application contains a sequence listing which has been submitted electronically in ASCII format and is hereby incorporated by reference in its entirety. Said ASCII copy, created on Sep. 20, 2019, is named “616734_AX9-001_SEQ_LIST_ST25” and is 34,039 bytes in size.

### BACKGROUND

Neuromyelitis optica (NMO), also known as Devic's Disease, or Devic's Syndrome, and, more broadly, part of neuromyelitis optica spectrum disorder (NMOSD), is a rare, severe disabling autoimmune inflammatory disorder of the central nervous system (CNS) that predominately affects the optic nerves and spinal cord, often leading to blindness, mono/para/tetrapalegia, and respiratory failure. NMOSD is characterized by a relapsing disease course, from which recovery may be poor due to the stepwise accumulation of significant neurologic disability.

The clinical hallmarks of NMO are acute optic neuritis and transverse myelitis that frequently involves greater than three vertebral levels, described as longitudinally extensive transverse myelitis (LETM). These clinical events can occur either simultaneously or in isolation. Signs and symptoms attributable to lesions beyond the optic nerves and spinal cord can also occur in patients with NMO, and are reported in about 15% of patients. The clinical presentation of NMO can be quite variable and may elude diagnosis at the time of the first attack or even the second attack.

Aquaporin-4 (AQP4) is a water channel protein expressed in the CNS, mainly by astrocytes. AQP4 immunoglobulin G (IgG), an antibody present in 65-88% of patients with NMOSD, is the first ever biomarker specific to an inflammatory, demyelinating CNS disorder. Preclinical data indicate that AQP4-IgG triggers the complement cascade, leading to inflammation and formation of the complement-mediated membrane attack complex (MAC). AQP4-IgG-triggered MAC has been implicated in astrocyte destruction and bystander neuronal injury, but is not seen in the presence of a complement inhibitor. With the discovery of NMO-IgG, the diagnostic criteria for NMO were revised in 2006 to include the testing of this disease-specific antibody.

In light of the fact that NMO is a disorder that has the potential to cause significant disability, the ability to recognize and differentiate NMO and related disorders from other demyelinating disorders is important from a clinical perspective. The prognosis of relapsing NMO is poor. The 5-year mortality of NMO was reported to be 30%; 50% sustain permanent severe disability, visual (blind in one or both eyes) or ambulatory (requiring a wheelchair). Most deaths result from neurogenic respiratory failure secondary to a high cervical cord or brainstem lesion. Frequent early relapses predict a poor prognosis. Relapse prevention is thus the primary therapeutic imperative.

Currently, there is no therapy approved for the treatment of NMO or relapse prevention. Additionally, there have been no randomized placebo controlled trials examining therapeutic approaches for treatment of NMO. Standard treatment options including steroids and other immunosuppressive agents as supportive treatments are used based on clinical experience and consensus. Acute NMO relapses are generally treated with high-dose IV steroids with plasma exchange (PE) often used as a rescue therapy for those who do not respond. Supportive treatments against relapse currently use broad spectrum or selective B-lymphocyte immunosuppressants. Recently, Eculizumab was proved to reduce relapse frequency in an open-label study in 14 patients with AQP4-IgG-positive disease.

Of the immunosuppressive agents, corticosteroid, AZA, mycophenolate mofetile and rituximab are probably most commonly used for long-term prophylaxis. Depending on regional medical options, the supportive medications option for NMO may vary. In the US, options include corticosteroids, AZA, MMF, rituximab, and mitoxantrone, whereas corticosteroids including oral prednisone or pulse-high dose steroids (IV) are common treatments in Japan. A significant number of patients (>50%) will continue to have attacks resulting in additional and permanent neurologic deficits and disability. Given the seriousness of the disease, limitations of currently available therapies, and the limited options for treatment, there remains a significant unmet medical need for an effective and safe treatment for NMO.

### SUMMARY

The disclosure provides methods of treating neuromyelitis optica (NMO) and neuromyelitis optica spectrum disorder (NMOSD) in subjects in need thereof by administering an antibody that specifically binds complement component 5 (C5). In some embodiments, the antibody is eculizumab. In some embodiments, the antibody that specifically binds C5 reduces the rate at which C5 is cleaved, in vivo, into C5a and C5b. In some embodiments, the antibody that specifically binds C5, binds to one or both of the C5a and/or C5b fragments. In any of these embodiments, the antibody that specifically binds C5 reduces the complement cascade at C5, thereby reducing the release of proinflammatory mediators and the formation of a cytolytic pore.

In some embodiments, the subject has a time to relapse greater than 6 months during administration of eculizumab. In some embodiments, the subject shows a reduction in annualized relapse rate (ARR) relative to baseline during administration of eculizumab. In some embodiments, the subject shows a reduction in expanded disability scale score (EDSS) relative to baseline during administration of eculizumab. In some embodiments, the subject shows an increase in quality of life (EQ-5D) score relative to baseline during administration of eculizumab. In some embodiments, the subject shows a reduction in Hauser ambulatory index (HAI) relative to baseline during administration of eculizumab.

Empirical data indicate that serum eculizumab concentrations greater than 50 μg/mL and closer to at least 100 μg/mL are required to significantly reduce free C5 concentrations. Specifically, free C5 concentration was reduced significantly with increasing concentrations of eculizumab beginning at >50 μg/mL and was at near zero levels with eculizumab concentrations above 100 μg/mL. Thus, In some embodiments, the method comprises administering a therapeutically effective amount of eculizumab to the subject, wherein the therapeutically effective amount of eculizumab is at maintained at a concentration of least 50 μg/mL of eculizumab in serum of the subject. In some embodiments, the method comprises administering a therapeutically effective amount of eculizumab to the subject, wherein the therapeutically effective amount of eculizumab is at maintained at a concentration of least 60 μg/mL of eculizumab in serum of the subject. In one embodiment, the method comprises administering a therapeutically effective amount of eculizumab to the subject, wherein the therapeutically effective amount of eculizumab is at maintained at a concentration of least 70 μg/mL of eculizumab in serum of the subject. In some embodiments, the method comprises administering a therapeutically effective amount of eculizumab to the subject, wherein the therapeutically effective amount of eculizumab is at maintained at a concentration of least 80 μg/mL of eculizumab in serum of the subject. In some embodiments, the method comprises administering a therapeutically effective amount of eculizumab to the subject, wherein the therapeutically effective amount of eculizumab is at maintained at a concentration of least 90 μg/mL of eculizumab in serum of the subject. In some embodiments, the method comprises administering a therapeutically effective amount of eculizumab to the subject, wherein the therapeutically effective amount of eculizumab is at maintained at a concentration of least 100 μg/mL of eculizumab in serum of the subject.

In some embodiments, the method comprises administering a therapeutically effective amount of eculizumab to the subject, wherein the therapeutically effective amount of eculizumab is at maintained at a concentration of between 50-100 μg/mL, between 60-100 μg/mL, between 70-100 μg/mL, between 80-100 μg/mL or between 90-100 μg/mL of eculizumab in serum of the subject.

In some embodiments, the method comprises selecting a human subject in need of treatment and administering at least 900 mg of eculizumab to the human subject, thereby treating NMOSD in the subject.

Accordingly, an aspect of the disclosure provides a method for treating a neuromyelitis optica spectrum disorder (NMOSD) comprising: selecting a human subject in need thereof; and administering a therapeutically effective amount of eculizumab or an antigen binding fragment thereof to the human subject, thereby treating NMOSD in the subject in need thereof.

In some embodiments, the subject is selected as having three or more relapses in the past 24 months, or at least two or more relapses in the last 12 months. In some embodiments, the subject having three or more relapses in the past 24 months experienced on more relapses in the past 12 months.

In some embodiments, the method comprises selecting a human subject in need of treatment and administering a therapeutically effective amount of eculizumab to the human subject, thereby treating NMOSD in the subject, wherein the subject is selected as having an annualized relapse rate (ARR) of greater than 1.0 in the past 24 months. In some embodiments, the subject is selected as having an ARR of greater than 1.0 in the past 12 months.

In some embodiments, the method comprises selecting a human subject in need of treatment and administering a therapeutically effective amount of eculizumab to the human subject, thereby treating NMOSD in the subject, wherein the subject is selected as having an Expanded Disability Status Scale (EDSS) score of 1.0 or greater in the past 24 months.

In some embodiments, the method comprises selecting a human subject in need of treatment and administering a therapeutically effective amount of eculizumab to the human subject, thereby treating NMOSD in the subject, wherein the subject is selected as having EDSS score of 1.0 or greater in the past 24 months, wherein the subject is selected as having an EDSS score greater than 2.5 and less than 7 in the 24 months before administration of eculizumab.

In some embodiments, the method comprises selecting a human subject in need of treatment and administering a therapeutically effective amount of eculizumab to the human subject, thereby treating NMOSD in the subject, wherein the subject is selected as having a Hauser ambulation index (HAI) score of 0.0 or greater in the past 24 months.

In some embodiments, the method comprises selecting a human subject in need of treatment and administering a therapeutically effective amount of eculizumab to the human subject, thereby treating NMOSD in the subject, wherein the subject is selected as having a modified Rankin Scale (mRS) score of 0.0 to 2.5 in the past 24 months.

In some embodiments, the method comprises selecting a human subject in need of treatment and administering a therapeutically effective amount of eculizumab to the human subject, thereby treating NMOSD in the subject, wherein eculizumab is administered without additional immunosuppressive therapies (ISTs).

In some embodiments, the method comprises selecting a human subject in need of treatment and administering a therapeutically effective amount of eculizumab to the human subject, thereby treating NMOSD in the subject, wherein eculizumab is administered with at least one IST.

In some embodiments, the method comprises selecting a human subject in need of treatment and administering a therapeutically effective amount of eculizumab to the human subject, thereby treating NMOSD in the subject, wherein eculizumab is administered with at least one IST, wherein the at least one IST is selected from the group consisting of a steroid, azathioprine (AZA), and mycophenolate mofetil (MMF).

In some embodiments, the method comprises selecting a human subject in need of treatment and administering a therapeutically effective amount of eculizumab to the human subject, thereby treating NMOSD in the subject, wherein eculizumab is administered with a steroid and at least one additional IST.

In some embodiments, the method comprises selecting a human subject in need of treatment and administering a therapeutically effective amount of eculizumab to the human subject, thereby treating NMO in the subject, wherein eculizumab is administered with a steroid and at least one additional IST, wherein the at least one IST is AZA or MMF.

In some embodiments, the method comprises treating NMOSD wherein the subject experiences a greater than 80% risk reduction for relapse.

In some embodiments, the method comprises treating NMOSD wherein the subject experiences a greater than 90% risk reduction for relapse.

In some embodiments, the method comprises treating NMOSD wherein the subject has a time to relapse greater than 6 months during administration of eculizumab.

In some embodiments, the method comprises treating NMOSD wherein the subject has a time to relapse greater than 48 weeks during administration of eculizumab.

In some embodiments, the method comprises treating NMOSD wherein the subject has a time to relapse greater than 24 months during administration of eculizumab.

In some embodiments, the method comprises treating NMOSD wherein the subject is selected for being 18 years or more in age.

In some embodiments, the method comprises treating NMOSD wherein the subject is selected for being a male.

In some embodiments, the method comprises treating NMOSD wherein between 900 and 1600 mg of eculizumab is administered to the subject.

In some embodiments, the method comprises treating NMOSD wherein between 1000 and 1400 mg of eculizumab is administered to the subject.

In some embodiments, the method comprises treating NMOSD wherein eculizumab is administered to the subject once a month, once every two weeks, once a week, twice a week or three times a week.

In some embodiments, the method comprises treating NMOSD wherein eculizumab is administered once a week or once every two weeks.

In some embodiments, the method comprises treating NMOSD wherein eculizumab is administered once a week.

In some embodiments, the method comprises treating NMOSD wherein eculizumab is administered once a week, wherein between 900 and 1600 mg of eculizumab is administered to the subject.

In some embodiments, the method comprises treating NMOSD wherein eculizumab is administered once a week, wherein between 900 and 1400 mg of eculizumab is administered to the subject.

In some embodiments, the method comprises treating NMOSD wherein eculizumab is administered once a week, wherein between 1000 and 1300 mg of eculizumab is administered to the subject.

In some embodiments, the method comprises treating NMOSD wherein eculizumab is administered once a week, wherein all weekly doses are approximately the same.

In some embodiments, the method comprises treating NMOSD wherein weekly doses are different.

In some embodiments, the method comprises treating NMOSD wherein eculizumab is administered once a week, wherein weekly doses are different, wherein the doses are between 900 and 1300 mg of eculizumab or between 1000 and 1600 mg of eculizumab to the subject.

In some embodiments, the method comprises treating NMOSD wherein eculizumab is administered in a multiphase dosing regimen.

In some embodiments, the method comprises treating NMOSD wherein eculizumab is administered in a multiphase dosing regime, wherein the multiphase dosing regimen comprises a first phase and a second phase.

In some embodiments, the method comprises treating NMOSD wherein eculizumab is administered in a multiphase dosing regime, wherein the multiphase dosing regimen comprises a first phase and a second phase, wherein the first phase comprises administration of eculizumab at between 900 and between 1600 mg once a week to the subject for between 3-10 weeks.

In some embodiments, the method comprises treating NMOSD wherein eculizumab is administered in a multiphase dosing regime, wherein the multiphase dosing regimen comprises a first phase and a second phase, wherein the first phase comprises administration of eculizumab at between 900 and between 1600 mg once a week to the subject for between 3-10 weeks, wherein the first phase comprises administration of eculizumab at between 1000 and 1400 mg once a week to the subject for between 4-6 weeks.

In some embodiments, the method comprises treating NMOSD wherein eculizumab is administered in a multiphase dosing regime, wherein the multiphase dosing regimen comprises a first phase and a second phase, wherein the first phase comprises administration of eculizumab at about 1000 mg once a week for 4 weeks and at about 1300 mg for one week to the subject.

In some embodiments, the method comprises treating NMOSD wherein eculizumab is administered in a multiphase dosing regime, wherein the second phase comprises administration of eculizumab at between 900 and between 1600 mg once every other week to the subject for between 3-30 weeks.

In some embodiments, the method comprises treating NMOSD wherein eculizumab is administered in a multiphase dosing regime, wherein the second phase comprises administration of eculizumab at between 900 and between 1600 mg once every other week to the subject for between 3-30 weeks, wherein the second phase comprises administration of eculizumab at between 1000 and 1400 mg once every other week to the subject for between 4-25 weeks.

In some embodiments, the method comprises treating NMOSD wherein eculizumab is administered in a multiphase dosing regime, wherein the second phase comprises administration of eculizumab at between 900 and between 1600 mg once every other week to the subject for between 3-30 weeks, wherein the second phase comprises administration of eculizumab at between 1000 and 1400 mg once every other week to the subject for between 4-25 weeks, wherein the second phase comprises administration of eculizumab at about 1200 mg once every other week to the subject for 20 weeks.

In some embodiments, the method comprises treating NMOSD wherein eculizumab is administered in a multiphase dosing regime, wherein the multiphase dosing regimen comprises a first phase and a second phase, wherein the multiphase dosing regimen further comprises a third phase.

In some embodiments, the method comprises treating NMOSD wherein eculizumab is administered in a multiphase dosing regime, wherein the multiphase dosing regimen comprises a first phase and a second phase, wherein the multiphase dosing regimen further comprises a third phase, wherein the third phase comprises the performance of plasmapheresis on the subject and administration of eculizumab at greater than 400 and less than 1200 mg to the subject within 2 hours of the completion of plasmapheresis.

In some embodiments, the method comprises treating NMOSD wherein eculizumab is administered in a multiphase dosing regime, wherein the multiphase dosing regimen comprises a first phase and a second phase, wherein the multiphase dosing regimen further comprises a third phase, wherein the third phase comprises the performance of plasmapheresis on the subject and administration of eculizumab at greater than 400 and less than 1200 mg to the subject within 2 hours of the completion of plasmapheresis, wherein the third phase comprises the performance of plasmapheresis on the subject and administration of eculizumab at greater than 500 and less than 800 mg to the subject within 90 minutes of the completion of plasmapheresis.

In some embodiments, the method comprises treating NMOSD wherein eculizumab is administered in a multiphase dosing regime, wherein the multiphase dosing regimen comprises a first phase and a second phase, wherein the multiphase dosing regimen further comprises a third phase, wherein the third phase comprises the performance of plasmapheresis on the subject and administration of eculizumab at greater than 400 and less than 1200 mg to the subject within 2 hours of the completion of plasmapheresis, wherein the third phase comprises the performance of plasmapheresis on the subject and administration of eculizumab at greater than 500 and less than 800 mg to the subject within 90 minutes of the completion of plasmapheresis, wherein the third phase comprises the performance of plasmapheresis on the subject and administration of eculizumab at about 600 mg to the subject within 60 minutes of the completion of plasmapheresis.

Based on the free C5 PK/PD model, mean Cmax, and Cmin values of eculizumab during the Induction Phase (900 mg dose weekly) would result in 92.9% and 91.8% inhibition of free C5, respectively. Similarly, mean Cmax and Cmin values of eculizumab during the Maintenance Phase (1200 every 14 days) would result in 93.4% and 92.8% inhibition of free C5, respectively.

In some embodiments, the method comprises administering to the subject at least 900 mg of eculizumab. In some embodiments, the method comprises administering between 900 and 1600 mg of eculizumab to the subject. In some embodiments, the method comprises administering between 1000 and 1600 mg of eculizumab to the subject.

In some embodiments, eculizumab is administered to the subject once a month, once every two weeks, once a week, twice a week or three times a week. In some embodiments, eculizumab is administered once a week or once every two weeks. In some embodiments, eculizumab is administered once a week.

In some embodiments, between 900 and 1600 mg of eculizumab is administered once a week to the subject. In some embodiments, between 900 and 1400 mg of eculizumab is administered once a week to the subject. In some embodiments, between 1000 and 1300 mg of eculizumab is administered once a week to the subject.

In some embodiments, the weekly doses are approximately the same. In some embodiments, the weekly doses are different. In some embodiments in which the weekly doses are different, between 900 and 1300 mg of eculizumab or between 1000 and 1600 mg of eculizumab is administered to the subject.

In some embodiments, eculizumab is administered in a multiphase dosing regimen. For example, the multiphase dosing regimen comprises a first phase and a second phase In some embodiments. In some embodiments, the first phase comprises administration of eculizumab at between 900 and 1600 mg once a week to the subject for between 3-10 weeks. In some embodiments, the first phase comprises administration of eculizumab at between 1000 and 1400 mg once a week to the subject for between 4-6 weeks. In some embodiments, the first phase comprises administration of eculizumab at about 1000 mg once a week for 4 weeks and at about 1300 mg for one week to the subject.

In some embodiments, the second phase comprises administration of eculizumab at between 900 and 1600 mg once every other week to the subject for between 3-30 weeks. In some embodiments, the second phase comprises administration of eculizumab at between 1000 and 1400 mg once every other week to the subject for between 4-25 weeks. In some embodiments, the second phase comprises administration of eculizumab at about 1200 mg once every other week to the subject for 20 weeks.

In some embodiments, the multiphase dosing regimen further comprises a third phase. In some embodiments, the third phase comprises the performance of plasmapheresis on the subject and administration of eculizumab at between 400 and 1200 mg to the subject within 2 hours of the completion of plasmapheresis. In some embodiments, the third phase comprises the performance of plasmapheresis on the subject and administration of eculizumab at between 500 and 800 mg to the subject within 90 minutes of the completion of plasmapheresis. In some embodiments, the third phase comprises the performance of plasmapheresis on the subject and administration of eculizumab at about 600 mg to the subject within 60 minutes of the completion of plasmapheresis.

In some embodiments, the anti-C5 antibody or an antigen binding fragment thereof is selected from the group consisting of eculizumab, 7086 antibody, 8110 antibody, 305LO5, and SKY59.

In some embodiments, a patient switches from receiving one C5 inhibitor to a different C5 inhibitor during the course of treatment. In some embodiments, different anti-C5 antibodies may be administered during separate treatment periods.

Another aspect of the disclosure provides a method for treating a neuromyelitis optica NMOSD in a human subject in need thereof comprising administering a therapeutically effective amount of eculizumab to the human subject, wherein the human subject is positive for auto-antibodies binding aquaporin-4 (NMO-IgG) and shows one or more signs or symptoms of NMSOD. In some embodiments, the human subject is 18 years of age or older. In some embodiments, the antibody is eculizumab. In some embodiments, the human subject is selected for displaying one or more symptoms of NMOSD prior to administration of eculizumab. In some embodiments, the human subject is administered eculizumab for at least 26 weeks. In some embodiments, eculizumab is administered to the human subject by intravenous infusion.

In some embodiments, the method comprises treating NMOSD in a human subject in need thereof, wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28.

In some embodiments, the method comprises treating NMOSD in a human subject in need thereof, wherein eculizumab is administered using a phased dosing schedule, wherein the 28 day induction phase of eculizumab treatment is followed by a maintenance phase comprising administering 1200 mg of eculizumab 14 days after the fifth induction dose and administering 1200 mg of eculizumab every 14±2 days thereafter.

In some embodiments, the method comprises treating NMOSD in a human subject in need thereof, wherein eculizumab is administered using a phased dosing schedule, wherein the 28 day induction phase and maintenance phase of eculizumab treatment further comprising performing plasmapheresis on the human subject and administering eculizumab at a dose of between 300 mg and 1200 mg to the human subject within 4 hours of completion of plasmapheresis.

In some embodiments, the method comprises treating NMOSD in a human subject in need thereof, wherein eculizumab is administered using a phased dosing schedule, wherein the 28 day induction phase and maintenance phase of eculizumab treatment further comprising performing plasmapheresis on the human subject and administering eculizumab at a dose of between 600 mg and 900 mg to the human subject within 90 minutes of completion of plasmapheresis.

In some embodiments, the method comprises treating NMOSD in a human subject in need thereof, wherein eculizumab is administered using a phased dosing schedule, wherein the 28 day induction phase and maintenance phase of eculizumab treatment further comprising performing plasmapheresis on the human subject and administering eculizumab at a dose of 600 mg to the human subject within 1 hour of completion of plasmapheresis.

In some embodiments, before administration of eculizumab, the human subject experienced three or more relapses in the previous 24 months or at least two relapses in the previous 12 months. In some embodiments, before administration of eculizumab, the human subject having three or more relapses in the previous 24 months experienced at least one relapse in the previous 12 months.

In some embodiments, before administration of eculizumab, the human subject experienced an ARR of greater than 1.0 in the previous 24 months.

In some embodiments, before administration of eculizumab, the human subject experienced an EDSS score of greater than 1.0 in the previous 24 months. In some embodiments, the human subject experienced an EDSS score greater than 2.5 and less than 7 in the 24 months before administration of eculizumab.

In some embodiments, the human subject experienced a HAI score of 0.0 or greater in the previous 24 months before eculizumab is administered to the human subject.

In some embodiments, before administration of eculizumab, the human subject experienced a mRS score of 0.0 to 2.5 in the previous 24 months.

In some embodiments, eculizumab is administered to the human subject without additional ISTs.

In some embodiments, eculizumab is administered to the human subject with at least one IST. In some embodiments, eculizumab is administered to the human subject with at least one IST, wherein the at least one IST is selected from the group consisting of a steroid, AZA, and MMF.

In some embodiments, eculizumab is administered to the human subject with a steroid and at least one additional IST. In some embodiments, eculizumab is administered to the human subject with a steroid and at least one additional IST, wherein the at least one additional IST is AZA or MMF.

In some embodiments, the human subject experiences a clinically meaningful improvement in one or more clinical markers for NMOSD progression after administration of eculizumab. In some embodiments, the human subject experiences a clinically meaningful improvement in one or more clinical markers for NMOSD progression after administration of eculizumab, wherein the one or more clinical markers for NMOSD progression are selected from the group consisting of ARR, EDSS, HAI, and mRS.

In some embodiments, the human subject experiences a greater than 80% risk reduction for relapse after administration of eculizumab. In some embodiments, the human subject experiences a greater than 90% risk reduction for relapse after administration of eculizumab.

In some embodiments, the human subject has a time to relapse greater than 6 months after administration of eculizumab. In some embodiments, the human subject has a time to relapse greater than 48 weeks after administration of eculizumab. In some embodiments, the human subject has a time to relapse greater than 24 months after administration of eculizumab.

Another aspect of the disclosure provides a method for treating NMOSD in a human subject in need thereof by administering a therapeutically effective amount of eculizumab to the human subject, wherein the human subject is positive for auto-antibodies binding aquaporin-4 (NMO-IgG) and shows one or more signs or symptoms of NMSOD, wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, and wherein the human subject experiences a clinically meaningful improvement in one or more clinical markers for NMOSD progression after administration of eculizumab, wherein the one or more clinical markers for NMOSD progression are selected from the group consisting of ARR, EDSS, HAI, and mRS

Another aspect of the disclosure provides a method for treating a NMOSD in a human subject in need thereof by administering a therapeutically effective amount of eculizumab to the human subject, wherein the human subject is positive for auto-antibodies binding aquaporin-4 (NMO-IgG) and shows one or more signs or symptoms of NMSOD, wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, wherein the 28 day induction phase of eculizumab treatment is followed by a maintenance phase comprising administering 1200 mg of eculizumab 14 days after the fifth induction dose and administering 1200 mg of eculizumab every 14±2 days thereafter, and wherein the human subject experiences a clinically meaningful improvement in one or more clinical markers for NMOSD progression after administration of eculizumab, wherein the one or more clinical markers for NMOSD progression are selected from the group consisting of ARR, EDSS, HAI, and mRS.

Another aspect of the disclosure provides a method for treating a NMOSD in a human subject in need thereof by administering a therapeutically effective amount of eculizumab to the human subject, wherein the human subject is positive for auto-antibodies binding aquaporin-4 (NMO-IgG) and shows one or more signs or symptoms of NMSOD, wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, wherein the 28 day induction phase of eculizumab treatment is followed by a maintenance phase comprising administering 1200 mg of eculizumab 14 days after the fifth induction dose and administering 1200 mg of eculizumab every 14±2 days thereafter, wherein plasmapheresis is performed on the human subject and administering eculizumab at a dose of 600 mg to the human subject within 1 hour of completion of plasmapheresis, and wherein the human subject experiences a clinically meaningful improvement in one or more clinical markers for NMOSD progression after administration of eculizumab, wherein the one or more clinical markers for NMOSD progression are selected from the group consisting of ARR, EDSS, HAI, and mRS.

According to the disclosure antibodies that specifically bind C5 include eculizumab and one or more fragments thereof. In some embodiments, eculizumab or one or more fragments thereof comprise at least one antigen binding site, wherein said antibody or fragment thereof specifically binds to the alpha chain of human complement component C5. In some embodiments, the antibody or fragment thereof inhibits complement activation in a human body fluid and does not specifically bind to the human complement activation product free C5a. In some embodiments, the antibody is produced by hybridoma 5G1.1 having ATCC designation HB-11625. In some embodiments, the antibody or fragment thereof competes with the antibody produced by hybridoma 5G1.1 having ATCC designation HB-11625 for specific binding to the alpha chain of human C5.

The disclosure also provides eculizuab for use in the treatment of neuromyelitis optical spectrum disorder (NMOSD) according to any of the embodiments, described above.

Further, the disclosure encompasses any of the above embodiments being used with any other of the above embodiments in any combination.

### BRIEF DESCRIPTION OF THE DRAWINGS

FIG. 1 is a schematic of the overall design of the clinical trial disclosed herein.

FIG. 2 is a schematic of the overall design of the clinical trial disclosed herein.

FIG. 3 is a schematic of the N. meningitidis vaccination schedule used in the clinical trial disclosed herein.

FIG. 4 is a schematic of the dosing schedule used in the clinical trial disclosed herein.

FIG. 5 is a schematic of the survey of health status used in the clinical trial disclosed herein.

FIG. 6 is a schematic of the health-related quality of life evaluative instrument used in the clinical trial disclosed herein.

FIG. 7 is a Kaplan-Meier Survival Estimate for Time to First Adjudicated On-Trial Relapse—Full Analysis Set.

FIG. 8 is a Kaplan-Meier Survival Estimate for Time to First On-Trial Relapse as Determined by the Treating Physician—Full Analysis Set,

FIG. 9 is a visual representation of Sensitivity Analyses for Time to First Adjudicated On-Trial Relapse and On-Trial Relapse of the Primary Efficacy Endpoint.

FIG. 10 is a visual representation of Subgroup Analyses on Time to First Adjudicated On-Trial Relapse.

FIG. 11 a visual representation of Subgroup Analyses on Time to First On-Trial Relapse as Determined by Treating Physician.

FIG. 12 is the formula for Annualized Relapse Rate used herein.

FIG. 13 a visual representation of the Distribution of the Change from Baseline to End of Study in EDSS Score.

FIG. 14 a visual representation of a repeated measure analysis of EDSS Score over time.

FIG. 15 a visual representation of the Distribution of the Change from Baseline to End of Study in mRS Score.

FIG. 16 a visual representation of a repeated measure analysis of MRS Score over Time.

FIG. 17 a visual representation of the Distribution of the Change from Baseline to the End of Study in HAI Score.

FIG. 18 is a visual representation of a repeated measure analysis of HAI Score over Time.

FIG. 19 is a visual representation of the Distribution of the Change from Baseline to the End of Study in EQ-5D VAS Score.

FIG. 20 is a visual representation of a repeated measure analysis of MRS Score over Time.

FIG. 21 is a visual representation of the Distribution of the Change from Baseline to the End of Study in EQ-5D Index Score.

FIG. 22 is a visual representation of a repeated measure analysis of EQ-5D Index Score over Time.

FIG. 23 is Kaplan-Meier Survival Estimate for Time to First On-Trial Relapse as Determined by the Treating Physician by Baseline IST Use: NO IST.

FIG. 24 is a schematic of the modified Rankin Scale (mRS) used in the clinical trial disclosed herein.

FIG. 25 is a schematic of the dosing, clinical evaluation and safety follow-up schedule, used in the clinical trial disclosed herein.

FIG. 26 is a Kaplan-Meier Survival Estimate for Time to First On-Trial Relapse in Patients receiving Eculizumab Plus Concomitant Supportive IST.

FIG. 27 is a box plot showing the change in physician-determined ARR of the OLE study.

FIG. 28 is a diagram summarizing the patient disposition and results of the clinical trial disclosed herein.

### DETAILED DESCRIPTION

The disclosure provides methods of treating neuromyelitis optica spectrum disorder (NMOSD) in subjects in need thereof by administering an antibody that specifically binds complement component 5 (C5). In some embodiments, the antibody that specifically binds C5 reduces the rate at which C5 is cleaved, in vivo, into C5a and C5b. In some embodiments, the antibody that specifically binds C5 binds to one or both of the C5a and/or C5b fragments. In any of these embodiments, the antibody that specifically binds C5 reduces the complement cascade at C5, thereby reducing the release of proinflammatory mediators and the formation of a cytolytic pore.

In some embodiments, the antibody that specifically binds C5 is eculizumab or a fragment thereof. According to some embodiments, eculizumab is a humanized 5G1.1 antibody (5G1.1, produced by the 5G1.1 hybridoma, ATCC designation HB-11625) with an IgG2/IgG4 chimeric constant region.

Eculizumab is a humanized monoclonal antibody (mAb) that was derived from the murine anti-human C5 antibody m5G1.1. Eculizumab specifically binds the terminal complement protein C5, thereby inhibiting its cleavage to C5a and C5b during complement activation. This strategic blockade of the complement cascade at C5 prevents the release of proinflammatory mediators and the formation of the cytolytic pore, while preserving the early components of complement activation that are essential for the opsonization of microorganisms and clearance of immune complexes.

Eculizumab is approved for the treatment of gMG, paroxysmal nocturnal hemoglobinuria, and atypical hemolytic uremic syndrome in many countries worldwide, including Japan, the USA, and countries in the European Union under the trade name Soliris®.

C5 Binding Proteins

C5 binding proteins are described in U.S. Pat. No. 6,355,245, incorporated herein by reference in its entirety. In some embodiments, the anti-C5 antibody is a monoclonal antibody having a chimeric IgG2/4 isotype. In some embodiments, the anti-C5 antibody comprises polyclonal antibodies produced and screened by conventional techniques. In some embodiments, the anti-C5 antibodies are effective in reducing the cell-lysing ability of complement present in human blood. This property of the antibodies can be determined by methods known in the art such as, for example, by the chicken erythrocyte hemolysis method described in U.S. Pat. No. 6,355,245, incorporated by reference, herein, in its entirety.

Anti C5 antibodies (or VH/VL domains derived therefrom) suitable for use herein can be identified using methods known in the art. Alternatively, art recognized anti C5 antibodies can be used. Antibodies that compete with any of these art recognized antibodies for binding to C5 also can be used.

In one embodiment, the anti-C5 antibody is Eculizumab (also known as Soliris®), comprising heavy chain CDR1, CDR2 and CDR3 domains having the sequences set forth in SEQ ID NOs:1, 2 and 3, respectively, and light chain CDR1, CDR2 and CDR3 domains having the sequences set forth in SEQ ID NOs:4, 5 and 6, respectively. Eculizumab comprises a heavy chain variable region having the amino acid sequence set forth in SEQ ID NO:7 and a light chain variable region having the amino acid sequence set forth in SEQ ID NO:8. The variable regions of eculizumab are described in PCT/US1995/005688 and U.S. Pat. No. 6,355,245, the entire teachings of which are incorporated herein by reference. Eculizumab comprises a heavy chain comprising the amino acid sequence set forth in SEQ ID NO:9 and a light chain having the amino acid sequence set forth in SEQ ID NO:10. The full heavy and light chains of eculizumab are described in PCT/US2007/006606, the entire teachings of which are hereby incorporated herein by reference.

In some embodiments, an anti C5 antibody can comprise, for example, a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering. Such variants can be combined, for example, with the CDRs or variable regions of, for example, eculizumab.

In some embodiments, an exemplary anti C5 antibody is the 7086 antibody described in U.S. Pat. Nos. 8,241,628 and 8,883,158. The anti C5 antibody can comprise, for example, the heavy and light chain CDRs or variable regions of the 7086 antibody. The anti C5 antibody can comprise, for example, comprises heavy chain CDR1, CDR2 and CDR3 domains having the sequences set forth in SEQ ID NOs: 11, 12 and 13, respectively, and light chain CDR1, CDR2 and CDR3 domains having the sequences set forth in SEQ ID NOs: 14, 15 and 16, respectively. The anti C5 antibody can comprise, for example, the VH region of the 7086 antibody having the sequence set forth in SEQ ID NO:17, and the VL region of the 7086 antibody having the sequence set forth in SEQ ID NO:18.

In some embodiments, an exemplary anti C5 antibody is the 8110 antibody also described in U.S. Pat. Nos. 8,241,628 and 8,883,158. The anti C5 antibody can comprise, for example, the heavy and light chain CDRs or variable regions of the 8110 antibody. The anti C5 antibody can comprise, for example, heavy chain CDR1, CDR2 and CDR3 domains having the sequences set forth in SEQ ID NOs: 19, 20 and 21, respectively, and light chain CDR1, CDR2 and CDR3 domains having the sequences set forth in SEQ ID NOs: 22, 23 and 24, respectively. The anti C5 antibody can comprise, for example, the VH region of the 8110 antibody having the sequence set forth in SEQ ID NO:25, and the VL region of the 8110 antibody having the sequence set forth in SEQ ID NO:26.

In some embodiments, an exemplary anti C5 antibody is the 305LO5 antibody described in US2016/0176954A1. The anti C5 antibody can comprise, for example, the heavy and light chain CDRs or variable regions of the 305LO5 antibody. The anti C5 antibody can comprise, for example, heavy chain CDR1, CDR2 and CDR3 domains having the sequences set forth in SEQ ID NOs: 27, 28 and 29, respectively, and light chain CDR1, CDR2 and CDR3 domains having the sequences set forth in SEQ ID NOs: 30, 31 and 32, respectively. In some embodiments, the antibody comprises the VH region of the 305LO5 antibody having the sequence set forth in SEQ ID NO:33, and the VL region of the 305LO5 antibody having the sequence set forth in SEQ ID NO:34.

In some embodiments, an exemplary anti C5 antibody is the SKY59 antibody (Fukuzawa, T. et al., Sci. Rep., 7:1080, 2017). The anti C5 antibody can comprise, for example, the heavy and light chain CDRs or variable regions of the SKY59 antibody. The anti C5 antibody can comprise, for example, a heavy chain comprising SEQ ID NO:35 and a light chain comprising SEQ ID NO:36.

In some embodiments, an exemplary anti C5 antibody is the REGN3918 antibody (also known as H4H12166PP) described in US20170355757. The anti C5 antibody can comprise, for example, a heavy chain variable region comprising SEQ ID NO:37 and a light chain variable region comprising SEQ ID NO:38, or a heavy chain comprising SEQ ID NO:39 and a light chain comprising SEQ ID NO:40.

The exact boundaries of CDRs have been defined differently according to different methods. In some embodiments, the positions of the CDRs or framework regions within a light or heavy chain variable domain can be as defined by Kabat et al. [(1991) “Sequences of Proteins of Immunological Interest.” NIH Publication No. 91-3242, U.S. Department of Health and Human Services, Bethesda, Md.]. In such cases, the CDRs can be referred to as “Kabat CDRs” (e.g., “Kabat LCDR2” or “Kabat HCDR1”). In some embodiments, the positions of the CDRs of a light or heavy chain variable region can be as defined by Chothia, C. et al. (Nature, 342:877 83, 1989). Accordingly, these regions can be referred to as “Chothia CDRs” (e.g., “Chothia LCDR2” or “Chothia HCDR3”). In some embodiments, the positions of the CDRs of the light and heavy chain variable regions can be as defined by a Kabat Chothia combined definition. In such embodiments, these regions can be referred to as “combined Kabat Chothia CDRs” (Thomas, T. et al., Mol. Immunol., 33:1389 401, 1996) exemplifies the identification of CDR boundaries according to Kabat and Chothia definitions.

In some embodiments, the antibody competes for binding with, and/or binds to the same epitope on C5 as, the above-mentioned antibodies (e.g., eculizumab, 7086 antibody, 8110 antibody, 305LO5 antibody, SKY59 antibody, or REGN3918 antibody). The anti C5 antibody can have, for example, at least about 90% variable region amino acid sequence identity with the above-mentioned antibodies (e.g., at least about 90%, 91%, 92%, 93%, 94%, 95%, 96%, 97%, 98% or 99% variable region identity).

An anti C5 antibody described herein can, in some embodiments, comprise a variant human Fc constant region that binds to human neonatal Fc receptor (FcRn) with greater affinity than that of the native human Fc constant region from which the variant human Fc constant region was derived. The Fc constant region can comprise, for example, one or more (e.g., two, three, four, five, six, seven, or eight or more) amino acid substitutions relative to the native human Fc constant region from which the variant human Fc constant region was derived. The substitutions, for example, can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 (while maintaining pH dependence of the interaction) are known in the art and exemplified in the working examples (PCT/US2015/019225 and U.S. Pat. No. 9,079,949 the disclosures of each of which are incorporated herein by reference in their entirety).

Substitutions that enhance the binding affinity of an antibody Fc constant region for FcRn are known in the art and include, e.g., (1) the M252Y/S254T/T256E triple substitution (Dall'Acqua, W. et al., J. Biol. Chem., 281: 23514 24, 2006); (2) the M428L or T250Q/M428L substitutions (Hinton, P. et al., J. Biol. Chem., 279:6213 6, 2004; Hinton, P. et al., J. Immunol., 176:346 56, 2006); and (3) the N434A or T307/E380A/N434A substitutions (Petkova, S. et al., Int. Immunol., 18:1759 69, 2006). Additional substitution pairings, e.g., P257I/Q311I, P257I/N434H, and D376V/N434H, have also been described (Datta-Mannan, A. et al., J. Biol. Chem., 282:1709 17, 2007). The entire teachings of each of the cited references are hereby incorporated by reference.

In some embodiments, the variant constant region has a substitution at EU amino acid residue 255 for valine. In some embodiments, the variant constant region has a substitution at EU amino acid residue 309 for asparagine. In some embodiments, the variant constant region has a substitution at EU amino acid residue 312 for isoleucine. In some embodiments, the variant constant region has a substitution at EU amino acid residue 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, nine, eight, seven, six, five, four, three or two) amino acid substitutions, insertions or deletions relative to the native constant region from which it was derived. In some embodiments, the variant Fc constant region comprises one or more amino acid substitutions selected from the group consisting of: M252Y, S254T, T256E, N434S, M428L, V259I, T250I and V308F. In some embodiments, the variant human Fc constant region comprises a methionine at position 428 and an asparagine at position 434, each in EU numbering. In some embodiments, the variant Fc constant region comprises a 428L/434S double substitution as described in, e.g., U.S. Pat. No. 8,088,376 the disclosure of which is incorporated herein by reference in its entirety.

In some embodiments the precise location of these mutations may be shifted from the native human Fc constant region position due to antibody engineering.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434 or 436 (EU numbering) relative to the native human Fc constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan or tyrosine for threonine at position 250; phenylalanine, tryptophan or tyrosine for methionine at position 252; threonine for serine at position 254; glutamic acid for arginine at position 255; aspartic acid, glutamic acid or glutamine for threonine at position 256; alanine, glycine, isoleucine, leucine, methionine, asparagine, serine, threonine or valine for proline at position 257; histidine for glutamic acid at position 258; alanine for aspartic acid at position 265; phenylalanine for aspartic acid at position 270; alanine or glutamic acid for asparagine at position 286; histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan or tyrosine for threonine at position 307; alanine, phenylalanine, isoleucine, leucine, methionine, proline, glutamine or threonine for valine at position 308; alanine, aspartic acid, glutamic acid, proline or arginine for leucine or valine at position 309; alanine, histidine or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325; valine for isoleucine at position 332; leucine for lysine at position 334; histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan or tyrosine for methionine at position 428; lysine for histidine at position 433; alanine, phenylalanine, histidine, serine, tryptophan or tyrosine for asparagine at position 434; and histidine for tyrosine or phenylalanine at position 436, all in EU numbering.

In one embodiment, the antibody binds to C5 at pH 7.4 and 25° C. (and, otherwise, under physiologic conditions) with an affinity dissociation constant (KD) that is at least 0.1 (e.g., at least 0.15, 0.175, 0.2, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95 or 0.975) nM. In some embodiments, the KD of the anti C5 antibody, or antigen binding fragment thereof, is no greater than 1 (e.g., no greater than 0.9, 0.8, 0.7, 0.6, 0.5, 0.4, 0.3 or 0.2) nM.

In some embodiments, the [(KD of the antibody for C5 at pH 6.0 at 25° C.)/(KD of the antibody for C5 at pH 7.4 at 25° C.)] is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500 or 8000).

In some embodiments, anti-C5 antibodies bind to C5 or fragments thereof, e.g., C5a or C5b. In some embodiments, the anti-C5 antibodies are immunoreactive against epitopes on the beta chain of purified human complement component C5 and are capable of blocking the conversion of C5 into C5a and C5b by C5 convertase. This ability can be measured using techniques known in the art (Wurzner, R. et al., Complement Inflamm., 8:328-40, 1991).

In some embodiments, the anti-C5 antibodies are immunoreactive against epitopes within the alpha chain of purified human complement component C5. In this embodiment the antibodies are capable of blocking the conversion of C5 into C5a and C5b by C5 convertase. In one example of this embodiment, the antibodies can provide this blockade at substantially the same concentrations needed to block hemolytic activity.

In some embodiments, the antibodies specifically bind to an amino-terminal region within the alpha chain, however, they do not specifically bind to free C5a. Hybridomas producing monoclonal antibodies reactive with complement component C5 can be obtained according to the teachings of Sims et al. (U.S. Pat. No. 5,135,916, incorporated by reference herein in its entirety). As discussed therein, antibodies are prepared using purified components of the complement membrane attack complex as immunogens. Complement component C5 or C5b can be used as the immunogen, e.g., more specifically, the alpha chain of C5.

In some embodiments, the C5 antibody is able to substantially inhibit complement hemolytic activity and to substantially inhibit the conversion of C5 to produce C5a. In some embodiments, the C5 antibodies provide these functions when used at a molar ratio of antibody to antigen (C5) of 3:1 or less.

As used herein, the term “antibodies” refers to immunoglobulins produced in vivo, as well as those produced in vitro by, for example, a hybridoma. Antibodies include, for example, antigen binding fragments (e.g., Fab′ preparations) of such immunoglobulins, as well as recombinantly expressed antigen binding proteins, including immunoglobulins, chimeric immunoglobulins, “humanized” immunoglobulins, single chain antibodies, camelid antibodies, bi-specific antibodies, and other recombinant proteins containing antigen binding domains derived from immunoglobulins.

“Specificity” refers to the ability of a binding protein to selectively bind a ligand, e.g., an antigen. The term “specifically binds,” means that a binding protein or fragment thereof forms a complex with an antigen that is relatively stable under physiologic conditions. Specific binding can be characterized by a dissociation constant of at least about 1×10⁻⁶M or less. In some embodiments, the dissociation constant is at least about 1×10⁻⁷ M, 1×10⁻⁸M or 1×10⁻⁹ M.

Methods for determining whether two molecules specifically bind are known in the art and include, for example, equilibrium dialysis, surface plasmon resonance, and the like.

### Methods of Treating Neuromyelitis Optica

The disclosure provides methods of treating subjects suffering from NMOSD by administering an antibody that specifically binds C5. As used herein, the term “subject” and “patient” are interchangeable. In some embodiments, subjects and/or patients are mammals, including, for example, primates, e.g., humans, rodents, lagomorphs, camelids, ungulates, canines and felines. In some embodiments, the subjects or patients suffering from NMOSD described herein are humans.

NMOSD is characterized by a relapsing disease course, from which recovery may be poor due to the stepwise accumulation of significant neurologic disability. Neuromyelitis optica (NMO), also known as Devic's Disease, or Devic's Syndrome is part of neuromyelitis optica spectrum disorder (NMOSD) and is a rare, severe disabling autoimmune inflammatory disorder of the central nervous system (CNS) that predominately affects the optic nerves and spinal cord, often leading to blindness, mono/para/tetrapalegia, and respiratory failure.

In some embodiments, NMO is characterized by NMO-IgG antibodies directed at aquaporin 4 (anti-AQP4). In some embodiments, a subset of NMO patients is anti-AQP4+. In some embodiments, a subset of NMO patients is anti-MOG+.

In some embodiments, AQP4 autoantibodies are found in patients with NMO-like symptoms that do not fulfill the clinical requirements to be diagnosed NMO. In some embodiments, one of the requirements to be diagnosed with NMO are recurrent and simultaneous optic nerve and spinal cord inflammation.

In some embodiments, NMOSD includes Devic's disease also known as NMO. In some embodiments NMOSD encompasses limited forms of Devic's disease, such as single or recurrent event of longitudinally extensive myelitis, and bilateral simultaneous or recurrent optic neuritis.

In some embodiments, NMOSD encompasses Asian optic-spinal MS (OSMS), or AQP4+OSMS. In some embodiments, NMOSD further encompasses longitudinally extensive myelitis or optic neuritis associated with systemic autoimmune disease, and optic neuritis or myelitis associated with lesions in specific brain areas such as the hypothalamus, periventricular nucleus, and brainstem.

In some embodiments, treatment of NMOSD includes the amelioration or improvement of one or more symptoms associated with NMOSD. Symptoms associated with NMOSD include visual impairment, decreased visual acuity, visual field defects, loss of color vision, spinal cord dysfunction, muscle weakness, reduced sensation and loss of bladder or bowel control.

In some embodiments, treatment of NMOSD includes the improvement of a clinical marker for NMOSD progression. These markers include, for example, time to relapse, annualized relapse rate (ARR), expanded disability scale score (EDSS), modified Rankin scale (mRS), quality of life (ED-5D), Hauser ambulatory index (HAI), change in visual acuity using a Snellen chart and severity of relapse using the optic spinal impairment score (OSIS).

NMOSD relapse is evidenced by symptoms of NMOSD occurring in a subject where symptoms have previously been successfully ameliorated. Relapse is shown by the onset or worsening of symptoms associated with vision or sensation. Changes in vision that are associated with relapse of NMOSD include rapid onset of eye pain, blurring of vision, colors that do not seem right, missing field of vision, spots or dots in the field of vision, flashing or flickering lights in the field of vision, difficulty focusing, difficulty reading and feelings that the field of vision seems incorrect. Changes in sensation that are associated with relapse of NMOSD include pain, tingling, numbness, arm, leg or face seems to fall asleep, loss of sense of position in space, loss of sense in extremities, slight touching is painful, clothes or bed sheets cause pain, and subject not being able to detect injury to the subject. Annualized relapse rate (ARR) is the average number of relapses per year.

In some embodiments, a subject treated for NMOSD has had three or more relapses in the 24 month period before eculizumab is administered. In some embodiments, a subject treated for NMO has 2, 3, 4, 5, 6, 7, 8, 9, 10, 11, 12 or more relapses in the 24 month period before eculizumab is administered. In some embodiments, a subject treated for NMOSD has an ARR of 1.0 or greater in the 24 month period before eculizumab is administered. In some embodiments, a subject treated for NMOSD has an ARR of at least 1.0, 1.2, 1.4, 1.6, 1.8, 2.0, 25, 3.0, 3.5, 4.0, 4.5, 5.0, 5.5, 6.0, 6.5. 7.0 or more in the 24 month period before eculizumab is administered.

Disability can be assessed based on the EDSS scores comparing the change from baseline in the two treatment groups. The Kurtzke Expanded Disability Status Scale (EDSS) is a method of quantifying disability in multiple sclerosis. The EDSS replaced the previous Disability Status Scales used in Multiple Sclerosis (MS). The EDSS quantified disability in eight Functional Systems (FS) and allows neurologists to assign a Functional System Score (FSS) in each of these.

The Functional Systems are: pyramidal, cerebellar, brainstem, sensory, bowel and bladder, visual, cerebral and other. EDSS steps 1.0 to 4.5 refer to people with MS who are fully ambulatory. EDSS steps 5.0 to 9.5 are defined by the impairment of ambulation. Disability is also to be assessed based on the mRS score comparing the change from baseline in the two treatment groups. mRS score is assessed by the treating physician at the protocol specified time points.

In some embodiments, a subject treated for NMOSD has an EDSS score of at least 1.0 in the 24 month period before eculizumab is administered. In some embodiments, a subject treated for NMOSD has an EDSS score of at least 1.0, 1.5, 2.0, 2.5, 3.0, 3.5, 4.0, 4.5, 5.0, 5.5, 6.0, 6.5. 7.0 or more in the 24 month period before eculizumab is administered. In some embodiments, a subject treated for NMOSD has an EDSS score from 1.0 to 7.0 in the 24 month period before eculizumab is administered. In some embodiments, a subject treated for NMOSD has an HAI score of at least 2.0 in the 24 month period before eculizumab is administered. In some embodiments, a subject treated for NMOSD has an HAI score of at least 0.0, 1.0, 2.0, 3.0, 4.0, 5.0, 6.0, 7.0, 8.0 or 9.0 in the 24 month period before eculizumab is administered. In some embodiments, a subject treated for NMOSD has an HAI score from 0.0 to 8.0 in the 24 month period before eculizumab is administered. In some embodiments, a subject treated for NMOSD has an mRS score of at least 0.0 in the 24 month period before eculizumab is administered. In some embodiments, a subject treated for NMOSD has an mRS score of at least 0.0, 1.0, 2.0, 3.0, 4.0, 5.0, or more in the 24 month period before eculizumab is administered. In some embodiments, a subject treated for NMOSD has an mRS score from 0.0 to 2.5 in the 24 month period before eculizumab is administered.

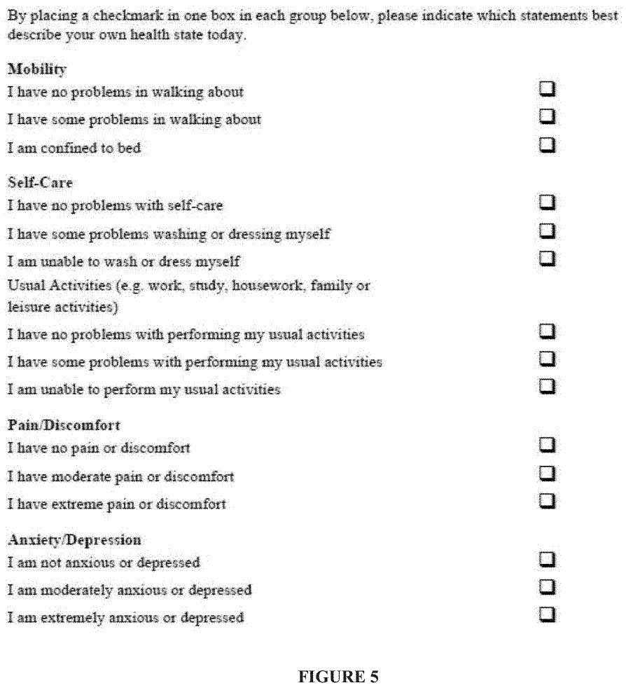

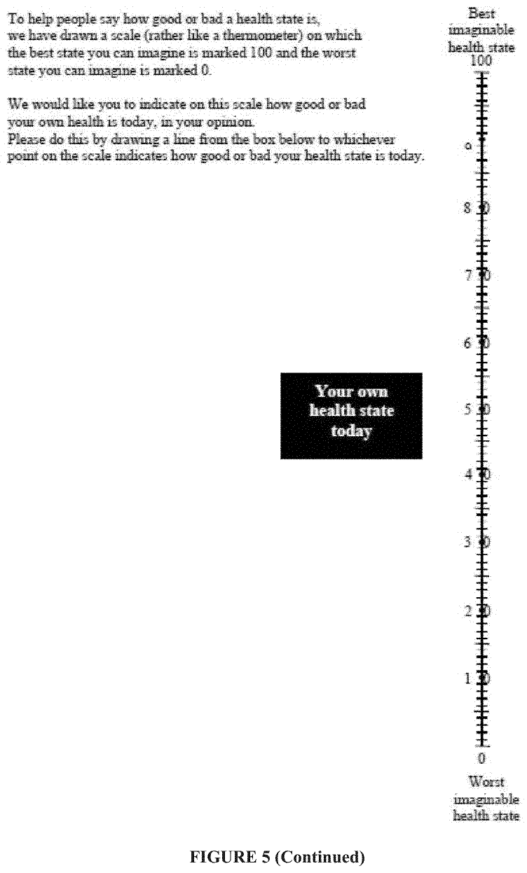

Quality of life (QOL) can be assessed by the patient self-assessment questionnaires EQ-5D and SF-36 at the protocol specified time points. A sample questionnaire for EQ-5D is shown in FIG. 5. The EUROQOL (EQ-5D) is a reliable and validated survey of health status in 5 areas: mobility, self-care, usual activities, pain/discomfort, and anxiety/depression, completed by the subject. Each area has 3 levels: level 1 (no problems), level 2 (some problems), and level 3 (extreme problems). The EQ-5D is administered at Day 1, Weeks 4, 8, 12, 24, 36, 48, 60, 72, 84, 96 and 104, or ET (Visits 2, 6, 8, 10, 16, 22, 28, 34, 40, 46, 52 and 56, or ET). A clinically meaningful improvement in a patient's EQ-5D would be reflected as an increase in score after 26 weeks of treatment. A sample questionnaire for SF-36 is shown in FIG. 5.

Ambulatory function can be assessed, for example, by HAI scale. Visual acuity can be assessed, for example, using the Snellen chart. Severity of relapse can be assessed, for example, using the optic spinal impairment score (OSIS). OSIS scores are summarized in Table 1.

According to some embodiments, subjects administered eculizumab show an increased time interval between relapses of NMOSD. In some embodiments, the subjects have period before relapse of greater than 6 weeks. In some embodiments, the period before relapse is greater than 6, 12, 18, 24, 30, 36, 42, 48, 54, 60, 66, 72, 78, 84, 90, 96, 102 or more weeks. In some embodiments, the period before relapse is greater than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or more weeks. In some embodiments, the period before relapse is greater than 1, 2, 3, 4, 5, 6, 7, 8, 9, 10, or more years. In some embodiments, the period before relapse is between 6 and 52 weeks, 6 and 26 weeks, 6 and 10 weeks, 26 and 52 weeks, 1 and 2 years, 1 and 5 years, 5 and 10 years or a relapse does not occur during the lifetime of the subject. In some embodiments, the period before relapse is greater than 6, 12, 18, 24, 30, 36, 42, 48, 54, 60, 66, 72, 78, 84, 90, 96, 102 or more months.

According to some embodiments, the course of treatment with eculizumab lasts for 108 weeks. According to other embodiments, the course of treatment lasts for 26-52, 26-78, 26-120, 26-130, 26-156, 26-104, 26-130, 26-156, 26-182, 26-208 weeks or more. In some embodiments, the course of treatment lasts for greater than 26, 27, 28, 29, 30, 31, 32, 33, 34, 35, 36, 37, 38, 39, 40, 41, 42, 43, 44, 45, 46, 47, 48, 49, 50, 51, 52, 78, 104, 130, 156 or 182 weeks. According to other embodiments, the course of treatment lasts for greater than 1, 2, 3, 4, 5, 10, 15, 20, 25, 30, 35, 40, 45, 50, 55, 60, 65, 70, 75, 80 or more years. In some embodiments, the course of treatment lasts for the remainder of the subject's life.

According to some embodiments, during the course of treatment, one or more symptoms or scores associated with NMOSD improves during the course of treatment and is maintained at the improved level throughout treatment. EDSS can improve, for example, after 26 weeks of treatment with a therapeutic antibody that specifically binds C5 and then remain at the improved level for the duration of the treatment, which can be, for example, 52 weeks of treatment with a therapeutic antibody that specifically binds C5. One example of a therapeutic antibody that binds C5 is eculizumab.

In some embodiments, the first sign of improvement occurs by 26 weeks of treatment with a therapeutic antibody that specifically binds C5. According to other embodiments, the first sign of improvement occurs between weeks 1-26, 26-52, 52-78, 78-104, 104-130, 130-156, 156-182, or 182-208 of treatment with a therapeutic antibody that specifically binds C5. In some embodiments, the first sign of improvement occurs at week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 78, 104, 130, 156 or 182.

According to some embodiments, the first sign of improvement is maintained for a number of weeks during treatment with a binding protein that specifically binds C5 such as eculizumab. According to some embodiments, this number of weeks is at least 26. According to other embodiments, this number of weeks is 1-26, 26-52, 52-78, 78-104, 104-130, 130-156, 156-182, or 182-208. In some embodiments, this number of weeks is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 78, 104, 130, 156 or 182. According to some embodiments, when the first sign of improvement is maintained, this means that the metric for treatment of NMOSD does not fall below the value of the first sign of improvement. The metric could continue to improve and this would still be defined as maintenance of the first sign of improvement.

According to some embodiments, the C5 binding protein can be administered to a subject suffering from NMOSD at between 600 and 6000 mg. According to other embodiments, the dose of eculizumab is between 900 and 1500 mg, 900 and 1300 mg, or 900 and 2000 mg. According to other embodiments, the dose of eculizumab is about 600, 700, 800, 900, 1000, 1100, 1200, 1300, 1400, 1500, 1600, 1700, 1800, 1900, 2000, 2500, 3000, 4000, 5000 or 6000 mg. According to other embodiments, the dose of eculizumab is at least 600, 700, 800, 900, 1000, 1100, 1200, 1300, 1400, 1500, 1600, 1700, 1800, 1900, 2000, 2500, 3000, 4000, 5000 or 6000 mg.

These doses can be administered once a month, once every two weeks, once a week, twice a week or daily. According to some embodiments, the dose is administered once every two weeks or once a week. According to other embodiments, eculizumab is administered to a subject suffering from NMOSD in a multiphase dosing regimen. According to some embodiments, the multiphase dosing regimen has 2, 3, 4, 6, 7, 8, 9, 10 or more phases. In some embodiments, each phase provides a higher dose than the phase before it.

In some embodiments, the eculizumab multiphase dosing regimen has two phases. The first phase is an induction phase. This phase provides a dose of 600 or 900 mg per week. In some embodiments, this phase lasts for 2, 3, 4, 5, 6, 7, 8, 9 or 10 weeks. In some embodiments, this phase lasts between 2 and 6 weeks. In some embodiments, the phase lasts for 5 weeks. According to some embodiments, the phase given any week is higher than the previous week. In some embodiments, the dose remains the same for a number of weeks and is then increased. In some embodiments the dose remains the same for the first 1, 2, 3, 4, 5, 6, 7, 8 or 9 weeks and is then increased. In some embodiments, the dose remains the same for the first 4 weeks. According to some embodiments, the eculizumab dose is between 600 and 1200 mg, 800 and 1500 mg, 900 and 1200 mg, 900 and 1100 mg, 900 and 1000 mg, 800 and 1000 mg, 800 and 1100 mg or 800 and 1200 mg for a number of weeks and is then increased. In one embodiment, the eculizumab dose is about 900 mg on day 1, followed 900 mg on day 7, 900 mg on day 14, 900 mg on day 21 and then increased to 1200 mg for the fifth dose on day 28 and then 1200 mg is administered every 14±2 days thereafter.

In one particular embodiment the eculizumab induction phase dosing regimen comprises five administered doses on the following schedule: 900 mg on day 1; 900 mg on day 7 (week 1); 900 mg on day 14 (week 2), 900 mg on day 21 (week 3) and 1200 mg on day 28 (week 4) and then 1200 mg is administered every 14±2 days thereafter. The actual days between each dose may vary during the induction by 1 or 2 days to accommodate unexpected events in the patients' schedule.

According to one embodiment, after the eculizumab dose is administered at about 900 mg once per week for four doses over 3 weeks it is then increased to 1200 per week for one week. According to another embodiment, after the eculizumab dose is administered at about 900 mg on day 1, and once per week for four doses over 3 weeks it is then increased to 1200 mg every two weeks.

According to this embodiment, the second phase of eculizumab dosing is the maintenance phase. The maintenance phase of eculizumab dosing can last for between 6 weeks and the life of the subject. According to other embodiments, the maintenance phase lasts for 26-52, 26-78, 26-104, 26-130, 26-156, 26-182, 26-208 weeks or more. In some embodiments, the maintenance phase lasts for greater than 26, 27, 28, 29, 30, 31, 32, 33, 34, 35, 36, 37, 38, 39, 40, 41, 42, 43, 44, 45, 46, 47, 48, 49, 50, 51, 52, 78, 104, 130, 156 or 182 weeks. According to other embodiments, the maintenance phase lasts for greater than 1, 2, 3, 4, 5, 10, 15, 20, 25, 30, 35, 40, 45, 50, 55, 60, 65, 70, 75, 80 or more years. In some embodiments, the maintenance phase lasts for the remainder of the subject's life.

In some embodiments, the eculizumab multiphase dosing regimen includes a third phase. This third phase is used when an NMOSD patient must undergo plasma exchange. In this phase after plasma is exchanged a dose of eculizumab is administered to replace the drug lost in plasma exchange. According to some embodiments, this eculizumab dose is between 300 and 1200 mg, 400 and 1500 mg, 500 and 1000 mg, 400 and 800 mg, or 500 and 700 mg. According to some embodiments, this eculizumab dose is about 600 mg. In some embodiments, the third phase, 600 mg eculizumab dose is administered within 1 hour after completion of plasmapheresis. In some embodiments, the third phase, 600 mg dose is administered within 2 hours after completion of plasmapheresis. In some embodiments, the third phase, 600 mg dose is administered within 3 hours after completion of plasmapheresis. In some embodiments, the third phase, 600 mg dose is administered within 4 hours after completion of plasmapheresis. In some embodiments, the third phase, 600 mg dose is administered within 5 hours after completion of plasmapheresis. In some embodiments, the third phase, 600 mg dose is administered within 6 hours after completion of plasmapheresis.

In another aspect, a patient switches from receiving one C5 inhibitor to a different C5 inhibitor during the course of treatment. Different anti-C5 antibodies may be administered during separate treatment periods. For example, in one embodiment, a use of eculizumab for treating a human patient having a complement-associated disorder (e.g., generalized myasthenia gravis (gMG)) who is being treated with eculizumab is provided, the method comprising discontinuing treatment with eculizumab and switching the patient to treatment with an alternative complement inhibitor. For example, in one embodiment, the patient is treated with eculizumab during a treatment period (e.g., for 26 weeks), followed by treatment with another anti-C5 antibody during an extension period. In one embodiment, eculizumab is administered to the patient at a dose of 900 mg on Days 1, 8, 15, and 22 of the administration cycle during an induction phase, followed by a maintenance dose of 1200 mg of eculizumab on Day 19 of the administration cycle and every two weeks thereafter (e.g., for a total of 26 weeks), followed by treatment with another anti-C5 antibody for an extension period of up to two years.

### Pharmaceutical Compositions

Pharmaceutical compositions comprising a C5 antibody, e.g., eculizumab, either alone or in combination with prophylactic agents, therapeutic agents, and/or pharmaceutically acceptable carriers are provided. The pharmaceutical compositions comprising eculizumab provided herein are for use in, but not limited to, diagnosing, detecting or monitoring a disorder; in preventing, treating, managing or ameliorating a disorder or one or more symptoms thereof; and/or in research. The formulation of pharmaceutical compositions, either alone or in combination with prophylactic agents, therapeutic agents, and/or pharmaceutically acceptable carriers, is known to one skilled in the art.

Methods of administering a prophylactic or therapeutic agent provided herein include, but are not limited to, parenteral administration (e.g., intradermal, intramuscular, intraperitoneal, intravenous and subcutaneous), epidural administration, intratumoral administration, mucosal administration (e.g., intranasal and oral routes) and pulmonary administration (e.g., aerosolized compounds administered with an inhaler or nebulizer). The formulation of pharmaceutical compositions for specific routes of administration, and the materials and techniques necessary for the various methods of administration are available and known to one skilled in the art.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. The term “dosage unit form” refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms provided herein is dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of eculizumab provided herein is 600-5000 mg, for example, 900-2000 mg. It is to be noted that dosage values may vary with the type and severity of the condition to be alleviated. It is to be further understood that for any particular subject, specific dosage regimens may be adjusted over time according to the individual need and the professional judgment of the person administering or supervising the administration of the compositions, and that dosage ranges set forth herein are exemplary only and are not intended to limit the scope or practice of the claimed composition.

### Combination Therapy

A C5 binding protein provided herein can be administered with one or more additional medicaments or therapeutic agents useful in the treatment of NMOSD, the additional agent being selected by the skilled artisan for its intended purpose. For example, the additional agent can be a therapeutic agent art-recognized as being useful to treat the disease or condition being treated by the antibody provided herein. The combination can also include more than one additional agent, e.g., two or three additional agents.

In some embodiments, a C5 antibody, e.g., eculizumab is administered with an immunosuppressive therapy (IST). ISTs include steroids, azathioprine (AZA), and mycophenolate mofetil (MMF). Steroids include corticosteroids like prednisone, methylprednisone IV or pulse-high dose steroids administered intravenously. Each of these ISTs are any combination are administered at therapeutically effective doses. In some embodiments, the C5 antibody, e.g., eculizumab is administered with a steroid and AZA and/or MMF.

### EXAMPLES

### Example 1. Effectiveness of Eculizumab in Treating Neuromyelitis Optica in Human Subjects

The PREVENT study (ClinicalTrials.gov, number NCT01892345; EudraCT, number 2013-001150-10) was a phase 3, randomized, double-blind, parallel-group, placebo-controlled, time-to-event trial (NCT01892345) conducted at 70 sites, primarily hospital clinics, in 18 countries worldwide. The study was conducted between Apr. 11, 2014, and Jul. 17, 2018 to evaluate the safety and efficacy of eculizumab in patients with relapsing NMO. Eligible patients were randomized 2:1 to one of two parallel treatment arms: 1) eculizumab infusion or 2) placebo infusion. Patients were allowed to receive stable maintenance dose of protocol permitted supportive ISTs for relapse prevention, as defined by their Treating Physician. Patients were required to remain on that dose for the duration of the study or until the patient experienced a relapse.

Based on power calculations for this study, the study was designed to continue until 24 relapses (in 24 distinct subjects) occurred, as adjudicated by a committee external to the Sponsor and blinded to treatment. Therefore, the enrollment was closed when either 24 adjudicated events occurred or when up to 132 patients were enrolled, whichever occurred first. The trial duration was estimated to take approximately 3-4 years including enrollment. In this time-to-event trial, the trial duration for an individual patient varied depending on when the patient entered the trial and on the patient's outcome. The course of the trial for an individual patient consisted of: Screening Period, Study Period, and Safety Follow-up Period (for patients who withdrew from this trial or for patients who did not enter the extension trial). The End of Study (EOS) Visit for an individual patient took place when one of the following conditions were met, whichever occurred first: (a) the patient experienced an On-Trial Relapse as defined by Section 5.1.1; or (b) the trial ended by meeting 24 Adjudicated On-Trial Relapse events (in 24 distinct patients). Some patients were provided with the opportunity to participate in an extension trial (separate protocol ECU-NMO-302) to receive eculizumab after completion of the EOS Visit; patients who terminated the study early were not eligible for the extension trial.

PREVENT was conducted in accordance with the Declaration of Helsinki, the International Conference on Harmonisation guidelines for Good Clinical Practice, and applicable regulatory requirements. Study documents were approved by all institutional review boards. Patients provided written informed consent before participation. The Sponsor designed the study in consultation with two authors. An independent data monitoring committee assessed risks and benefits for patients by evaluating cumulative unblinded safety data at regular intervals and on an ad hoc basis. Data were collected by investigators and other site staff, and analyzed by the Sponsor.

### 1. Investigational Plan

### 1.1 Overall Trial Design and Plan

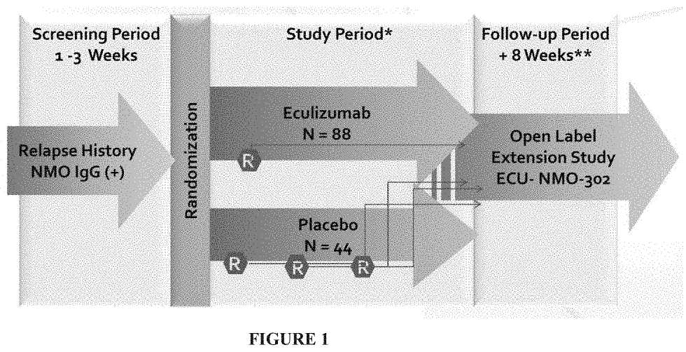

Described herein was a randomized, double-blind, parallel-group, placebo-controlled, multicenter, time-to-event trial to evaluate the safety and efficacy of eculizumab in patients with relapsing NMO. There were three periods in the trial including: Screening Period, Study Period and Follow-up Period (only for patients who withdraw from this trial or patients who do not enter the extension trial). These periods were summarized in FIG. 1. Patients who completed the Study Period had the opportunity to enter an extension trial (separate protocol) to receive open-label eculizumab. Patients entering the extension trial underwent a blinded induction phase similar to the induction in this trial to preserve the blinded nature of this trial. The protocol was finalized on Dec. 31, 2012. Significant amendments to the protocol are detailed below.

Mar. 15, 2013 (Amendments for all Countries; Before Patient Enrollment)

Trial design: updated the strata for EDSS from three strata (≤2.0 vs. ≥2.5 to ≤5.5 vs. ≥6.0) to two strata (≤2.0 vs. ≥2.5 to ≤7.0); updated the strata for IST status from two strata (no IST or receiving only steroids vs. IST with/without steroids) to three strata (patients who are treatment naïve vs. patients who have failed ISTs and are receiving IST at randomization vs. patients who have failed ISTs and are not receiving IST at randomization); updated definition of treatment naïve; increased the duration of the screening period from 1-2 weeks to 1-3 weeks; included the option to perform an interim analysis when a minimum of 70% of expected events had occurred in order to stop the trial for efficacy; provided clarification on the visit. interval between the PREVENT study and the extension study.

Trial procedures: allowed the use of methotrexate and tacrolimus as supportive therapy; provided a patient education card that detailed the signs and symptoms of a potential relapse, with instructions to contact the investigator at the first sign or symptom of a potential relapse; deleted the restriction that relapses occurring within the first 14 days of study drug administration would not count as events; added pharmacokinetic (PK)/pharmacodynamic (PD) sampling at relapse evaluation visits.

End points: added change from baseline to end of study in Modified Rankin Scale (MRS) score as a secondary end point; deleted the secondary end point of change from baseline to end of the study in visual function as measured by visual acuity; added the Columbia-Suicide Severity Rating Scale as a safety outcome measure.

Statistical analyses: incorporated new sensitivity analyses for the primary efficacy end point.

May 15, 2013 (Amendments for all Countries; Before Patient Enrollment)

Patients: deleted inclusion criterion #6 around ISTs

Trial design: updated the randomization strata relating to IST status from patients who are treatment naïve vs. patients who have failed ISTs and who are receiving IST at randomization vs. patients who have failed ISTs and who are not receiving IST at randomization to patients who are treatment naïve vs. patients receiving prior IST and who are receiving IST at randomization vs. patients receiving prior IST and who are not receiving IST at randomization.

Trial procedures: updated to indicate that the data monitoring committee would receive reports concerning patients who have dropped out and any patients who have missing primary and secondary efficacy data; updated to indicate that the data monitoring committee would also review summaries of all patient-reported relapses to ensure no bias occurred in relapse assessments by treating physicians.

Jun. 21, 2013 (Amendments for Study Centers in Germany; Before Patient Enrollment)

Trial procedures: updated to clarify that the chest X-ray at screening was to be performed only if clinically indicated at the discretion of the investigator.

Jul. 10, 2013 (Amendments for Study Centers in Japan; Before Patient Enrollment)

Trial design: additional detail provided to indicate that patients were to be informed of an increased risk of certain types of infections during treatment with eculizumab.

Trial procedures: guidance was added for patients to report any potential sign or symptom of illness, and that patients reporting symptoms of a possible infection should be evaluated immediately; text revised to address comment from the Pharmaceuticals and Medical Devices Agency that the protocol should apply more precise criteria for infection.

Oct. 16, 2013 (Amendments for all Countries)

Patients: updated the inclusion criterion around historical relapses to be less restrictive; updated the inclusion criterion around concomitant IST use during the study to be less restrictive and better aligned with current clinical practice; added exclusion criterion regarding previous steroid use, as high corticosteroid doses may decrease the relapse rate in some patients, confounding the interpretation of the study results.

Trial design: increased the number of study centers from 100 to 100-120 to improve recruitment; updated the randomization strata relating to IST status from patients who are treatment naïve vs. patients receiving prior IST and who are receiving IST at randomization vs. patients receiving prior IST and who are not receiving IST at randomization to patients who are treatment naïve vs. patients continuing on the same IST since last relapse vs. patients with changes in IST since last relapse.

Trial procedures: expanded the clinical information to be collected for historical relapses to aid in describing the patients, their disease severity, prior treatments, and more accurate subgroup assignment based on their treatment history; extended safety follow-up from 4 to 8 weeks based on the PK characteristics of eculizumab.

Statistical analysis: updated assumptions for sample size and event-driven power calculations following further consideration of the previous independent study with eculizumab and incorporating new and unpublished information from two academic databases; updated the anticipated sample size based on power calculations requiring 24 relapses in 24 different patients.

Dec. 16, 2014 (Amendments for Study Centers in the Czech Republic)

Trial procedures: removed sample collection for PK/PD/free complement protein C5 analysis and clarified that no PK/PD/free C5 samples will be collected in the Czech Republic; removed optional cerebrospinal fluid sample collection and analysis and clarified that no cerebrospinal fluid samples will be collected in the Czech Republic.

Feb. 25, 2015 (Amendments for all Countries)

Patients: changed an inclusion criterion from NMO-immunoglobulin G (IgG)-seropositive at screening visit to NMO-IgG seropositive to allow the inclusion of patients who were historically seropositive. [Communications to the study centers indicated: “Only patients with a positive test for NMO-IgG (aquaporin-4 [AQP4] antibody) obtained prior to or during screening will be included in the study. Only validated diagnostic tests performed by a qualified laboratory are acceptable.” Supporting documentation regarding the test results, the laboratories, and the assays were required in order to ensure that the NMO-IgG (AQP4-IgG) serostatus of patients was established in accordance with accepted scientific and global regulatory standards.]

Trial design: expanded the number of study centers from 100-120 to 120-150 to improve recruitment; increased the duration of the screening period from 1-3 weeks to 1-6 weeks to allow more time for procedures to be completed and for patients to be vaccinated at least 2 weeks before the administration of study treatment, as required by the protocol.

Trial procedures: allowed qualified non-physician healthcare professionals (e.g. nurses) to complete the EDSS assessment with the Sponsor's approval; removed text that emphasized reporting IST use within the 24 months before screening to encourage investigators to obtain all available history on IST use for relapse prevention; clarified that supportive IST use was for relapse prevention, so as not to be confused with IST administered for relapse treatment or other medical reasons; provided some flexibility for the supplemental study drug dose administration time from within 60 minutes to preferably within 1-2 hours after each cycle of plasma exchange to address operational challenges.

Statistical analysis: removed interim analysis following recommendations from regulatory agencies; changed the definition of the per-protocol set from patients who have no major protocol deviations or inclusion/exclusion criteria deviations to no major protocol deviations or key inclusion/exclusion criteria deviations that might potentially affect efficacy to prevent the exclusion of patients from the per-protocol set with deviations not relevant for assessing the efficacy and safety of eculizumab.

Jan. 4, 2016 (Amendments for Thailand)

Patients: expanded the exclusion criterion regarding meningococcal infections to exclude patients with a history of resolved meningococcal infections in response to requirements set forth by the Thailand Ethics Committee.

Jul. 1, 2016 (Amendments for all Countries)

Trial procedures: established the relapse adjudication committee (RAC) for adjudication of all on-trial relapses.

End points: updated the primary end point of the study from time to first relapse to time to first adjudicated on-trial relapse; changed the secondary efficacy end point from annualized relapse rate (ARR) to adjudicated ARR.

Statistical analysis: added a sensitivity analysis for the primary end point (time to first on-trial relapse, analyzed using a log-rank test and including strata for the randomization stratification variables); added a sensitivity analysis for ARR using all on-trial relapses in a Poisson regression analysis with treatment group, stratification variables, and baseline ARR as covariates in the model, and the log of time in the study as the offset variable.

### 1.2. Screening Period

At the screening visit, after obtaining the informed consent from the patient, the patient was screened for trial eligibility through medical history review, demographic data, ECG and laboratory assessments. The medical history review included confirmation of the diagnosis of NMO or NMO Spectrum Disorder (NMOSD) as defined by 2006 or 2007 criteria (Wingerchuk, D. et al., Neurology, 66:1485-9, 2006; Wingerchuk, D. et al., Neurology, 66:603-5, 2007). Overall, 213 patients were screened, 143 randomized (90.9% female, median age 45.0 years) and 124 completed the study (eculizumab 80/96 [83.3%], placebo 44/47 [93.6%]).

### 2006 Diagnostic Criteria for NMO

Definite NMO
Optic neuritis
Acute myelitis
At least two of three supportive criteria:
1. Contiguous spinal cord MRI lesion extending over 3 vertebral segments
2. Brain MRI not meeting diagnostic criteria for multiple sclerosis
3. NMO-IgG seropositive status

### 2007 Diagnostic Criteria for NMO Spectrum Disorder NMO

Limited forms of NMO:
Idiopathic single or recurrent events of longitudinally extensive myelitis (≥3 vertebral segment spinal cord lesion seen on MRI)
Optic neuritis: recurrent or simultaneous bilateral
Asian optic-spinal multiple sclerosis
Optic neuritis or longitudinally extensive myelitis associated with systemic autoimmune disease
Optic neuritis or myelitis associated with brain lesions typical of NMO (hypothalamic, corpus callosal, periventricular, or brainstem)

Detailed information on relapses within the two years prior to screening were assessed by the Investigator to determine if they met the criteria for Historical Relapse Definition as specified by this protocol (see Section 1.5.1.). Detailed information related to relapses within the prior two years was collected, if available. This included date of onset and its clinical presentation for each relapse (e.g., optic neuritis (ON), transverse myelitis (TM), longitudinally extensive transverse myelitis (LETM), or brainstem event); and EDSS at the following time points: prior to relapse, at nadir and during recovery, for severity of relapse and recovery. Start/stop dates and dose regimen of all IST(s) including immunomodulatory agents and non-drug therapies taken for relapse prevention or treatment of a relapse within the prior two years were also collected and recorded. If plasmapheresis (PE) was administered for treatment of a relapse, the number of cycles of PE was also collected. Information on all other previous historical relapses including relapse onset date and its clinical presentations, name/type of IST(s) at the time of relapse and treatment received for the acute relapse was also collected, if available. Screening laboratory assessments included the verification of seropositive for NMO-IgG.

Investigators identified relapses or cases of interest (potential relapses) were referred to an independent Relapse Adjudication Committee (RAC). The independent RAC then rendered a final decision. Data were analyzed in terms of Investigator diagnosed relapse as well as RAC determined relapse. Discordance between treating physicians and the committee in identifying relapses was anticipated owing to the challenges in diagnosing NMOSD relapses and because physicians were inclined to take a conservative approach to avoid the severe consequences of untreated relapses. Implementation of the adjudication committee was thus an important element of study design. Credibility of the primary end point findings was enhanced by the fact that treatment effects in the overall population were significant whether relapses were identified by physicians or blinded adjudication committee.

Supportive ISTs were allowed during the trial under certain restrictions. The following ISTs were allowed either as mono-therapy or in combination such as corticosteroids: azathioprine (AZA), mycophenolate mofetil (MMF), methotrexate, tacrolimus, cyclosporine and cyclophosphamide. If a patient entered the trial receiving IST(s), the patient was on a stable maintenance dose of IST(s), as defined by their Treating Physician, prior to the Screening Visit. No new IST(s) were permitted during the trial unless a patient experienced a relapse. IST and/or therapies for NMO relapses (either acute treatment or prevention) within two years prior to screening and all other medications taken within 30 days of screening were reviewed and recorded on the CRF. Non-drug therapy for NMO relapse prevention or treatment within 24 months prior to screening were also collected and recorded on CRF. If PE was administered for treatment of a relapse, the number of cycles of PE was also collected and recorded on the CRF.

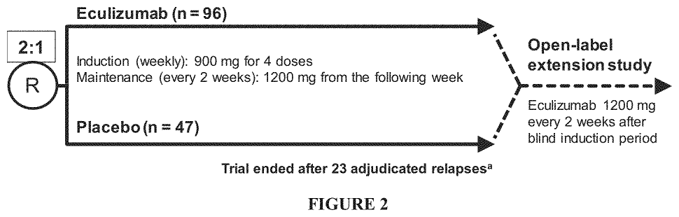

If all inclusion criteria and none of the exclusion criteria were met, patients were vaccinated against N. meningitidis (if not already vaccinated within the time period of active coverage specified by the vaccine manufacturer). Patients were vaccinated at least 14 days prior to receiving the first dose of study medication or vaccinated and received treatment with appropriate antibiotics until 14 days after the vaccination. The vaccination schedule is summarized in FIG. 2.

Patients who experienced a relapse during the screening period were considered a screening failure. Such patients might be rescreened for enrollment into the trial after receiving treatment for the relapse and when, in the opinion of the Investigator, the patient is medically stable. The patient must meet the enrollment criteria at rescreening to enter the trial.

### 1.3. Study Period

All patients who were vaccinated and cleared for randomization by their Principal Investigator (PI) and by the Sponsor's Medical Monitor were randomized on Day 1 on a 2:1 basis to the Eculizumab Arm or the Placebo Arm. A randomization worksheet was provided and the Sponsor's approval was required to ensure proper randomization. The randomization was across centers. The randomization stratification included two variables: 1) EDSS score at randomization (Day 1); and 2) patients' prior IST and IST status at the randomization (Day 1).
1. Patients' EDSS score at randomization (Day 1)
a. EDSS scores ≤2.0
b. EDSS scores ≥2.5-≤7.0
2. Patients' prior IST and IST status at randomization (Day 1)
a. Treatment naïve patients (i.e., patients with no prior or current ISTs, except steroids alone)
b. Patients continuing on the same IST(s) since the last relapse (i.e., including patients with dose adjustments since the last relapse)
c. Patients with changes in IST(s) since last relapse (i.e., switched IST(s) [e.g., AZA to MMF], added IST [e.g., corticosteroid], or withdrew any IST treatment)

Patients received either eculizumab or placebo according to the randomization assignment and the regimen described below. The treatment duration for an individual patient varied in this time-to-event trial. All patients remained on randomized treatment assignment until the EOS Visit.

Identification of potential relapse was critical for patient safety and for the trial. Patients received a Patient Education Card that detailed signs and symptoms of a potential relapse and instructions to contact the Investigator or his/her designee at the first sign or symptom of a potential relapse. The Investigator or his/her designee reviewed this information and any additional warning signs of a relapse specific to that patient's clinical picture at each visit. Patients were ideally evaluated within 24 hours of notification of the Investigator of a possible relapse, and no later than 48 hours. The Treating Physician was responsible for making the decision as to whether the clinical signs, symptoms and neurologic change (objective findings on neurologic examination) met the definition for On-Trial Relapse and treated the patient's relapse according to the recommended Standardized On-Trial Relapse Treatment regimen. Follow-up visits to monitor the course of the relapse were performed at 1, 4 and 6 weeks after the onset of relapse. Additional (Unscheduled) Follow-up Relapse Evaluation Visits were permitted and were made at the discretion of the Treating Physician. All reports of possible relapses and actions taken for the possible relapse were documented in the patient's medical chart or source documents and recorded in the CRF.

As this is a time-to-event trial, patients who experienced a relapse had met the primary endpoint and were discontinued from this trial after completion of the Week 6 Relapse Evaluation Visit. Thus, the Week 6 Relapse Evaluation Visit also served as the EOS Visit for these patients.

For patients who did not have relapses, the EOS Visit was completed when the trial ended by meeting the maximum number of relapses expected (24 relapses in 24 distinct patients). Patients who completed the trial were provided with an opportunity to enter an extension trial (separate protocol) to receive open-label eculizumab. The visit interval between this trial and the extension trial is two weeks from the last IP administration, without interruptions in IP dosing. Thus, patients who exit the trial due to a relapse had their first extension visit once the Week 6 Relapse Evaluation Visit is completed and no later than 2 weeks (14 days±2 days) after the last IP dose; patients who exit this trial due to trial completion have their first extension visit 2 weeks (14 days±2 days) after the EOS Visit. All patients entering the extension trial had to undergo a blinded eculizumab induction period similar to the induction in this trial to preserve the blind treatment assignment of this trial.

### 1.3.1. Relapse Evaluation by Examining and Treating Physicians

Patients were instructed to contact the Investigator or his/her designee at the first sign or symptom of a potential relapse. Patients ideally were evaluated within 24 hours of notification of the investigator of a possible relapse, and no later than 48 hours. All potential relapses were evaluated by both the Examining and the Treating Physicians. All reports of possible relapses and actions taken for the possible relapse were documented in the patient's medical chart and recorded in the CRF.

At each Relapse Evaluation Visit, the blinded Examining Physician performed a complete neurologic exam and documented the FSS and the EDSS score. The Treating Physician performed a complete neurologic examination and documented the Optic-Spinal Impairment Score (OSIS). The Treating Physician or appropriately trained designee performed the visual acuity test (Snellen chart) and HAI.

The Treating Physician determined if the clinical signs, symptoms and neurologic change (objective findings on examination) met the definition for On-Trial Relapse as outlined in this protocol. The recommended treatment regimen for confirmed relapse outlined in this protocol was initiated after all specified relapse evaluation procedures were completed. Follow-up Relapse Evaluation Visits were performed at 1, 4 and 6 weeks after the onset of relapse. To monitor the course of relapse recovery, additional Relapse Evaluation Visits were permitted at the discretion of the Treating Physician and were documented in the patient's medical chart and recorded in the CRF.

All suspected events requiring adjudication were identified by the Treating Physician as an On-Trial Relapse. Event packages for adjudication were assigned a unique identification number and included the information listed below. RAC members first independently reviewed and adjudicated each event package. In order to ensure consistency and objectivity in the adjudication process, RAC members did not discuss events among themselves during Phase 1 review.
Prior and concomitant medications
Medical history including prior relapse events.
Additional data listings from the eCRFs including but not limited to: Adverse Events (AEs), Treating Physician's Neurological exam, EDSS rater (or Examining Physician)'s FS scoring, EDSS scores, HAI, mRS, OSIS scores.
SAE report of the relapse(s), if applicable.
Copies of correspondence with the sites relevant to the adjudication of the event.
Copies of reports and imagery (where available) e.g. MRI, OCT and VF reports.

If needed, additional data were requested.

Phase 1

Each complete event package was assigned to all three RAC Adjudicators. RAC Adjudicators would login and access events packages posted to the SharePoint site.

Each RAC Adjudicator would independently review the complete event package, render an adjudication decision on the Phase 1—NMO Relapse Adjudication Form, provide comments in sections A-C of the form, and sign/date the form. Complete and signed forms were to be scanned and submitted via email to the CEVA RAC Lead. This form would capture information on relevant clinical findings and would document the adjudicators' assessment of whether or not specific protocol criteria were met. The relapse would be assessed according to the RAC member's impression of the clinical data, as guided by the protocol definition of relapse.

If the adjudicator could not render a decision based on the event package provided, the adjudicator should indicate that additional information was required and list the information that was needed on the Phase 1—NMO Relapse Adjudication Form. Once this request was received by the CEVA RAC Lead, the CEVA RAC Lead would notify all RAC adjudicators that a member had requested additional information, and their review for this event should be put on hold until the additional information was provided. Where additional information was available, it was provided to all adjudicators. If all reasonable measures to collect additional documentation regarding an event failed, the RAC Adjudicators would review and render an adjudication decision based on best available data. More than one request could be made for each relapse, either by the same or by a different adjudicator.

If additional information was requested by one of the adjudicators, then that adjudicator would complete an additional Phase 1—NMO Relapse Adjudication Form to indicate their decision following receipt of the additional information or determination that the requested information was not available (i.e., they would indicate their ‘yes’ or ‘no’ decision regarding their adjudication of the relapse). In the event an adjudicator has completed a Phase 1 Form when additional information was being requested, he/she would complete a new Phase 1 Form upon receipt of the additional information.

If, after rendering a Phase 1 decision, any of the adjudicators wished to further discuss an event, the adjudicator should indicate that committee discussion was required on the Phase 1 form.

If the adjudication result determined by each independent RAC Adjudicator was in agreement during Phase 1, adjudication of that event was considered to be complete and a final adjudication outcome was rendered.

Phase 2

If the adjudication result determined by each independent RAC adjudicator was discordant and/or if an adjudicator indicated on the Phase 1 form that further discussion was required, the RAC adjudicators would discuss the event through a formal RAC teleconference meeting until they reached consensus or agreed that they were unable to reach final consensus. In the event that consensus could not be reached through discussion, the decision of the majority would be recorded as the final decision. The CEVA RAC Lead would populate the Phase 2—NMO Relapse Adjudication Form (Appendix 2) with the responses provided by the RAC. The RAC Chair would be responsible for approving and signing the form. Once the Phase 2 form had been approved and signed by the Chair, the CEVA RAC Lead will populate the Final Outcome Form.

The CEVA RAC Lead and the RAC members should comply with the following guidelines for the formal adjudication meetings:
The CEVA RAC Lead would distribute an agenda prior to the meeting listing all events to be reviewed for which agreement was not obtained via the independent review process. The agenda would also contain any requests for information from the individual members such that all members would have the same information before the meeting. The members should review this information prior to the adjudication meeting.
During the meeting, the CEVA RAC Lead's role would be limited to facilitating the discussion of each event; the CEVA RAC Lead was not to influence the decisions during the discussions.
The CEVA RAC Lead should request each member to briefly describe the rationale for the decision submitted during the independent review so that the committee could come to a decision on each event.
The CEVA RAC Lead would populate the Phase 2—NMO Relapse Adjudication Form (Appendix 2) on behalf of the RAC. The RAC Chair would be responsible for approving and signing the form.
If necessary, the RAC members might generate a request for more information before or during the meeting. In the event that this occurs, it would be captured in the meeting minutes and the event would be set aside for a future meeting date after the request had been fulfilled. This might occur at the next scheduled teleconference or an ad hoc meeting may be scheduled.
Following each meeting, the CEVA RAC Lead would distribute minutes to the RAC indicating a brief summary of the discussion.
The CEVA RAC Lead should escalate to the Sponsor if these discussions were not believed to be objective or not proceeding as intended.

Once an event was discussed at a Phase 2 teleconference, if follow-up discussions of that particular event were needed they may occur by email at the discretion of the RAC Chair. If this occurred, all RAC members and the CEVA RAC Lead must be copied on all correspondence related to the discussion of the event.

Final Outcome

All final decisions were captured on the RAC NMO Relapse Outcome Form, which was utilized for analysis and reporting. Decisions from the signed Phase 1 and Phase 2 forms were carried over onto the RAC NMO Relapse Outcome Form and it was considered complete.

### 1.4. Follow-Up Period (Post-Treatment)

If patients withdrew from this trial after receiving any amount of IP or did not enter the extension trial after completing this trial, a follow up visit for safety assessments was required at eight weeks after the last dose of IP. If a patient was discontinued due to an AE, the event was followed until it was resolved or, in the opinion of the PI, was determined to be medically stable.

### 1.5. Standard Protocol Definitions

Abbreviations and definitions for the study and follow-up period are provided in Table 35.

1.5.1. Historical Relapse Historical relapses occurred prior to the Screening Visit. For this protocol, historical relapse was defined as new onset of neurologic symptoms or worsening of existing neurologic symptoms with an objective change on neurologic examination (clinical findings or MRI findings or both) that persisted for more than 24 hours and/or the new onset of neurologic symptoms or worsening of existing neurologic symptoms that required treatment. Treatment was defined as use of high-dose IV steroids, PE, or IVIg.

### 1.5.2. On-Trial Relapse

On-Trial Relapses were acute attacks that occurred during the trial. For this protocol, On-Trial Relapse was defined as new onset of neurologic symptoms or worsening of existing neurologic symptoms with an objective change (clinical sign) on neurologic examination that persisted for more than 24 hours as confirmed by the Treating Physician. The signs and symptoms had to have been attributed to NMOSD, i.e., not caused by an identifiable cause such as infection, excessive exercise, or excessively high ambient temperature. The relapse was preceded by at least 30 days of clinical stability.

### 1.5.3. Severity of Relapse

Severity and degree of recovery of an On-Trial Relapse were measured by the OSIS. OSIS scores are summarized in Table 1.

TABLE 1
Severity and Degree of Recovery Measured by OSIS
Attacks during study are classified as minor or major (for purpose
of analysis) based upon their clinical severity as measured by the
Optico-Spinal Impairment Scale score (OSIS)

| | | | Attack |
|---|---|---|---|
| | Pre-attack | Post-attack | Descriptor |
| a) Severity | | | |
| Optic Neuritis | | | |
| V_{A} Subscale | | | |
| Score | | | |
| | 0-1 | 0-2 | Minor |
| | 0-1 | 3+ | Major |
| | 2-7 | Increase by 1 point | Minor |
| | 2-7 | Increase by 2+ points | Major |
| Myelitis | | | |
| Motor | | | |
| Subscale Score | | | |
| | 0-1 | 0-2 | Minor |
| | 0-1 | 3+ | Major |
| | 2-6 | Increase by 1 point | Minor |
| | 2-6 | Increase by 2+ points | Major |
| Sensory Score | | | |

Based on proprioceptive loss only: major attack if severe loss in one or
more limbs(s) with prior normal function or mild proprioceptive loss.

| | Attack | | Recovery |
|---|---|---|---|
| | (at Worst) | Improvement | Descriptor |
| b) Degree of | | | |
| Recovery | | | |
| Optic Neuritis | | | |
| V_{A} Subscale | | | |
| Score | | | |
| | 0-2 | 1-2 | Minor |
| | 3+ | >2 | Major |
| | 3+ | 1-2 | Minor |
| Myelitis | | | |
| Motor | | | |
| Subscale Score | | | |
| | 0-2 | 1-2 | Minor |
| | 3+ | >2 | Major |
| | 3+ | 1-2 | Minor |
| Sensory Score | | | |

Based on proprioceptive loss only: major improvement is considered
improvement >2 and minor improvement 1-2 compared with nadir.

The OSIS Visual Acuity (VA) Subscale Scores used to categorize the severity of Optic Neuritis (ON) are summarized in Table 2. The OSIS Motor Subscale Scores and Sensory Subscale Scores were used to categorize the severity of Transverse Myelitis (TM). Severity was assessed at the time of the relapse.

TABLE 2
OSIS Visual Acuity (VA) Subscale Scores
Visual Acuity (VA)

| | |
|---|---|
| 0 | Normal |
| 1 | Scotoma but VA (corrected) better than 20/30 |
| 2 | VA 20/30-20/59 |
| 3 | VA 20/60-20/100 |
| 4 | VA 20/I0I-201200 |
| 5 | VA 20/20I-20/800 |
| 6 | Count fingers only |
| 7 | Light perception only |
| 8 | No light perception |

Motor Function

| | |
|---|---|
| 0 | Normal |
| 1 | Abnormal signs (hyperreflexia, Babinski sign) without weakness |
| 2 | Mild weakness (MRC grade 5− or 4+) in affected limbs(s) |
| 3 | Moderate weakness (grade 3 or 4) in 1 or 2 UMN muscles in affected limbs(s) |
| 4 | Moderate weakness (grade 3 or 4) in 3 UMN muscles in affected limbs(s) |
| 5 | Severe weakness (grade 2) in 1 or more muscles in affected limbs(s) |
| 6 | Some plcgic (grade 0 or 1) muscles in 1 or more limbs |
| 7 | Plegia (grade 0 or 1) of all muscles in 1 or more limbs |

Sensory Function

| | |
|---|---|
| 0 | Normal |
| 1 | Mild decrease in vibration |
| 2 | Mild decrease in pinprick/temperature/proprioception or moderate decrease in vibration |
| 3 | Moderate decrease in touch/pin/proprioception or essentially lost vibration sense |
| 4 | Loss of all sensory modalities |
| 5 | Unknown |

Sphincter Function

| | |
|---|---|
| 0 | Normal |
| 1 | Mild urinary urgency or hesitancy; constipation |
| 2 | Moderate urinary urgency, hesitancy, or retention of bladder or bowel, infrequent urinary |
| | incontinence (less than once/week) |
| 3 | Frequent incontinence or retention requiring intermittent bladder catheterization or |
| | agressive (manual) bowel assistance |
| 4 | Indwelling urinary catheter or absence of sphincter control |
| 5 | Unknown |

### 1.5.4. Patient Population/Immunosuppressant Therapy Status

There were three patient populations based on prior IST and IST status at the time of randomization.
a) Treatment naïve patients (i.e., patients with no prior or current ISTs, except steroids alone)
b) Patients continuing on the same IST(s) since the last relapse (i.e., including patients with dose adjustments since the last relapse)
c) Patients with changes in IST(s) since the last relapse (i.e., switched IST(s) [e.g., AZA to MMF], added IST [e.g., corticosteroid], or withdrew any IST treatment)

### 1.5.4.1. Treatment Naïve Patients

Treatment naïve patients were defined as those who received or were receiving either no ISTs or had received only corticosteroids following treatment of acute relapses prior to the screening.

### 1.5.4.2. Patients Continuing on the Same IST at the Time of Randomization

Patients continuing on the same IST(s) since the last relapse were defined as those who previously received IST(s) other than only corticosteroids alone and were continuing on the same IST(s) on which they had the most recent relapse at the time of randomization. Patients who had dose adjustment with the same IST(s) following a relapse were included in this group, e.g., AZA 1.5 mg/kg/day prior to relapse to AZA and 2.0 mg/kg/day after relapse.

1.5.4. Patients with Changes in IST(s) at the Time of Randomization

Patients with changes in IST(s) since the last relapse were defined as those who at the time of randomization, had switched IST (e.g., AZA to MMF), added another IST (e.g., added a corticosteroid), or withdrew any IST(s) following the treatment of the last relapse.

### 1.5.5. The Treating Physician

The Treating Physician was the PI or the Sub-Investigator for the study who was responsible for the overall patient management including patient eligibility evaluation, supervision of the blinded study drug administration, recording and treating of AEs and monitoring of safety assessment. At the time of a relapse, the Treating Physician performed a complete neurologic exam and determined if a patient experienced an On-Trial Relapse and treated the patient's relapse according to the recommended standardized On-Trial Relapse Treatment regimen. Treatment for an On-Trial Relapse was at the discretion of the Treating Physician. The Treating Physician was blinded to patient's treatment assignment.

### 1.5.6. The Examining (Blinded) Physician

The Examining Physician was responsible for performing the EDSS assessments throughout the trial including at the time of a relapse. The Examining Physician performed a complete neurologic exam and documented the FSS and the EDSS score. The Examining Physicians were not the PI or the Treating Physician, and were directly involved in the trial patient's management. The Examining Physician remained blinded to all other trial data as well as all other patient clinical chart data.

Table 3 provides roles and responsibilities of Treating Physician and Examining Physician.

TABLE 3
Roles and Responsibilities of the Treating and the Examining Physicians

| Treating Physician | Examining Physician |
|---|---|
| Blinded to the patient's treatment | Blinded to all other trial data as well |
| assignment: | as all other patient clinical chart data: |
| Determine patient eligibility | At protocol specified time points: |
| for the trial | Complete neurologic examination |
| Overall patient management during | Document FSS scores |
| the trial, including study drug | Record EDSS score |
| administration and safety | |
| assessments. | |
| Perform mRS | |
| Perform C-SSRS* | |
| At the time of relapse: | At the time of relapse: |
| Initial patient assessment | Perform a complete neurologic |
| Have the Examining Physician | examination |
| record FSS scores and EDSS score* | Document FSS scores |
| Perform a complete neurologic | Record EDSS score |
| examination | |
| Determine if the patient has | |
| experienced an On-Trial Relapse | |
| Determine relapse severity by OSIS | |
| Assess visual acuity, Snellen Chart* | |
| Assess ambulation by HAI* | |
| Have the patient complete the | |
| EQ-5D and SF-36* | |
| Treat relapse | |

*can be performed by the Treating Physician or his/her designee.

### 1.7. Trial Visit Procedures

1.7.1. Screening Visit (within 1-3 weeks prior to Baseline [Visit 2/Day 1])

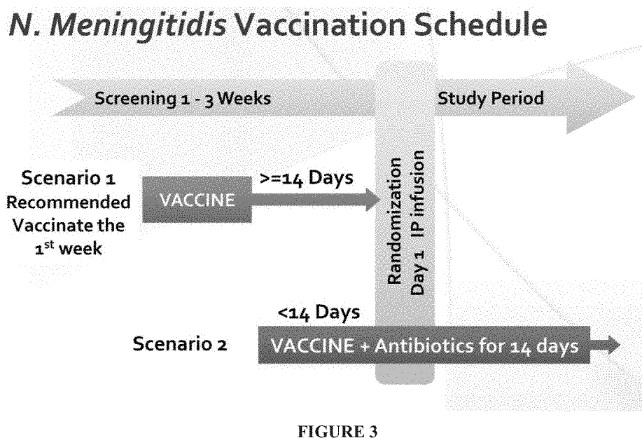

After obtaining written informed consent, the following tests and evaluations were performed within 1-3 weeks prior to randomization at the Baseline Visit (Visit 2/Day 1) to determine patient eligibility for participation in this trial:
Upon ICF signature, register the patient in the IXRS system to get the patient identification number in the study and trigger drug shipment if necessary
Medical History and Demography
NMO History
The Examining Physician performs a complete neurologic examination and FSS to determine the EDSS score
The Treating Physician performed the following:
   Reviewed the signs and symptoms of potential NMO relapse with the patient and instructed the patient to contact the Investigator or his/her designee at the first signs or symptoms of potential relapse. Provided the Patient Education Card describing the potential signs and symptoms of NMO relapse and the contact information of the Investigator
   A complete neurologic examination
The Treating Physician or appropriately trained designee performed the following:
   Visual acuity test (Snellen chart)
   HAI
   Complete Physical Examination including assessments of the following organ/body systems: skin, head, ears, eyes, nose, throat, neck, lymph nodes, pulse, chest, heart, abdomen, extremities, musculoskeletal, and general neurologic examination
   Body weight and height
   Vital signs included assessments of systolic and diastolic blood pressure (BP), temperature, respiration rate (RR), and heart rate (HR)
   Electrocardiogram (ECG)
   Concomitant medications recorded at the Screening Visit including any IST(s) and/or Non-drug therapy for the purpose of relapse prevention or treatment within 24 months prior to screening and all other concomitant medications within 30 days prior to the Screening Visit. If PE was administered within 2 years prior to screening visit, the number of PE cycles for each relapse was collected
   Clinical laboratory tests (chemistry, hematology, and urinalysis)
   Pregnancy test (serum) must be performed on all women of childbearing potential. Note: if the patient was taking/using contraceptive medication/device, please be sure to record the medication or device in the CRF (concomitant medication or procedure)
   Blood sample for NMO-IgG obtained
   If all inclusion criteria and none of the exclusion criteria were met, patients were vaccinated against N. meningitidis (if not already vaccinated within the time period of active coverage specified by the vaccine manufacturer). Patients must be vaccinated at least 14 days prior to receiving the first dose of study medication or be vaccinated and received treatment with appropriate antibiotics until 14 days after the vaccination (FIG. 3).

### 1.7.2. Study Period

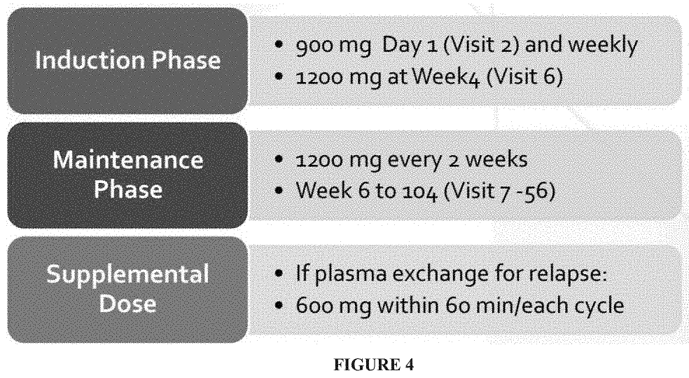

The study period is summarized in FIG. 4. Visit intervals during Induction Phase (Visits 2, 3, 4, 5 and 6) were weekly (every 7±2 days after the last visit). Visit intervals during the Maintenance Phase (Visits 7-56) were every two weeks (every 14 days±2 days after the last visit). Patients who failed to return for a scheduled visit were contacted by the site study staff to determine the reason for missing the appointment, and this information was recorded in the source documents. Patients were strongly encouraged to return to the investigational site for evaluation if a relapse or AE was suspected to have occurred.

1.7.2.1. Induction Phase (Baseline [Visit 2/Day 1] until Visit 6 [Week 4])

### 1.7.2.1.1. Baseline (Visit 2/Day 1)

After the Baseline Visit procedures were performed and the eligibility criteria were confirmed by the Treating Physician (or the PI) and by the Sponsor's Medical Monitor, the patient was randomized on Day 1. Randomization was done by using an interactive voice or web response system (IXRS). The following tests and procedures were completed at the Baseline Visit (Visit 2/Day 1):
The patient completed the self-assessment questionnaires to evaluate quality of life (EQ-5D and SF-36)
The Examining Physician performed a complete neurologic examination and FSS to determine the EDSS
The Treating Physician performed the following assessments:
   Reviewed and assessed the patient for potential signs or symptoms indicative of relapse. Ensured the patient had the Patient Education Card and reminded the patient to contact the Investigator at the first signs or symptoms of potential relapse
   A complete neurologic examination
   Document the OSIS
   mRS
The Treating Physician or appropriately trained designee performed the following:
   Visual acuity test (Snellen chart)
   HAI
   C-SSRS
   Vital signs including assessments of systolic and diastolic BP, temperature, RR and HR
   Any new medications or changes to concomitant medications were recorded
   AEs since the previous visit were evaluated and recorded
   Clinical laboratory tests (chemistry, hematology and urinalysis)
   Pregnancy test (serum) performed on women of childbearing potential
   Collected blood samples for NMO-IgG
   Collected baseline blood samples for PK, PD, free C5 and HAHA assays before the IP infusion
For those patients who had consented, performed lumbar puncture to collect baseline CSF samples for NMO-IgG, PK and free C5 assays before the IP infusion
Instructed the patient on the signs and symptoms of N. meningitdis
Provided the Patient Safety Identification Card describing the IP and emergency contact information to the patient prior to the first dose of IP
Randomized the patient using an interactive voice or web response system (IXRS)
IP (3 vials) was administered and patients were observed for 1 hour following the end of the IP infusion
Collected peak blood samples for PK, PD, and free C5 assays at least 60 minutes after completing the IP infusion

### 1.7.2.1.2. Visits 3-5 (Weeks 1-3)

The Treating Physician or appropriately trained designee reviewed and assessed the patient for any potential signs or symptoms indicative of relapse. The Treating Physician or appropriately trained designee ensured the patient had the Patient Education Card and reminded the patient to contact the Investigator at the first signs or symptoms of potential relapse, including the following:
Vital signs including assessments of systolic and diastolic BP, temperature, RR, and HR
Any new medications or changes to concomitant medications were recorded
Any new AEs or changes in AEs since the previous visit were evaluated and recorded
Ensured that the patient had the Patient Safety Identification Card describing the IP and emergency contact information
Obtained study drug kit assignation through the interactive voice or web response system (IXRS)
IP (3 vials) was administered and patients were observed for 1 hour following the end of the IP infusion

### 1.7.2.1.3. Visit 6 (Week 4)

The following tests and procedures were completed at this visit:
Questionnaires to evaluate quality of life (EQ-5D and SF-36)
The Examining Physician performed a complete neurologic examination and FSS to determine the EDSS
The Treating Physician performed the following assessments:
   Reviewed and assessed the patient for any potential signs or symptoms indicative of relapse
   Ensured the patient had the Patient Education Card and reminded the patient to contact the Investigator at the first signs or symptoms of potential relapse
   A complete neurologic examination
   mRS
The Treating Physician or appropriately trained designee performed the following:
   Visual acuity test (Snellen chart)
   HAI
   C-SSRS
   Vital signs included assessments of systolic and diastolic BP, temperature, RR, and HR
   Any new medications or changes to concomitant medications were recorded
   Any new AEs or changes in AEs since the previous visit were evaluated and recorded
   Ensured that the patient had the Patient Safety Identification Card describing the IP and emergency contact information
   Clinical laboratory tests (chemistry, hematology and urinalysis)
   Collected blood samples for NMO-IgG
   Collected trough blood samples for PK, PD, free C5 and HAHA assays before the IP infusion
   Obtained study drug kit assignation through the interactive voice or web response system (IXRS)
   IP (4 vials) was administered and patients were observed for 1 hour following the end of the IP infusion
   Collected peak blood samples for PK, PD, and free C5 assays 60 minutes after completing the IP infusion
      1.7.2.2. Maintenance Phase (Visit 7 [Week 6] until End of Study or Early Termination Visit)

Patients returned for IP infusions every two weeks (14±2 days) during the Maintenance Phase. The following tests and procedures were completed at every visit beginning at Visit 7 (Week 6) and continuing until the EOS or ET:
The Treating Physician or appropriately trained designee reviews and assessed the patient for any potential signs or symptoms indicative of relapse. Ensured the patient had the Patient Education Card and reminded the patient to contact the Investigator at the first signs or symptoms of potential relapse.
Vital signs included assessments of systolic and diastolic BP, temperature, RR and HR
Any new medications or changes to concomitant medications were recorded
Any new AEs or changes in AEs since the previous visit were evaluated and recorded
Ensured that the patient had the Patient Safety Identification Card describing the IP and emergency contact information
Obtained study drug kit assignation through the interactive voice or web response system (IXRS)
IP (4 vials) was administered and patients were observed for 1 hour following the end of the IP infusion

At Visit 8 (Week 8), Visit 10 (Week 12), Visit 16 (Week 24), Visit 22 (Week 36), Visit 28 (Week 48), Visit 34 (Week 60), Visit 40 (Week 72), Visit 46 (Week 84), Visit 52 (Week 96) and Visit 56 (Week 104), the following procedures were completed (including at the EOS or ET):
The visit schedule was expanded out to at least 144 weeks or more (e.g., 3 to 4 years) prior to entering the extension trial
The patient completed the self-assessment questionnaires to evaluate their quality of life (EQ-5D and SF-36)
The Examining Physician performed a complete neurologic examination and FSS to determine the EDSS score
The Treating Physician performed the following assessments:
   Reviewed and assessed the patient for any potential signs or symptoms indicative of relapse
   Ensured the patient had the Patient Education Card and reminded the patient to contact the Investigator at the first signs or symptoms of potential relapse
   A complete neurologic examination
   mRS
The Treating Physician or appropriately trained designee performed the following:
   Visual acuity test (Snellen chart)
   HAI
   C-SSRS
   Body weight measured at Visits 30 and 56 or EOS/ET Visit
   ECG is performed at Visits 30 and 56 or EOS/ET Visit
   Complete Physical Examination including assessments of the following organ/body systems: skin, head, ears, eyes, nose, throat, neck, lymph nodes, pulse, chest, heart, abdomen, extremities, musculoskeletal, and general neurologic examination at Visit 56 or EOS/ET Visit
   Clinical laboratory tests (chemistry, hematology, and urinalysis)
   Pregnancy test performed on all women of childbearing potential
   Blood samples for NMO-IgG
   Collected trough samples for PK, PD, free C5 and HAHA assays 5-90 minutes before the IP infusion. Note: HAHA will not be collected at Visit 8.
   For those patients who had consented, performed lumbar puncture to collect CSF for NMO-IgG, PK and free C5 assays before IP infusion at Visits 10, 16, 28, 40, 52, 56, and/or EOS/ET Visit
   Collected peak blood samples for PK, PD, and free C5 assays at least 60 minutes after completing the IP infusion

### 1.7.3. Relapse Evaluation

Patients were instructed to contact the Investigator or the appropriate designee at the first sign or symptom of a potential relapse, including the following criteria: new onset of neurologic symptoms or worsening of existing neurologic symptoms with an objective change on neurologic examination that persists for greater than 24 hours; signs and symptoms attributable to NMOSD rather than other causes and onset preceded by ≥30 days of clinical stability.

Patients were ideally evaluated within 24 hours of notification of the Investigator of a possible relapse, and no later than 48 hours. All potential relapses were evaluated by both the Examining and the Treating Physicians.

Follow-up Relapse Evaluation Visits were performed at 1, 4 and 6 weeks after the onset of relapse. Additional (Unscheduled) Relapse Evaluation Visits were permitted at the discretion of the Investigator.

1.7.3.1. Relapse Evaluation Visit (Within 24-48 Hours)

The following tests and procedures were performed at the Relapse Evaluation Visit:
The Examining Physician performed a complete neurologic examination and FSS to determine the EDSS Score
The Treating Physician performed the following assessments:
   A complete neurologic examination
   OSIS
The Treating Physician or appropriately trained designee performed the following:
   Visual acuity test (Snellen chart)
   HAI
   Vital signs included assessments of systolic and diastolic BP, temperature, RR and HR
   Any new medications or changes to concomitant medications were recorded
   Any new AEs or changes in AEs since the previous visit were evaluated and recorded
   Clinical laboratory tests (chemistry, hematology, and urinalysis)
   Collected blood samples for NMO-IgG
   Performed lumbar puncture to collect CSF sample for NMO-IgG, PK, and free C5 assays
If medically indicated for evaluation of relapse, additional tests (e.g., MRI, CT scan, laboratory tests, etc.) were performed at the discretion of the Investigator. If additional medically indicated tests/procedures were performed, the results were recorded on the CRFs.
The Treating Physician determined if the clinical signs, symptoms, and neurologic change (objective findings on the examination) met the definition for On-Trial Relapse as outlined in this protocol
After all specified relapse procedures were complete, the recommended treatment regimen for confirmed relapse outlined in this protocol was initiated at the discretion of the Treating Physician.
IP administration:
   Patients continued IP administration in accordance with protocol-specified IP administration schedule, i.e., every week (7 days±2 days) during induction phase and every two weeks (14 days±2 days) during maintenance phase
   Extra dose (supplemental dose) administered if patients undergo PE. A supplemental dose (2 vials IP) was administered within 60 minutes after each PE session. If PE was administered on the day of regular scheduled IP administration per protocol schedule, patients received the regular scheduled number of vials (3 vials on Visits 2-5, 4 vials on all other Visits) within 60 minutes after each PE session.
   Obtained study drug kit assignation through the interactive voice or web response system (IXRS)
PK, PD sampling:
   Collected one blood sample for PK, PD, and free C5 (if no IP administration at this visit)
   If only IP is administered at the evaluation visit according to the regular IP administration schedule, two blood samples, trough and peak, for PK, PD, and free C5 were collected at: [1] 5-90 minutes before the IP infusion and [2] peak sample at least 60 minutes after the completion of the IP infusion
   If the patient received PE and IP infusion, three blood samples for PK, PD and free C5 collection should have occurred: [1] 5-90 minutes prior to PE [2] at least 60 minutes after PE and 5-90 minutes before IP infusion, and [3] at least 60 minutes after the completion of IP infusion
      1.7.3.2. Follow-Up Relapse Evaluation Visits (Weeks 1, 4 and 6 after relapse)

Follow-up On-Trial Relapse Evaluation Visits were performed at 1, 4 and 6 weeks after the onset of relapse. This trial was a time-to-event trial. Patients were discontinued from this trial and may enter the extension trial (separate protocol) after completion of the Week 6 Relapse Evaluation Visit. Week 6 Relapse Evaluation served as the EOS visit, all procedures listed under the EOS visit were performed. Patients may enter the extension trial (separate protocol) once the Week 6 Relapse Evaluation Visit has been completed and no later than 2 weeks after the last IP administration. This is to ensure no interruption in IP dosing.

After the 6-week follow-up relapse evaluation visit, the data for the patient were forwarded to the RAC for adjudicated relapse determination, which was the primary endpoint. RAC was independent and masked to treatment assignment. All relapses reviewed retrospectively and decisions based on expert opinion of the group.

The following tests and procedures were to have been completed at these Relapse Evaluation visits:
The Examining Physician performed a complete neurologic examination and FSS to determine the EDSS Score
The Treating Physician performed the following:
   A complete neurologic examination
   OSIS
The Treating Physician or appropriately trained designee performed the following:
   Visual acuity test (Snellen chart)
   HAI
   mRS—Week 6 only
   C-SSRS—Week 6 only
   Vital signs included assessments of systolic and diastolic BP, temperature, RR and HR
   Any new medications or changes to concomitant medications were recorded
   Any new AEs or changes in AEs since the previous visit were evaluated and recorded
   Patient self-assessments questionnaires to evaluate quality of life (EQ-5D and SF-36)
Week 6 only
   Complete Physical Examination including assessments of the following organ/body systems: skin, head, ears, eyes, nose, throat, neck, lymph nodes, pulse, chest, heart, abdomen, extremities, musculoskeletal, and general neurologic examination at Week 6 Relapse Evaluation Visit
   Clinical laboratory tests (chemistry, hematology and urinalysis)—Week 6 only
   Collected blood sample for HAHA analysis—Week 6 only
   Pregnancy test performed on all women of childbearing potential—Week 6 only
   For those patients who had consented, performed lumbar puncture to collect CSF for NMO-IgG, PK, and free C5 assays—Week 6 only
If medically indicated for evaluation of relapse, additional tests (e.g., MRI, CT scan, laboratory tests, etc.) might have been performed at the discretion of the Investigator
IP administration during the relapse evaluation period:
   Patients continued IP administration in accordance with protocol specified IP administration schedule, i.e., every week (7 days±2 days) during induction phase and every two weeks (14 days±2 days) during maintenance phase
   Extra doses (supplemental doses) administered if patients undergo PE. A supplemental dose (2 vials IP) was administered within 60 minutes after each PE session. If PE was administered on day of regular scheduled IP administration per protocol schedule, patients received the regular scheduled number of vials IP (3 vials on Visits 2-5, 4 vials on all other Visits) within 60 minutes after each PE session.
   Obtained study drug kit assignation through the interactive voice or web response system (IXRS)
PK, PD sampling during relapse evaluation follow up period:
   Collected one blood sample for PK, PD, and free C5 (if no IP administration at the Relapse Evaluation Follow-up Visits)
   If IP was administered at this evaluation visit according to the regular IP administration schedule, two blood samples, trough and peak, for PK, PD, and free C5 were collected at: [1] 5-90 minutes before the IP infusion and [2] at least 60 minutes after the completion of the IP infusion
   If the patient received PE and IP infusion, three blood samples for PK, PD and free C5 were collected at: [1] 5-90 minutes prior to PE [2] 60 minutes after PE and 5-90 minutes before IP infusion, and [3] at least 60 minutes after the completion of IP infusion

Note: Trough and peak blood samples for PK, PD, and free C5 were collected at the IP administration visits, if per protocol scheduled IP administration did not coincide with the relapse evaluation visit. Vitals signs, information on concomitant medication and AE were collected at the IP administration visit.

### 1.8. Number of Patients

Patient populations were N=47 for placebo and N=96 for eculizumab. NMO patients were randomized in a roughly 2:1 (eculizumab: placebo) ratio at approximately 100-120 centers. Randomization was across centers and stratified by two variables: 1) EDSS score at randomization (Day 1) and 2) patients' IST status at randomization (Day 1). All patients remained on assigned double-blind treatment and background treatment regimen until EOS/ET visit.

### 2. Selection and Withdrawal of Patients

### 2.1. Patient Inclusion Criteria

1. Male or female patients ≥18 years old.
2. Diagnosis of NMO as above.
3. NMO-IgG seropositive at Screening Visit.
4. Historical Relapse (as defined by this protocol) of at least 2 relapses in last 12 months or 3 relapses in the last 24 months with at least 1 relapse in the 12 months prior to the Screening.
5. EDSS score ≤7.
6. If a patient entered the trial receiving an IST, the patient must have been on a stable maintenance dose of IST(s), as defined by the Treating Physician, prior to the screening and must remain on that dose for the duration of the study, unless the patient experienced a relapse (i.e., after the primary efficacy end point for the study has been observed for that patient).
7. Patients must give written informed consent.
8. Patients must be willing and able to comply with the protocol requirements for the duration of the trial.
9. Female patients of child-bearing potential must have a negative pregnancy test (serum HCG). Patients must practice an effective, reliable and medically approved contraceptive regimen during the trial and for up to five months following discontinuation of treatment.

### 2.2. Patient Exclusion Criteria

1. Use of rituximab three months prior to Screening.
2. Use of mitoxantrone three months prior to Screening.
3. Use of IVIg within three weeks prior to screening.
4. If a patient entered the trial receiving oral corticosteroid(s) with or without other IST(s), the daily corticosteroid dose must be no more than prednisone 20 mg/day (or equivalent) prior to the Screening, and must remain on that dose for the duration of the study or until the patient experiences a relapse.
5. Pregnant, breastfeeding, or intending to conceive during the course of the trial.
6. Unresolved meningococcal disease.
7. Any systemic bacterial or other infection that was clinically significant in the opinion of the Investigator and had not been treated with appropriate antibiotics.
8. Participation in any other investigational drug study or exposure to an investigational drug or device within 30 days of screening.
9. Has previously received treatment with eculizumab.
10. Hypersensitivity to murine proteins or to one of the excipients of eculizumab.
11. Any medical condition that, in the opinion of the Investigator, might interfere with the patient's participation in the trial, posed any added risk for the patient, or confounds the assessment of the patients.

### 2.3. Patient Withdrawal Criteria

2.3.1. Withdrawal of Patients from the Trial

Patients were allowed to withdraw consent at any time. Efforts were made to ensure patients were willing to comply with trial participation prior to conducting the screening procedures, and the patients were fully informed of the restrictions related to the change of concomitant medications during the trial. Investigators were able to discontinue a patient's treatment because of AEs, as well as conditions or illnesses that preclude compliance with the protocol from the standpoint of the patient's safety or well-being. All patients of childbearing potential were required to continue using adequate contraception for up to five months following discontinuation of eculizumab treatment.

### 2.4 Patient Characteristics

### 2.4.1 Baseline Characteristics: Demographics

Patients were characterized by baseline Age at First dose, Sex, Race and Region. These demographic characteristics were further characterized in placebo and eculizumab treated groups, and are described in Table 4 below.

TABLE 4
Subject Demographics at Baseline

| | | Placebo | | Eculizumab | | Total | |
|---|---|---|---|---|---|---|---|
| Variable | Statistic | (N = 47) | | (N = 98) | | (N = 143) | |
| Age at First Dose | Mean (SD) | 45.0 | (13.29) | 43.9 | (13.32) | 44.3 | (13.27) |
| (Years) | Media | 44.0 | | 45.0 | | 45.0 | |
| | Min, Max | 21, 75 | | 19, 70 | | 19, 75 | |
| Sex | | | | | | | |
| Males | n (%) | 5 | (10.6) | 8 | (8.3) | 13 | (9.1) |
| Females | n (%) | 42 | (89.4) | 68 | (91.7) | 130 | (90.9) |
| Race | | | | | | | |
| Asian | n (%) | 15 | (31.9) | 37 | (38.5) | 52 | (36.4) |
| Japanese Patients | n (%) | 5 | (10.6) | 9 | (9.4) | 14 | (9.8) |
| Black or African Am. | n (%) | 8 | (17.0) | 9 | (9.4) | 17 | (11.9) |
| White | n (%) | 24 | (51.1) | 46 | (47.9) | 70 | (49.0) |
| Other | n (%) | 0 | (0) | 4 | (42) | 4 | (2.8) |
| Region | | | | | | | |
| Americans | n (%) | 15 | (31.9) | 29 | (30.2) | 44 | (30.8) |
| Europe | n (%) | 19 | (40.4) | 32 | (33.3) | 51 | (35.7) |
| Asia-Pacific | n (%) | 13 | (27.7) | 35 | (36.5) | 48 | (33.6) |

Other Race: 1 American Indian or Alaskan Native, 2 Unknown, 1 Other Americas: Argentina and United States of America: Europe: Czech Republic, Germany, Denmark, Spain, United Kingdom, Ctoatia, Italy, Russia, and Turkey: Asia-Pacific: Australia, Hong Kong, Japan, Korea, Malaysia, Thailand, and Taiwan

### 2.4.2 Baseline Neuromyelitis Optica Spectrum Disorders (NMOSD) Disease Characteristics

Patients were characterized by NMOSD baseline characteristics. These characteristics included the age at clinical presentation of NMOSD, time (years) from initial clinical presentation to first dose, total number of relapses per patient within the 24 months prior to screening, patient historical Annualized Relapse Rate (ARR) within 24 months prior to screening, as well as baseline evaluations including baseline EDSS score, baseline HAI score and baseline mRS score. Eculizumab and placebo treatment arms were similarly characterized. These characteristics are described in Table 5 below. Most (90.9%) of the study population were women. At baseline, the mean (±standard deviation) annualized relapse rate during the previous 24 months was 1.99±0.94, and the median scores on the EDSS, mRS, and HAI indicated moderate-to-severe disability.

TABLE 5
Baseline NMOSD Disease Characteristics

| | | Placebo | Eculizumab | Total |
|---|---|---|---|---|
| Variable | Statistic | (N = 47) | (N = 96) | (N = 143) |
| Age at Initial Clinical | Mean (SD) | 38.5 (14.98) | 35.8 (14.03) | 36.6 (14.35) |
| Presentation | Median | 38.0 | 35.5 | 36.0 |
| | Min, Max | 12, 73 | 5, 66 | 5, 73 |
| Time from Initial Clinical | Mean (SD) | 6.60 (6.586) | 8.16 (8.579) | 7.65 (7.989) |
| Presentation to First Dose | Median | 3.76 | 5.03 | 4.80 |
| (years) | Min, Max | 0.5, 29.1 | 0.4, 44.9 | 0.4, 44.9 |
| Number of Relapses within | Mean (SD) | 3.2 (0.97) | 3.3 (1.13) | 3.3 (1.08) |
| the 24 months prior to | Median | 3.0 | 3.0 | 3.0 |
| Screening | Min, Max | 2, 6 | 2, 7 | 2, 7 |
| Historical Annualized | Mean (SD) | 2.07 (1.037) | 1.94 (0.896) | 1.99 (0.943) |
| Relapse Rate within 24 | Median | 1.92 | 1.85 | 1.92 |
| months prior to Screening | Min, Max | 1.0, 6.4 | 1.0, 5.7 | 1.0, 6.4 |
| Baseline EDSS Score | Mean (SD) | 4.26 (1.510) | 4.15 (1.646) | 4.18 (1.598) |
| | Median | 4.00 | 4.00 | 4.00 |
| | Min, Max | 1.0, 6.5 | 1.0, 7.0 | 1.0, 7.0 |
| Baseline HAI Score | Mean (SD) | 2.1 (1.40) | 2.4 (2.17) | 2.3 (1.95) |
| | Median | 2.0 | 2.0 | 2.0 |
| | Min, Max | 0, 6 | 0, 8 | 0, 8 |
| Baseline mRS Score | Mean (SD) | 2.1 (0.98) | 2.1 (1.14) | 2.1 (1.09) |
| | Median | 2.0 | 2.0 | 2.0 |
| | Min, Max | 0, 4 | 0, 4 | 0, 4 |

Female patients were further characterized by NMOSD baseline characteristics, as shown in Table 6 below.

TABLE 6
Baseline NMOSD Disease Characteristics of Female Subject

| | Eculizumab | | Placebo | |
|---|---|---|---|---|
| Characteristic | (n = 96) | | (n = 47) | |
| Female sex, n (%) | 88 | (92) | 42 | (89) |
| Mean (SD) age, years | | | | |
| At first dose of trial medication | 43.9 | (13.32) | 45.0 | (13.29) |
| At initial clinical presentation | 35.8 | (14.03) | 38.5 | (14.98) |
| Diagnosis, n (%) | | | | |
| Neuromyelitis optica | 69 | (72) | 38 | (81) |
| NMOSD | 27 | (28) | 9 | (19) |
| Mean (SD) ARR in previous 24 months | 1.94 | (0.90) | 2.07 | (1.04) |
| Median (range) EDSS score | 4.00 | (1.0-7.0) | 4.00 | (1.0-6.5) |
| ISTs at baseline, n (%) | | | | |
| None | 21 | (22) | 13 | (28) |
| Corticosteroids one | 16 | (17) | 11 | (23) |
| Azathioprine ± corticosteroids | 37 | (39) | 13 | (28) |
| Mycophenolate mofetil ± corticosteroids | 17 | (18) | 8 | (17) |
| Other drug^{a} ± corticosteroids | 5 | (5) | 2 | (4) |
| Previous rituximab treatment,^{b} n (%) | 26 | (27) | 20 | (43) |

### 2.4.3 Baseline Supportive IST Use at Baseline

Patients were characterized by supportive IST use prior to screening. Patients were characterized as having no IST usage, having received steroids alone, having supportive IST that consisted of AZA alone or AZA with steroids, MMF alone or MMF with steroids, as well as a group receiving other IST alone or other IST with steroids. These patient characteristics are summarized in Table 7 below. Additional IST use and history of an autoimmune disorder is summarized in Table 36.

TABLE 7
Supportive IST Use at Baseline

| | | Placebo | Eculizumab | Total |
|---|---|---|---|---|
| Variable | Statistic | (N = 47) | (N = 96) | (N = 143) |
| No IST Usage | n (%) | 13 (27.7) | 21 (21.9) | 34 (23.8) |
| Steroids Alone | n (%) | 11 (23.4) | 16 (16.7) | 27 (18.9) |
| AZA Subgroup | | 13 (27.7) | 37 (38.5) | 50 (35.0) |
| AZA Alone | n (%) | 6 (12.8) | 8 (8.3) | 14 (9.8) |
| AZA + steroids | n (%) | 7 (14.9) | 29 (30.2) | 36 (25.2) |
| MMF Subgroup | | 8 (17.0) | 17 (17.7) | 25 (17.5) |
| MMF Alone | n (%) | 5 (10.6) | 10 (10.4) | 15 (10.5) |
| MMF + steroids | n (%) | 3 (6.4) | 7 (7.3) | 10 (7.0) |
| Other IST(s) | | 2 (4.3) | 5 (5.2) | 7 (4.9) |
| Other IST(s) Alone | n (%) | 0 (0.0) | 1 (1.0) | 1 (0.7) |
| Other IST(s) + steroids | n (%) | 2 (4.3) | 4 (4.2) | 6 (4.2) |

Note:
Use of Rituximab was not allowed 3 months prior to screening. 46 (32.2%) had previously received Rituximab for NMOSD (26 emilizumab and 20 placebo).

### 3. Treatment of Patients

### 3.1. Investigational Product Dosage and Administration

Eculizumab (600 mg, 900 mg, or 1200 mg) or matching placebo was administered IV over approximately 35 minutes according to the regimen in Table 8. Treatment continued until a patient experienced a physician-determined relapse (irrespective of the outcome of subsequent adjudication), discontinued, or until the trial ended. A greater proportion of patients receiving eculizumab (16.7%) than placebo (6.4%) discontinued from the study.

TABLE 8
Investigational Product Dosage and Administration

| | | | | Equivalent |
|---|---|---|---|---|
| | Frequency of Investigational Product (IP) | Visit | # of | Eculizumab |
| Dose Period | Administration | # | Vials | Dose |
| Induction Phase | Weekly (every 7 ± 2 days) | 2-5 | 3 | 900 mg |
| | | 6 | 4 | 1200 mg |
| Maintenance Phase | Every 2 weeks (14 ± 2 days) from the | 7-56 | 4 | 1200 mg |
| | fifth dose onward | | | |
| Supplement Doses* | If PE is given for On-Trial Relapse, | | 2 | 600 mg |
| | administer within 60 minutes after each PE as | | | |
| | described below*. | | | |

Induction Phase

Eculizumab or placebo: 3 vials of IP (equivalent to 900 mg of eculizumab) weekly×4 (every 7 days±2 days) followed by 4 vials of IP (equivalent to 1200 mg of eculizumab) one week later for the fifth dose (Visit 6/Week 6).

Maintenance Phase

Eculizumab or placebo: 4 vials of IP (equivalent to 1200 mg of eculizumab) every two weeks (14 days±2 days).

Supplemental Doses

If patient had undergone PE for On-Trial Relapse on a day that IP administration was not routinely scheduled during the Study Period, a supplemental dose (2 vials IP; equivalent to 600 mg of eculizumab or matching placebo) should have been administered within 60 minutes after each PE. If PE was administered on a day of regularly scheduled IP administration, patients received the regularly scheduled number of vials (3 vials on Visits 2-4; 4 vials on all other Visits) within 60 minutes after each PE. Patients continued protocol specified dosing until the 6-week post-attack assessment.

### 3.2. Concomitant Medications

### 3.2.1. Allowed Medications

### 3.2.1.1. Palliative and Supportive Care

Palliative and supportive care was permitted during the course of the trial for underlying conditions.

### 3.2.1.2. Immunosuppressive Therapy Agents

IST agents, such as corticosteroids, AZA, MMF, methotrexate, tacrolimus, cyclosporine or cyclophosphamide, either in combination or mono-therapy, were permitted. The choice of IST agents was left to the discretion of the Treating Physician with the exception of the disallowed medications. Standard recommended dosing should have been used for the chosen IST. Use of corticosteroids was permitted; however, to assure balance between treatment groups with respect to steroid dosing, the total daily dose was not to exceed prednisone 20 mg per day or equivalent.

If a patient entered the trial receiving IST(s), the patient must have been on a stable maintenance dose of IST(s), as defined by their Treating Physician, prior to the Screening Visit and was required to remain on that dose for the duration of the study. No new IST(s) or switch to another IST was permitted during the trial unless the patient experienced a relapse (i.e., after the primary efficacy endpoint for the study had been observed in that patient). After a relapse there were no restrictions on medication adjustments or changes.

If a patient entered the trial receiving steroids, either as mono-therapy or in combination with another IST, the daily steroid dose could not exceed prednisone 20 mg daily (or equivalent). The patient was required to remain on that dose for the duration of the trial unless the patient experienced a relapse.

The IST(s) and its dosing regimen for a particular patient were required to remain stable during the trial.

### 3.2.2. Disallowed Medications

The following medications were prohibited during the trial:
Concomitant use of rituximab with eculizumab
Mitoxantrone
Immunomodulatory therapies including: interferon beta-lb; interferon beta-la and glatiramer acetate
IVIg for relapse prevention
PE for relapse prevention

### 3.3. Treatment Compliance

Patients were infused IV with IP under the supervision of the PI/Sub-Investigator or their designee to ensure that the patient receives the appropriate dose at the appropriate time-points during the trial.

### 3.4. Randomization and Blinding

### 3.4.1. Randomization

Computer-generated randomization lists were prepared by a third party under the direction of the Sponsor. Investigators, or designees, enrolled patients and obtained randomization codes using an interactive voice/web response system.

Patients were randomized on Day 1 after the Investigator (Treating Physician) and the Sponsor's Medical Monitor verified that they were eligible. A randomization worksheet was provided and the Sponsor's approval was required to ensure that patients are randomized properly. Patients were randomized on a 2:1 basis to the Eculizumab Arm or the Placebo Arm. The randomization and stratification occurred across centers. The randomization stratification included two variables: 1) EDSS score at randomization (Day 1); and 2) patients' prior IST and IST status at the time of randomization (Day 1).
1. EDSS score at randomization (Day 1)
a. EDSS score are ≤2.0
b. EDSS scores are 2.5 to ≤7
2. Patients' IST status at randomization (Day 1)
a. Treatment of naïve patients (i.e., patients with no prior or current ISTs, except steroids alone)
b. Patients continuing on the same IST(s) since the last relapse (i.e., including patients with dose adjustment with the same IST(s) since the last relapse)
c. Patients with changes in IST(s) since last relapse (i.e., switched IST(s) [e.g., AZA to MMF], added IST [e.g., corticosteroid], or withdrew any IST treatment)

Patients were centrally randomized using an interactive voice or web response system (IXRS). All patients were assigned to double-blind treatment by the secure IXRS randomization application. A summary of randomized and stratified patient populations based on EDSS stratification, randomized IST Stratification, and overall stratification groupings could be found in Table 9 below.

TABLE 9
Randomization Stratification

| | Placebo | Eculizuntab | Total |
|---|---|---|---|
| Variable | (N = 47) | (N = 96) | (N = 143) |
| EDSS Stratification, n (%) | | | |
| Low EDSS (<=2.0) | 5 (10.6) | 11 (11.5) | 16 (11.2) |
| High EDSS (>=2.5 to <=7) | 42 (89.4) | 85 (88.5) | 127 (88.8) |
| IST Stratification, n (%) | | | |
| Treatment Naïve | 5 (10.6) | 14 (14.6) | 19 (13.3) |
| Continuing on Same IST(s) Since Last Relapse | 24 (51.1) | 49 (51.0) | 73 (51.0) |
| Changes in IST(s) Since Last Relapse | 18 (38.3) | 33 (34.4) | 51 (35.7) |
| Overall Stratification Groupings (4 strata for analysis), n (%) | | | |
| Low EDSS (<=2.0) | 5 (10.6) | 11 (11.5) | 16 (11.2) |
| High EDSS (>=2.5 to <=7) and Treatment Naïve | 5 (10.6) | 12 (12.5) | 17 (11.9) |
| High EDSS (>=2.5 to <=7) and Continuing on | | | |
| Same IST(s) Since Last Relapse | 22 (46.8) | 44 (45.8) | 66 (46.2) |
| High EDSS (>=2.5 to <=7) and Changes in IST(s) | 15 (31.9) | 29 (30.2) | 44 (30.8) |
| Since Last Relapse | | | |

### 3.4.2. Blinding and Unblinding

All trial patients, investigational site personnel, Sponsor staff, Sponsor designees, and all staff directly associated with the conduct of the trial were blinded to the patient treatment assignments. The double-blind was maintained by using identical IP kits and labels for eculizumab and placebo. The placebo had an identical appearance to that of eculizumab.

There was no antidote to reverse the effects of eculizumab. Therefore, unblinding would not have been helpful in the planning of patient treatment for a given event. Unblinding would only have been considered for the safety of the subject. If, unblinding were deemed to be necessary by the Investigator, the Investigator could unblind the patient's treatment allocation using the interactive voice or web response system (IXRS). In the situation where an unexpected, serious and related AE occurs, the blind was broken by the Sponsor only for that specific subject. The blind would be maintained for persons responsible for the ongoing conduct of the study (such as the management, monitors, investigators, etc.) and those responsible for data analysis and interpretation of results at the conclusion of the study, such as biometrics personnel. Unblinded information was, is, and will only be accessible to those who need to be involved in the safety reporting to Health Authorities, Ethics Committees/IRBs and Data Safety Monitoring Boards (DSMB)/Data Monitoring Committee (DMC), or persons performing ongoing safety evaluations during the trial.

Investigators received only blinded information unless unblinded information would have been judged necessary for safety reasons.

Unblinding took place for one patient who had withdrawn from the study (eculizumab group) and one patient experiencing an adverse event (AE) subsequently leading to withdrawal (placebo group). No cases of Sponsor unblinding took place.

### 4. Investigational Product Materials and Management

### 4.1. Investigational Product

Each vial of IP contained eculizumab 300 mg or matching placebo for IV administration.

### 4.2. Investigational Product Packaging and Labeling

The active IP, eculizumab, was supplied in single 30 mL vials as a solution concentration of 10 mg/mL. The comparator product was manufactured as a matching sterile, clear, colorless solution with the same buffer components but without active ingredient, in an identical 30 mL vial.

All study drugs were prepared in vials, packaged in kits, and labeled in an identical manner.

TABLE 10
Investigational Product

| | Investigational Product | |
|---|---|---|
| Product Name: | Eculizumab | Placebo |
| Dosage Form: | Concentrate for solution | Solution for infusion |
| | for infusion | |
| Unit Dose: | 300 mg | 0 mg |
| Route of Administration: | Intravenous Infusion | Intravenous Infusion |
| Physical Description: | 30 mL vial | 30 mL vial |

### 4.3. Investigational Product Storage

IP was released to each site upon receipt of all required essential documents based upon federal, state, and local regulations. Each kit had a one-panel label describing the contents and a place for the pharmacist to record the patient number and initials.

Upon arrival at the center, the IP was promptly removed from the shipping cooler and stored in refrigerated conditions at 2° C. to 8° C. with minimal light exposure. IP should have been stored in a secure, limited-access storage area.

Diluted solutions of IP could have been stored at 2° C. to 8° C. (36-46° F.) for up to 24 hours prior to administration. If the IP was prepared more than 4 hours in advance of a patient's visit, the diluted material should have been stored at 2° C. to 8° C. The solution should have been allowed to warm to room temperature prior to administration, but not be heated (e.g., by using a microwave or other heat source) other than by ambient air temperature.

### 4.4. Investigational Product Preparation

Infusions of IP should have been prepared using aseptic technique and the dose regimen. The protocol for IP preparation involved withdrawing the required amount of IP from the vials, transferring the recommended dose to an infusion bag, and diluting IP to a final concentration of 5 mg/mL by addition to the infusion bag of the appropriate amount (equal volume) of one of the following: 0.9% sodium chloride injection, USP; 0.45% sodium chloride injection, USP; 5% dextrose in water injection, USP; or Ringer's injection, USP. The final volume of a 5 mg/mL diluted IP solution is 120 mL for 600 mg doses (2 vials), 180 mL for 900 mg doses (3 vials) and 240 mL for 1200 mg doses (4 vials) as shown in Table 11.

TABLE 11
Investigational Product

| | Volume | Volume of | Total Volume of |
|---|---|---|---|
| | of IP | Diluent* | Administration |
| 600 mg (2 vials) | 60 mL | 60 mL | 120 mL |
| 900 mg (3 vials) | 90 mL | 90 mL | 180 mL |
| 1200 mg (4 vials) | 120 mL | 120 mL | 240 mL |

*Choose one of the following diluents: a) 0.9% sodium chloride; b) 0.45% sodium chloride; c) 5% dextrose in water; d) Ringer's injection

The infusion bag containing the diluted IP solution was gently agitated the infusion bag to ensure thorough mixing of the product and diluents and was allowed to warm to room temperature by exposure to ambient air prior to administration.

4.5. Administration

IP was administered via IV infusion. Prior to administration, the diluted solution was allowed to warm to room temperature by exposure to ambient air. Parenteral drug products should have been inspected visually for particulate matter and discoloration prior to administration.

The diluted IP was intravenously administered over 35 minutes (range 25 to 45 minutes). It was not necessary to protect the infusion bags from light while IP is being administered to the patient. At the site's discretion, the diluted IP could have been administered via gravity feed, a syringe-type pump, or an infusion pump. The patients were monitored for 1 hour following infusion.

If an AE occurred during the administration of the IP, the infusion could have been slowed or stopped at the discretion of the Investigator, depending upon the nature and severity of the event. The AE would have been captured in the patient's source document and CRF.

### 5. Assessment of Efficacy

### 5.1. Efficacy Parameters

Treatment commenced with the first IP infusion (eculizumab or placebo). The Study Period i.e., double-blind treatment period, defined the time period for assessment of the trial endpoints. A total of 24 adjudicated relapse events in 24 distinct patients had to be observed. At the point where the trial was stopped (after 24 events), all data from all patients were collected, and the database cleaned, locked, and analyzed. Data from the Study Period was used for efficacy analysis.

### 5.1.1. Relapses

Pre-treatment relapses were reviewed by the Investigator to determine if they meet criteria for Historical Relapse as defined by this protocol. On-Trial Relapses were monitored throughout the trial. Patients were educated on the potential signs and symptoms of NMO relapse, and were monitored throughout by physicians. The Investigator or his/her designee reviewed, in detail, the signs and symptoms of a potential relapse with the patient at each visit. Patients were instructed to contact the investigator or the appropriate designee at the first sign or symptom of a relapse. Patients were to have been evaluated within 24 hours of notification of the investigator or the appropriate designee of a possible attack, and no later than 48 hours.

All potential relapses were evaluated by both the Examining and the Treating Physicians. The Treating Physician made the decision as to whether the clinical signs, symptoms and neurological change (objective findings on exam) met the definition of On-Trial Relapse and treated the patient's relapse according to the recommended Standardized Treatment Plan. The relapse treatment was left to the Treating Physician's discretion. All potential relapses were also forwarded and evaluated by the RAC. To ensure no relapses missed being reported, the RAC also evaluated cases of interest. These involved:
patients who were seen by the treating physician for a 24-48 hour relapse evaluation visit and when the treating physician concluded that an On-Trial Relapse did not occur, OR
a sentinel AE (e.g., AEs of weakness, sensory changes, especially in a dermatomal distribution, characteristic visual changes of NMO, or pseudoexacerbation of NMO) AND either:
   a magnetic resonance imaging scan was performed contemporaneously OR
   neurologic symptoms were treated (by hospitalization or intravenous methylprednisolone for 3 days or fewer) OR
   contemporaneous objective worsening of the treating physician's neurologic examination (defined by answer of ‘worse’ to the question asking about clinically meaningful change from prior examination or answer ‘yes’ to question asking if there had been a clinically meaningful change from the most recent examination).

Individual physicians determined the most appropriate relapse treatment.

### 5.1.2. Disability

Disability was assessed based on the EDSS scores comparing the change from baseline in the two treatment groups. The Kurtzke Expanded Disability Status Scale (EDSS) is a method of quantifying disability in multiple sclerosis (MS). The EDSS replaced the previous Disability Status Scales used in MS. The EDSS quantified disability in eight Functional Systems (FS) and allows neurologists to assign a Functional System Score (FSS) in each of these. The Functional Systems are: pyramidal, cerebellar, brainstem, sensory, bowel and bladder, visual, cerebral, and other. EDSS steps 1.0 to 4.5 refer to people with MS who are fully ambulatory. EDSS steps 5.0 to 9.5 are defined by the impairment of ambulation. These steps are shown in Table 12.

TABLE 12
Kurtzke Expanded Disability Status Scale (EDSS)

| | |
|---|---|
| 0.0 | Normal neurological examination |
| 1.0 | No disability, minimal signs in one FS |
| 1.5 | No disability, minimal signs in more than one FS |
| 2.0 | Minimal disability in one FS |
| 2.5 | Mild disability in one FS or minimal disability in two FS |
| 3.0 | Moderate disability in one FS, or mild disability in three or four FS. Fully ambulatory |
| 3.5 | Fully ambulatory but with moderate disability in one FS and more than minimal disability in several |
| | others |
| 4.0 | Fully ambulatory without aid, self-sufficient, up and about some 12 hours a day despite relatively severe |
| | disability; able to walk without aid or rest some 500 meters |
| 4.5 | Fully ambulatory without aid, up and about much of the day, able to work a full day, may otherwise have |
| | some limiation of full activity or require minimal assistance; characterized by relatively severe disability; |
| | able to walk without aid or rest some 300 meters. |
| 5.0 | Ambulatory without aid or rest for about 200 meters; disability severe enough to impair full daily |
| | activities (work a full day without special provisions) |
| 5.5 | Ambulatory without aid or rest for about 100 meters; disability severe enough to preclude full daily |
| | activities |
| 6.0 | Intermittent or unilateral constant assistance (cane, crutch, brace) required to walk about 100 meters with |
| | or without resting |
| 6.5 | Constant bilateral assistance (canes, crutches, braces) required to walk about 20 meters without resting |
| 7.0 | Unable to walk beyond approximately five meters even with aid, essentially restricted to wheelchair; |
| | wheels self in standard wheelchair and transfers alone; up and about in wheelchair some 12 hours a day |
| 7.5 | Unable to take more than a few steps; restriced to wheelchair; may need aid in transfer; wheels self but |
| | cannot carry on in standard wheelchair a full day; May require motorized wheelchair |
| 8.0 | Essentially restricted to bed or chair or perambulated in wheelchair, but may be out of bed itself much of |
| | the day; retains many self-care functions; generally has effective use of arms |
| 8.5 | Essentially restricted to bed much of day; has some effective use of arms retains some self care functions |
| 9.0 | Confined to bed; can still communicate and eat. |
| 9.5 | Totally helpless bed patient; unable to communicate effectively or eat/swallow |
| 10.0 | Death due to MS |

The Examining Physician who was blinded to all other trial and patient clinical data was responsible for performing the EDSS assessments throughout the trial at the protocol specified time points as well as at the On-Trial Relapse Evaluation Visit.

In addition, disability was assessed based on the mRS score comparing the change from baseline in the two treatment groups. mRS score was assessed by The Treating Physician at the protocol specified time points.

5.1.3. Neurologic Functions Neurologic function (including visual acuity) was assessed by blinded raters based on

EDSS, and by physicians (or trained designees) using the Modified Rankin Scale (mRS, on which scores range from 0 to 523 [or 6, with ‘death’ added]) and the Hauser Ambulation Index (HAI, on which scores range from 0 to 9). Higher scores indicate worse neurologic function or disability in each case. FSS ambulatory function was assessed by HAI scale and visual function is measured by visual acuity using the Snellen chart. In addition, the EDSS visual (optic) functional system score was used for statistical analysis of changes in visual acuity. Neurologic function evaluation was assessed at the protocol specified time points as well as at relapse evaluation visit(s). The HAI scale is provided in Table 13.

TABLE 13
HAI Scale of Ambulatory Function

| | |
|---|---|
| [symbol] | 0 = Asymptomatic; fully active. |
| [symbol] | 1 = Walks normally, but reports fatigue that interferes with athletic or other |
| | demanding activities |
| [symbol] | 2 = Abnormal gait or episodic imbalance; gait disorder is noticed by family and |
| | friends; able to walk 25 feet (8 meters) in 10 seconds or less. |
| [symbol] | 3 = Walks independently; able to walk 25 feet in 20 seconds or less. |
| [symbol] | 4 = Requires unilateral support (cane or single crutch) to walk; walks 25 feet in 20 |
| | seconds or less. |
| [symbol] | 5 = Requires bilateral support (canes, crutches, or walker) and walks 25 feet in 20 |
| | seconds or less; or requires unilateral support but needs more than 20 seconds |
| | to walk 25 feet. |
| [symbol] | 6 = Requires bilateral support and more than 20 seconds to walk 25 feet; may use |
| | wheelchair* on occasion. |
| [symbol] | 7 = Walking limited to several steps with bilateral support; unable to walk 25 feet; |
| | may use wheelchair* for most activities. |
| [symbol] | 8 = Restricted to wheelchair; able to transfer self independently. |
| [symbol] | 9 = Restriced to wheelchair; unable to transfer self independently. |

*The use of a wheelchair may be determined by lifestyle and motivation. It is expected that patients in Grade 7 will use a wheelchair more frequently then those in Grades 5 or 6. Assignment of grade in the range of 5 to 7, however, is determined by the patient's ability to walk a given distance, and not by the extent to which the patient uses a wheelchair.

### 5.1.4. Quality of Life

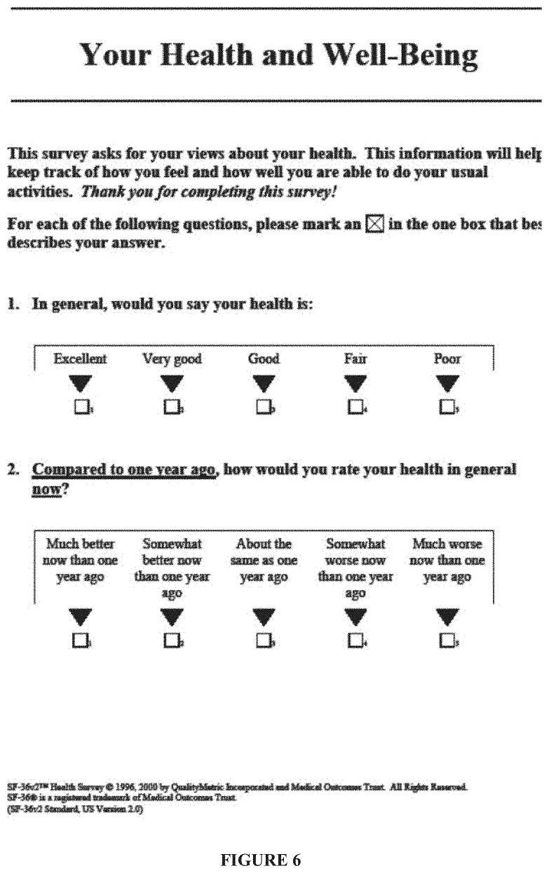

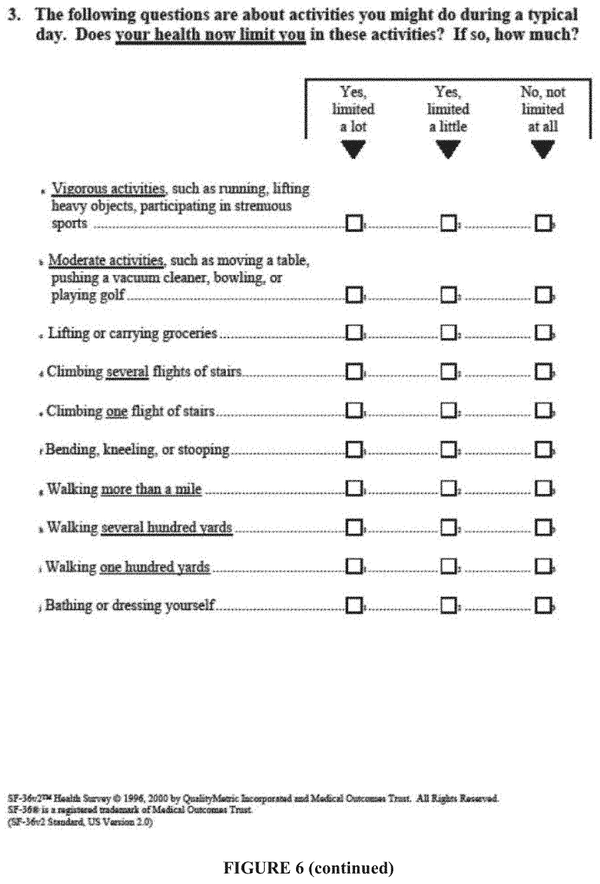

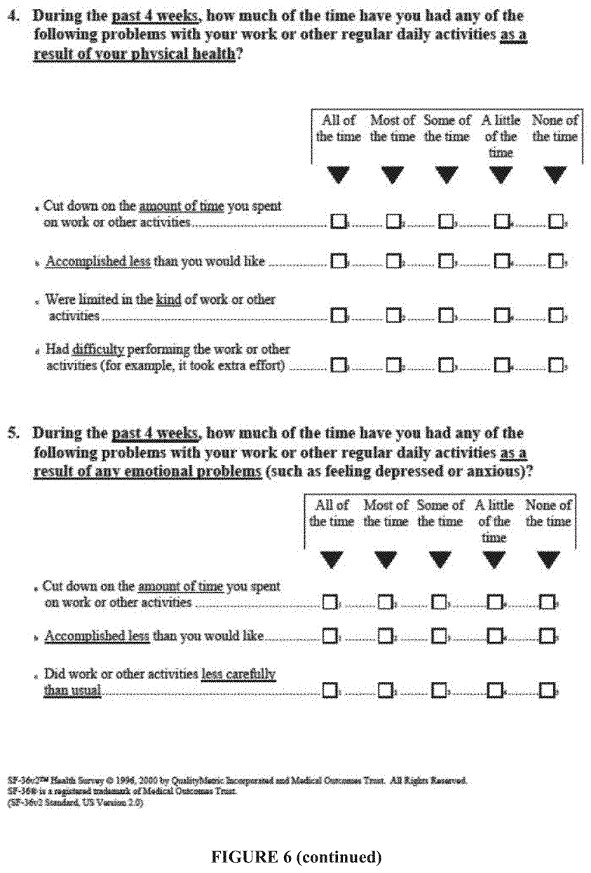

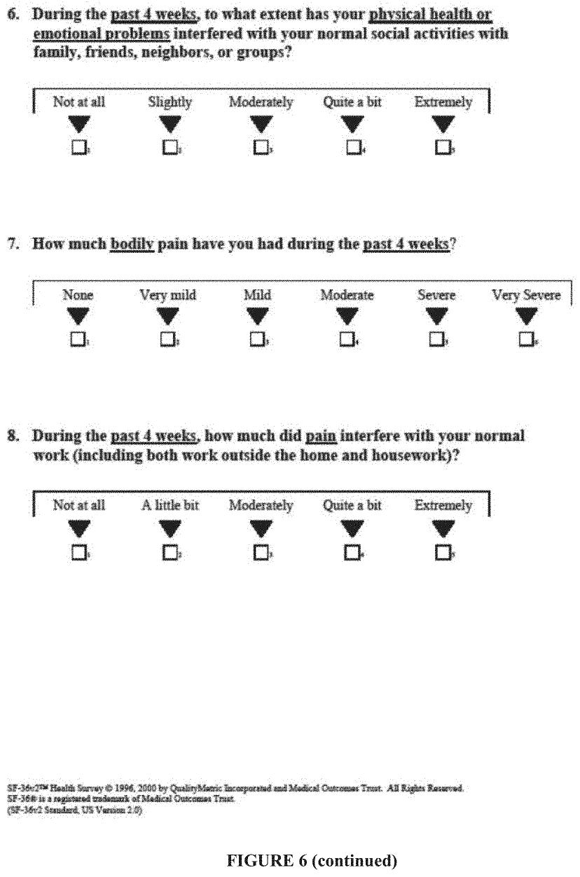

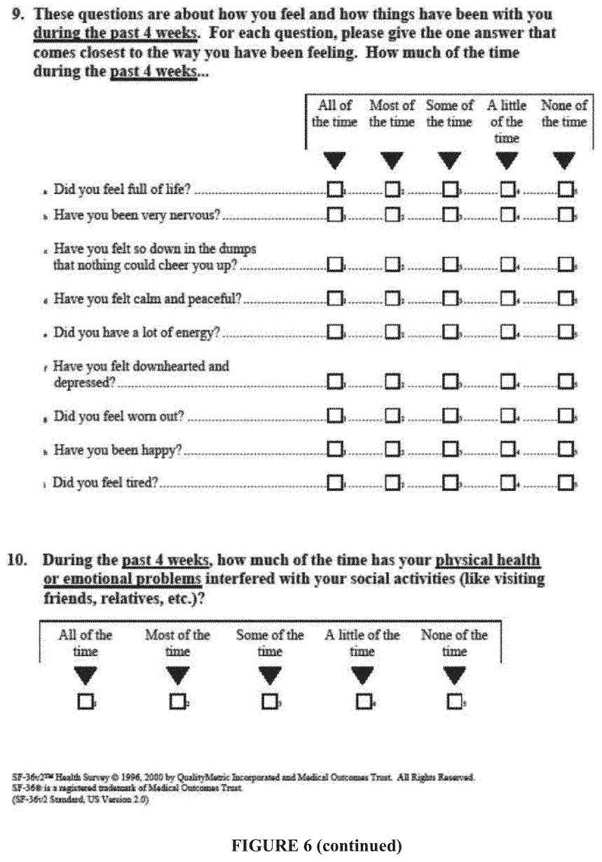

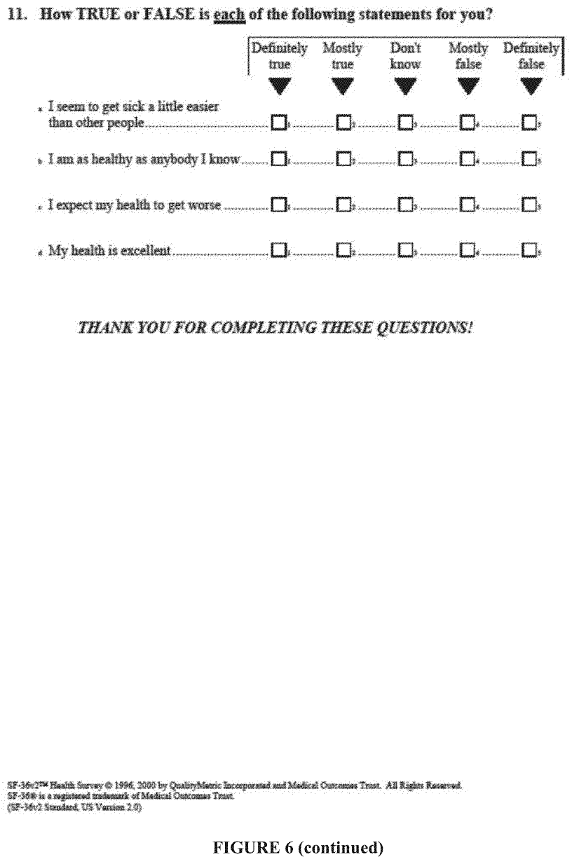

QOL was assessed by the patient self-assessment questionnaires EQ-5D and SF-36 at the protocol specified time points. A sample questionnaire for EQ-5D is shown in FIG. 5, in which visual analog scale [VAS] scores range from 0 to 100, and summary index scores range from <0 to 1 [higher scores represent better health status]. Assessments were performed at baseline, weeks 4, 8, 12, every 12 weeks thereafter, and study end/discontinuation. A sample questionnaire for SF-36 is shown in FIG. 6.

SF-36 scores were analyzed, including physical component score (PCS) and mental component score (MCS). At baseline, 4, 8, 12 and every 12 weeks thereafter until study end/relapse (EOS), patients completed the 36-item short form health survey (SF-36), and scores were assessed using a randomization-based non-parametric analysis of covariance.

Mean (SD) SF-36 scores at baseline were 39.12 (11.15) for eculizumab and 37.63 (11.00) for placebo. From baseline to EOS, mean SF-36 scores improved with eculizumab and worsened with placebo by 2.65 (8.53) and −0.23 (9.38), respectively (p<0.05). At baseline, mean PCS for eculizumab and placebo were 38.6 (9.8) and 36.9 (10.8), respectively, and changed from baseline to EOS by 3.4 (7.7) and 0.7 (8.3), respectively (p=0.02). At baseline, mean MCS for eculizumab and placebo were 47.03 (12.5) and 44.03 (11.4), respectively, and changed from baseline to EOS by 0.45 (10.60) and −0.06 (11.79), respectively (p=0.29).

From baseline to the EOS, SF-36 scores improved in eculizumab-treated patients, yet worsening scores in placebo-treated patients. Changes in the PCS and MCS sub-scales suggest that improvements in QoL observed with SF-36 were driven mainly by physical components and less so by mental components of the survey. Long-term follow-up in the open-label extension phase of PREVENT will help further establish the benefits of eculizumab in patients with NMOSD.

Safety assessments included monitoring for adverse events, vital signs, physical examination, electrocardiography, clinical laboratory tests, and suicidal ideation and behavior. NMOSD relapses were recorded as adverse events if they met the criteria for serious adverse events. Definitions of adverse events are described below.

An AE was the development of an undesirable medical condition or the deterioration of a pre-existing medical condition following or during exposure to a pharmaceutical product, whether or not considered causally related to the product. In clinical studies, an AE could include an undesirable medical condition occurring at any time, including baseline, even if no investigational product has been administered. AEs could be classified into non-serious AEs or serious AEs.

An AE could be any unfavorable and unintended sign (including an abnormal laboratory finding, for example), symptom, or disease temporally associated with the use of a medicinal product, whether or not considered related to the medicinal product.

Any AE that fulfilled one or more of the criteria listed below must be recorded as a serious AE. A serious AE (experience) or reaction was any untoward medical occurrence that at any dose:
results in death
is life-threatening
required inpatient hospitalization or prolongation of existing hospitalization
resulted in persistent or significant disability/incapacity
was a congenital anomaly/birth defect
was an important medical event.

The term ‘life-threatening’ in the definition of ‘serious’ referred to an event in which the patient was at risk of death at the time of the event; it did not refer to an event that hypothetically might have caused death if it were more severe.

Medical and scientific judgment should be exercised in deciding whether expedited reporting was appropriate in other situations, such as important medical events that might not be immediately life-threatening or result in death or hospitalization, but might jeopardize the patient or might require intervention to prevent one of the other outcomes listed in the definition above. These should also usually be considered serious. Examples of such events were: intensive treatment in an emergency room or at home for allergic bronchospasm; blood dyscrasias or convulsions that did not result in hospitalization; and development of drug dependency or drug abuse.

Suicidal Ideation and Behavior: the Columbia-Suicide Severity Rating Scale was administered to ensure that patients who were experiencing suicidal ideation or behavior were properly recognized and adequately managed.

### 6. Statistical Method and Planned Analyses

### 6.1. General Considerations

Prior to locking the database, all data editing was complete and decisions regarding the evaluability of all patient data for inclusion in the statistical analysis for the Per-Protocol (PP) Set were made. The rationale for excluding any data from the statistical analyses was prospectively defined, and classification of all or part of a patient's data as non-evaluable was completed and documented before the database was locked and before the statistical analysis began.

All summary statistics were computed and displayed by treatment group and scheduled assessment time. Summary statistics for continuous variables minimally included n, mean, standard deviation, minimum, median, and maximum. For categorical variables, frequencies and percentages are presented. Graphical displays are provided as appropriate.

Statistical analyses were performed with SAS® software, version 9.4 (SAS Institute, Inc.).

### 6.2. Determination of Sample Size

This is a randomized, double-blind, placebo-controlled trial to evaluate eculizumab in NMO patients with a primary endpoint of time to first relapse. As such, the trial was based on observing relapse events.

The sample size and power calculation assumptions for this time to first event trial were as follows:
Log-rank test for comparison of eculizumab to placebo
2:1 randomization (eculizumab:placebo)
Power 90%
Two-sided 5% level of significance
Drop-out rate 10%
Accrual period of approximately 21 months
   Relapse-free rate of 80% for the eculizumab arm at 12 months
   Hazard ratio of 0.24 (−log hazard ratio=1.41), which corresponds to a relapse-free rate of 40% for the placebo arm at 12 months

The total number of relapse(s) to be observed for this trial was 24 relapses in 24 distinct patients. With these assumptions, a maximum sample size of approximately 132 patients (88 eculizumab and 44 placebo) provides 90% power to detect a treatment difference in time to first relapse.

### 7.3. Analyses Sets

### 7.3.1. Full Analysis Set (FAS)

The population on which primary, secondary, tertiary, and other efficacy analyses were performed consisted of all patients who were randomized to treatment and who had received at least one dose of study drug (eculizumab or placebo treatment). Patients were compared for efficacy according to the treatment they were randomized to receive, irrespective of the treatment they actually received.

Time to first adjudicated relapse in the overall population was analyzed using a stratified log-rank test; a hazard ratio (HR) was estimated using a stratified Cox proportional hazards model with treatment group as covariate (prespecified analyses). Time to first adjudicated relapse in each subgroup was analyzed using an unstratified log-rank test (post hoc analyses). Adjudicated ARR was analyzed using a Poisson regression analysis (covariates: treatment group, historical ARR, and randomization strata). Other secondary endpoints were analyzed using a nonparametric analysis of covariance adjusted for baseline score and stratified by randomization strata.

### 7.3.2. Per-Protocol (PP) Set

The Per-Protocol (PP) Set is a subset of the Full Analysis Set, excluding patients with major protocol deviations. The PP population included all patients who:
had no major protocol deviations or inclusion/exclusion criteria deviations that might have potentially affected efficacy
took at least 80% of the required treatment doses while they were in the double-blind study period (for patients who had relapses any dosing after the relapse was not included in this calculation)

The PP population was fully described in the statistical analysis plan, and patients identified prior to database lock.

The reasons for exclusion comprised:
violation of inclusion/exclusion criteria
   steroid doses >20 mg/day of prednisone, or equivalent after screening (one patient in each treatment group)
   use of rituximab during the 3 months before screening (one patient receiving eculizumab)
change in supportive IST (four patients receiving eculizumab and one receiving placebo)
emergency unblinding (one patient in each treatment group)
treatment compliance of <80% (one patient receiving eculizumab).

### 7.4. Efficacy Analyses

Analyses were produced for the double-blind Study Period to compare the eculizumab group with placebo group. The analyses included efficacy, safety and PK/PD analyses.

### 7.4.1. Primary Efficacy Endpoint

The primary efficacy endpoint was time to first Adjudicated On-Trial Relapse (the original primary efficacy end point—time to first physician-determined relapse—was adopted as a sensitivity analysis). The trial met its primary efficacy objective, demonstrating a statistically significant difference between the eculizumab treatment group and the placebo group (p<0.0001). The comparison of the treatment groups for the primary endpoint used a log-rank test including the randomization stratification variables. For all reported analyses, patient populations were N=47 for placebo and N=96 for eculizumab, except as specified. These results are summarized in Table 14 and further described below. Information of each adjudicated relapse event is summarized in Table 15.

Primary Efficacy End Point: Sensitivity Analyses

The primary end point analysis was based on a stratified log-rank test of the time to first Adjudicated On-Trial Relapse using data from the full analysis set; stratification comprised low EDSS score (≤2.0), high EDSS score (≥2.5 to ≤7.0) with treatment-naïve status, high EDSS score with use of IST unchanged since last relapse, and high EDSS score with use of IST changed since last relapse. Sensitivity analyses comprised the following.

1. Adjudicated On-Trial Relapses.
a. Stratified log-rank test using data from the per-protocol set.
b. Unstratified log-rank test using data from the full analysis set.
c. Multivariate Cox proportional hazards model (with terms for treatment, baseline EDSS score dichotomized at median, historical annualized relapse rate, and IST strata at baseline) using data from the full analysis set.

2. On-Trial Relapses as determined by the treating physician.
a. Stratified log-rank test using data from the full analysis set.
b. Stratified log-rank test using data from the per-protocol set.
c. Unstratified log-rank test using data from the full analysis set.
d. Multivariate Cox proportional hazards model (with terms for treatment, baseline EDSS score dichotomized at median, historical annualized relapse rate, and IST strata at baseline) using data from the full analysis set.

3. Adjudicated On-Trial Relapses or adjudicated cases of interest with stratified log-rank test using data from the full analysis set.

TABLE 14
Summary of Primary Efficacy Endpoint Data

| | Adjudicated On-trial Relapse | | On-trial Relapses | |
|---|---|---|---|---|
| Variable/Statistic | Placebo | Eculizumab | Placebo | Eculizumab |
| Treatment Group | (N = 47) | (N = 96) | (N = 47) | (N = 96) |
| Patients with a Relapse | 20 (42.6) | 3 (3.1) | 29 (61.7) | 14 (14.6) |
| n (%) | | | | |

Estimated proportion of patients relapse-free at 95% CI

| | | | | |
|---|---|---|---|---|
| 24 weeks | 0.740 | 0.979 | 0.616 | 0.893 |
| | (0.587, 0.843) | (0.918, 0.995) | (0.461, 0.738) | (0.811, 0.941) |
| 48 weeks | 0.632 | 0.979 | 0.506 | 0.893 |
| | (0.468, 0.758) | (0.918, 0.995) | (0.355, 0.638) | (0.811, 0.941) |
| 96 weeks | 0.519 | 0.964 | 0.358 | 0.846 |
| | (0.341, 0.670) | (0.891, 0.988) | (0.213, 0.505) | (0.746, 0.909) |
| 144 weeks | 0.454 | 0.964 | 0.313 | 0.825 |
| | (0.262, 0.628) | (0.891, 0.988) | (0.169, 0.469) | (0.717, 0.895) |
| Treatment Effect (p-value) | | <0.0001 | | <0.0001 |
| Hazard Ratio (95% CI) | | 0.058 | | 0.180 |
| | | (0.017, 0,197) | | (0.095, 0.343) |
| % Risk Reduction (95% CI) | | 94.2 | | 82.0 |
| | | (80.3, 98.3) | | (65.7, 90.5) |

TABLE 15
Overview of the Adjudicated Relapses.

| | | | | | | | | | |
|---|---|---|---|---|---|---|---|---|---|
| | Age | | Historical | EDSS | | Day of | | | Severity |
| Sex | (yrs)* | Race | ARR* | score* | Supportive IST | Onset^{†} | Relapse Criteria | Relapse Type | (OSIS) |

Eculizumab group

| | | | | | | | | | |
|---|---|---|---|---|---|---|---|---|---|
| Female | 53 | White | 3.96 | 6.0 | Azathioprine + | 18 | Worsening of existing | Myelitis | Minor |
| | | | | | steroids | | symptoms | | |
| Female | 61 | Asian | 0.96 | 4.0 | Azathioprine + | 98 | Worsening of existing | Optic neuritis (right eye) | Minor |
| | | | | | steroids | | symptoms | | |
| Female | 62 | White | 1.44 | 5.0 | Mycophenolate | 338 | New onset of neurologic | Optic neuritis (both eyes) | Major |
| | | | | | mofetil | | symptoms | | |

Placebo group

| | | | | | | | | | |
|---|---|---|---|---|---|---|---|---|---|
| Male | 28 | White | 2.40 | 4.0 | Steroids | 13 | Worsening of existing | Myelitis | Major |
| | | | | | | | symptoms | | |
| Female | 39 | White | 1.44 | 3.0 | None | 21 | Worsening of existing | Myelitis | Minor |
| | | | | | | | symptoms | | |
| Female | 43 | White | 3.90 | 3.5 | None | 31 | Worsening of existing | Myelitis | Major |
| | | | | | | | symptoms | | |
| Female | 31 | White | 2.88 | 4.0 | None | 53 | Both worsening of existing | Myelitis | Major |
| | | | | | | | symptoms and symptoms in | | |
| | | | | | | | a new anatomical location | | |
| Male | 56 | Asian | 2.38 | 6.0 | Steroids | 54 | Worsening of existing | Myelitis | Major |
| | | | | | | | symptoms | | |
| Female | 51 | White | 1.92 | 4.0 | Azathioprine | 66 | Worsening of existing | Myelitis | Major |
| | | | | | | | symptoms | | |
| Female | 49 | Asian | 1.44 | 6.0 | Azathioprine + | 72 | Both worsening of existing | Myelitis | Major |
| | | | | | steroids | | symptoms and symptoms in | | |
| | | | | | | | a new anatomical location | | |
| Female | 50 | White | 2.50 | 3.0 | Azathioprine | 74 | New onset of neurologic | Myelitis | Major |
| | | | | | | | symptoms | | |
| Female | 37 | White | 1.92 | 4.5 | None | 76 | New onset of neurologic | Myelitis | Minor |
| | | | | | | | symptoms | | |
| Male | 40 | White | 1.50 | 1.5 | Azathioprine + | 88 | New onset of neurologic | Myelitis | Minor |
| | | | | | steroids | | symptoms | | |
| Female | 38 | Asian | 0.96 | 3.5 | None | 127 | New onset of neurologic | Myelitis | Minor |
| | | | | | | | symptoms | | |
| Male | 34 | Asian | 3.45 | 2.0 | Steroids | 166 | New onset of neurologic | Area postrema | Not |
| | | | | | | | symptoms | | applicable |
| Female | 23 | Black/ | 0.96 | 2.0 | Mycophenolate | 229 | Both worsening of existing | Myelitis | Major |
| | | African | | | mofetil + steroids | | symptoms and symptoms in | | |
| | | American | | | | | a new anatomical location | | |
| Female | 62 | White | 1.92 | 5.0 | Other IST + steroids | 231 | Worsening of existing | Optic neuritis (left eye) | Major |
| | | | | | | | symptoms | | |
| Female | 64 | White | 3.36 | 5.5 | Mycophenolate | 250 | New onset of neurologic | Myelitis | Major |
| | | | | | mofetil | | symptoms | | |
| Female | 41 | White | 4.76 | 4.0 | Steroids | 252 | New onset of neurologic | Optic neuritis (left eye) | Minor |
| | | | | | | | symptoms | | |
| Female | 44 | Asian | 6.38 | 3.5 | Mycophenolate | 340 | New onset of neurologic | Myelitis | Major |
| | | | | | mofetil | | symptoms | | |
| Female | 44 | White | 1.44 | 4.5 | Azathioprine | 467 | New onset of neurologic | Myelitis | Minor |
| | | | | | | | symptoms | | |
| Female | 33 | Black/ | 1.44 | 4.0 | None | 512 | New onset of neurologic | Myelitis | Major |
| | | African | | | | 558^{‡} | symptoms | Myelitis | Minor |
| | | American | | | | | New onset of neurologic | | |
| | | | | | | | symptoms | | |
| Female | 31 | Asian | 1.92 | 3.5 | None | 722 | New onset of neurologic | Myelitis | Major |
| | | | | | | | symptoms | | |

Baseline characteristic (relapses occurring during the 24 months before screening).
^{†}Relative to randomization (day 1).
^{‡}Second Adjudicated On-Trial Relapse did not contribute to the primary end point analysis.
ARR denotes annualized relapse rate, EDSS Expanded Disability Status Scale, IST immunosuppressive therapy, and OSIS Optic-Spinal Impairment Scale.

Significant improvement was observed in the eculizumab treated group in time to first relapse when compared to the placebo treated group. The number of patients with a relapse (n, %) as determined by Adjudicated On-Trial Relapse for the eculizumab treatment group was lower (3, 3.1%) when compared to the placebo treatment group (20, 42.6%). The number of patients with a relapse (n, %) as determined by the treating physician was lower for the eculizumab treatment group (14, 14.6%) when compared to the placebo treatment group (29, 61.7%).

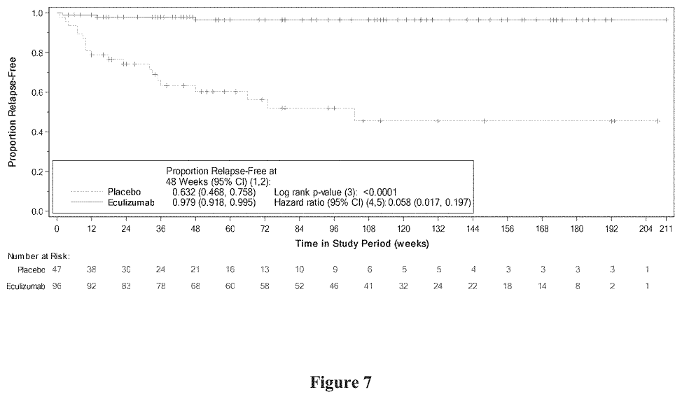

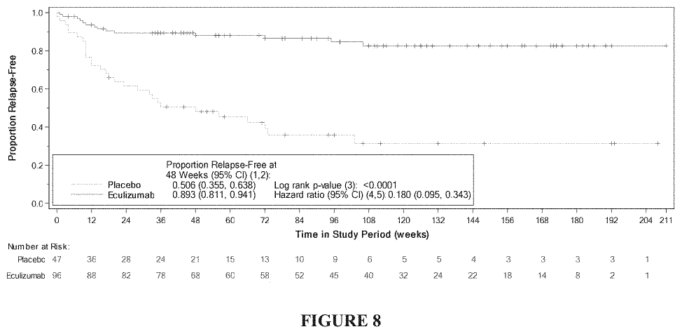

The proportion of patients relapse free determined by Adjudicated On-Trial Relapse (95% CI) was significantly greater (Log rank p-value <0.0001) for the eculizumab treatment group was 0.979 (0.918, 0.995) compared to the placebo treatment group of 0.632 (0.468, 0.758) at 48 weeks and is visually represented by Kaplan-Meier Survival Estimate in FIG. 7. The estimated proportion of patients relapse free determined by the treating Physician, (95% CI) was significantly greater (Log rank p-value <0.0001) for the eculizumab treatment group was 0.893 (0.811, 0.941) compared to the placebo treated group of 0.506 (0.355, 0.638) at 48 weeks and is visually represented by Kaplan-Meier Survival Estimate in FIG. 8.

The estimated proportion of patients relapse-free (determined by Adjudicated On-Trial

Relapse, 95% CI) was greater in the eculizumab treatment group was 0.979, with 95% CI (0.918, 0.995) when compared to the placebo treatment group of 0.740 (0.587, 0.483) at 24 weeks. The estimated proportion of patients relapse-free (determined by treating physician, 95% CI) was greater in the eculizumab treatment group of 0.893 (0.811, 0.941) when compared to the placebo treatment group of 0.616, (0.461, 0.738) at 24 weeks.

The estimated proportion of patients relapse-free determined by Adjudicated On-Trial Relapse, (95% CI) was greater in the eculizumab treatment group with 0.964 (0.891, 0.988) when compared to the placebo treatment group with 0.519 (0.341, 0.670) at 96 weeks. The estimated proportion of patients relapse-free determined by treating physician, (95% CI) was greater in the eculizumab treatment group with 0.846 (0.746, 0.909) when compared to the placebo treatment group of 0.358 (0.213, 0.505) at 96 weeks.

The estimated proportion of patients relapse-free determined by Adjudicated On-Trial Relapse, (95% CI) was greater in the eculizumab treatment group with 0.964 (0.891, 0.988) when compared to the placebo treatment group of 0.454 (0.262, 0.628) at 144 weeks. The estimated proportion of patients relapse-free determined by treating physician, (95% CI) was greater in the eculizumab treatment group with 0.825 (0.717, 0.895) when compared to the placebo treatment group of 0.313 (0.169, 0.469) at 144 weeks (Table 20).

The treatment effect (p-value) for the eculizumab treatment group when compared to placebo treatment was (p<0.0001) for both time to Adjudicated On-Trial Relapse as well as time to relapse as determined by treating physician. The hazard ratio for those treated with eculizumab (time to first Adjudicated On-Trial Relapse) was 0.058 (0.017, 0.197); a 94.2% (80.3%, 98.3%) reduction in the risk of relapse was observed in the eculizumab treatment group compared to the placebo treatment group (Table 20). A hazard ratio in time to first relapse as determined by treating physician of 0.180 (0.095, 0.343) and an 82.0% (65.7%, 90.5%) reduction in the risk of relapse was observed in the eculizumab treatment group (Table 20).

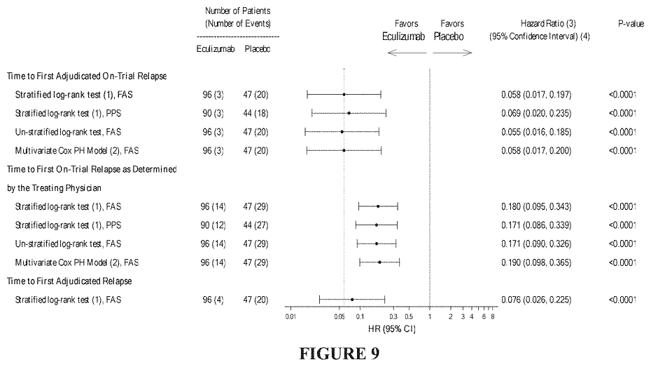

Results of a sensitivity comparison of the primary endpoint, using a Cox proportional hazards regression model with treatment group indicator, randomization stratification variables, and historical annualized relapse rate as the only covariates in the model are visually represented in FIG. 9.

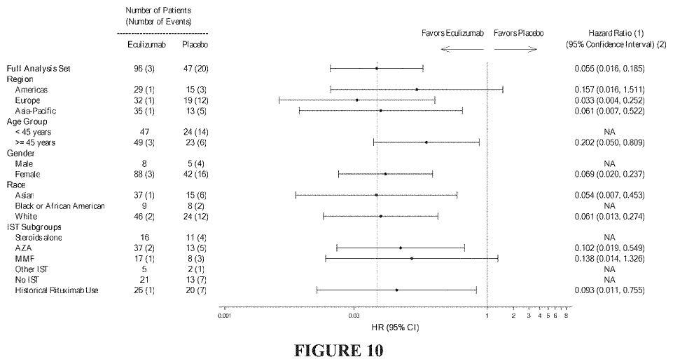

Results of a second sensitivity comparison of the primary endpoint used a Cox proportional hazards regression model with treatment group indicator, randomization stratification variables, demographics, and region as the only covariates in the model. Region was defined based on the sites for the trial and include North America and South America pooled as the Americas, Europe, and Asia-Pacific are visually represented in FIG. 10 (as determined by Adjudicated On-Trial Relapse) and FIG. 11 (as determined by treating physician).

### 7.4.2. Secondary Efficacy Analysis

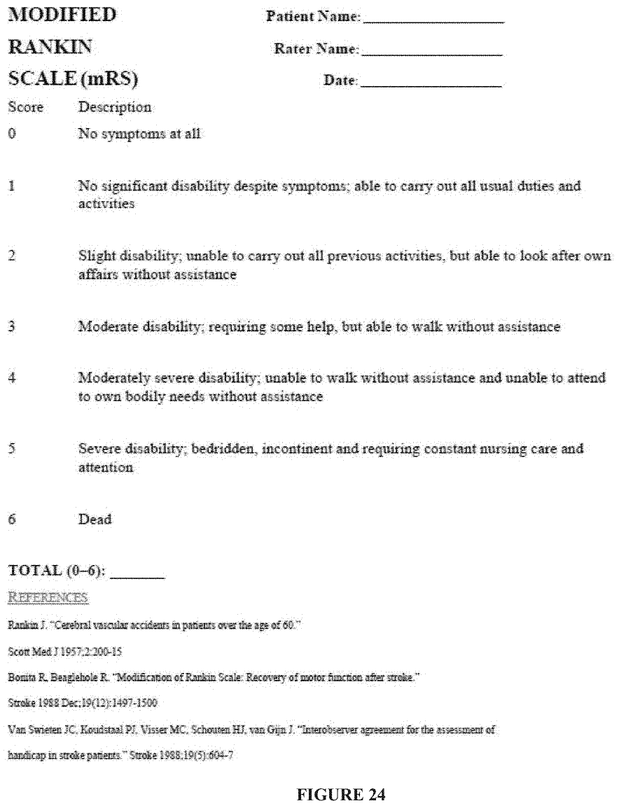

Hierarchically ordered secondary efficacy endpoints were: adjudicated annualized relapse rate (ARR), followed by changes in measures of disability and neurologic function (EDSS, modified Rankin Scale [mRS] (FIG. 24), and Hauser Ambulation Index [HAI]), and quality of life (European Quality of Life 5-dimension [EQ-5D-3L] questionnaire).

During the Study Period, baseline was defined as the last available assessment prior to treatment for all patients regardless of their treatment group.

Unless otherwise specified, the secondary efficacy analyses used the available data from the Study Period. Hypothesis testing comparing eculizumab treatment with placebo treatment for the secondary efficacy analyses were performed using a closed testing procedure with the following rank order:

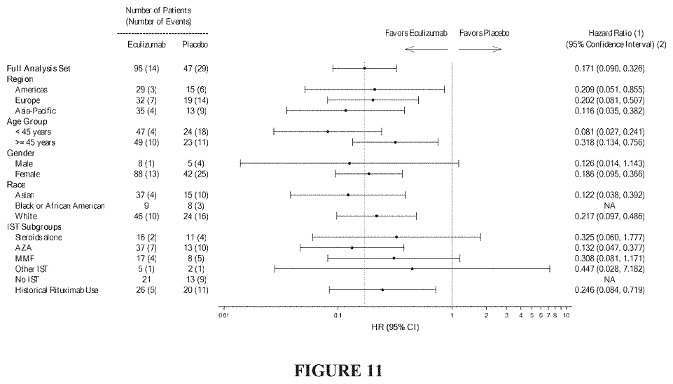

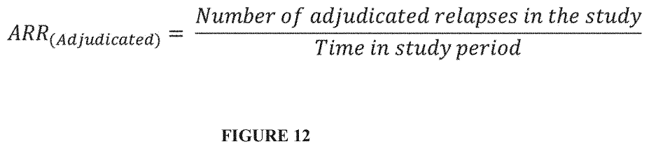

1. Adjudicated On-Trial annualized relapse rate (ARR) (FIG. 12)

2. Change from baseline in Expanded Disability Status Scale EDSS score at the EOS

3. Change from baseline in modified Rankin Scale (mRS) at the EOS

4. Change from baseline in Hauser Ambulation Index (HAI) at the EOS

5. Change from baseline in EuroQoL Visual Analogue Scale (EQ-5D VAS) at the EOS

6. Change in EQ-5D Index at the EOS

The hypothesis testing proceeded from highest rank (#1) Adjudicated On-Trial annualized relapse rate (ARR) to the lowest rank (#5) Change in EQ-5D index at the EOS, and if statistical significance was not achieved at an endpoint (p≤0.05), then endpoints of lower rank were not considered to be statistically significant. Confidence intervals and p-values were presented for all secondary efficacy endpoints are for descriptive purposes, regardless of the outcome of the closed testing procedure. The EQ-5D has two endpoints, the index score and EQ-5D VAS. For the purposes of this closed testing procedure, the EQ-5D VAS score was analyzed first and the EQ-5D index is analyzed second for the EQ-5D analyses.

The comparison of the two treatment groups for the primary endpoint, adjudicated On-Trial annualized relapse rate (ARR), used Poisson regression analysis. Treatment group, the stratification variables, and baseline annualized relapse rate were covariates in the analysis, and the log of time in the trial was used as the offset variable. The results of this analysis are summarized in Table 16. As determined by Adjudicated On-Trial relapses, the eculizumab treatment group had fewer total number of relapses (3) when compared to the placebo treatment group (21) (Table 16). As determined by treating physician, the eculizumab treatment group had fewer total number of relapses (14) compared to the placebo treatment group (31). A greater number of total Patient-years was evaluated for the eculizumab treatment group (171.32) compared to the placebo treatment group (52.41), indifferent to relapse determination mechanism. A lower Unadjusted ARR (95% CI) was observed for the eculizumab treatment group by Adjudicated On-Trial Relapse of 0.018 (0.006, 0.054) when compared to the placebo treatment group of 0.401 (0.261, 0.615). A significantly lower adjusted ARR (95% CI, p<0.0001) was observed in the eculizumab treatment group by Adjudicated On-Trial Relapse with 0.016 (0.005, 0.050) as compared to the placebo treatment group with 0.350; (0.199, 0.616) (Table 16).

A lower Unadjusted ARR (95% CI) was observed for the eculizumab treatment group by On-Trial Relapse determined by treating physician with 0.082 (0.048, 0.138) when compared to the placebo treatment group with 0.592 (0.416, 0.841). A significantly lower adjusted ARR (95% CI, p<0.0001) was observed in the eculizumab treatment group by On-Trial Relapse determined by treating physician of 0.066 (0.036, 0.120) as compared to the placebo treatment group with 0.446 (0.272, 0.732) (Table 16). For Adjudicated On-Trial Relapses, the treatment effect rate ratio was 0.045 (Adjusted ARR Eculizumab/Placebo). For relapses determined by treating physician the treatment effect rate ratio was 0.147 (Adjusted ARR Eculizumab/Placebo).

Further prespecified (sensitivity) analyses were conducted for EDSS, mRS, HAI, EQ-5D-3L VAS, and EQ-5D-3L index scores for changes from baseline to each scheduled visit during the first year using a mixed model for repeated measures with terms for treatment, visit, treatment-by-visit interaction, baseline score (for each scale), and randomization strata.

Missing data relating to secondary efficacy analyses of changes from baseline to end of study (excluding sensitivity analyses) were replaced as explained below.
The last observed scores from protocol-scheduled visits were used to replace missing data from assessments undertaken 6 weeks after the first relapse and also missing end-of-study scores for patients without a relapse.
Baseline values were used for patients with no post-baseline assessments.
The last scores before a second relapse were used for patients experiencing second relapses during the 6-week recovery phase following the first relapse.

Missing data were not imputed for any other analysis.

TABLE 16
Summary of Annualized Relapse Rate in Treatment Arms

| | Adjudicated On-trial Relapse | | On-trial Relapses | |
|---|---|---|---|---|
| Analysis Description | Placebo | Eculizumab | Placebo | Eculizumab |
| Treatment Group | (N = 47) | (N = 96) | (N = 47) | (N = 96) |
| Total number of relapses | 21 | 3 | 31 | 14 |
| Total Patient-years | 52.41 | 171.32 | 52.41 | 171.32 |
| Unadjusted ARR | 0.401 | 0.018 | 0.592 | 0.082 |
| (95% CI) | (0.261, 0.615) | (0.006, 0.054) | (0.416, 0.841) | (0.048, 0.138) |
| Adjusted ARR | 0.350 | 0.016 | 0.446 | 0.066 |
| (95% CI) | (0.199, 0.616) | (0.005, 0.050) | (0.272, 0.732) | (0.036, 0.120) |
| Treatment effect: Rate | | 0.045 | | 0.147 |
| ratio (Ecu/Placebo) | | | | |
| 95% CI | | (0.013, 0.151) | | (0.078, 0.278) |
| p-value | | <0.0001 | | <0.0001 |

Based an a Poisson regression adjusted for randomization strata and historical [symbol] in 24 months prior to screening.

Results of additional secondary efficacy endpoints are summarized in Table 17 and Table 18 below.

TABLE 17
Secondary Efficacy End Points

| | Adjusted Rates (95% CI) | | Rate Ratio (95% CI) for | |
|---|---|---|---|---|
| | Eculizumab (N = 96) | Placebo (N = 47) | Eculizumab vs. Placebo | P value |
| Adjudicated annualized relapse rate | 0.016 (0.005-0.050) | 0.350 (0.199-0.616) | 0.045 (0.013-0.151) | <0.001 |

Adjudicated annualized relapse rates (A) is shown for the full analysis set.
Hypothesis testing comparing eculizumab treatment with placebo for the secondary efficacy analyses were performed using a closed testing procedure with the hierarchy as presented.
CI denotes confidence interval.

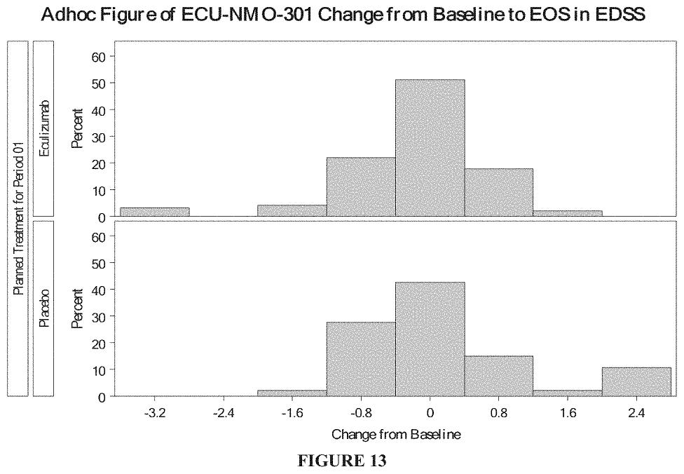

The primary analysis for the change from baseline in EDSS score to the EOS visit (i.e., 6-week post-relapse for the patients who had relapses or end of treatment period visit for patients who do not have relapses) was a Nonparametric ANCOVA with treatment group, baseline EDSS, and IST status at randomization as covariates, and results are visually depicted in (FIG. 13). If a patient experienced a second relapse during the 6-week recovery phase after the initial relapse the last EDSS score prior to the second attack was used for the analysis. If a patient had no follow-up assessments, a change from baseline of 0 (i.e., baseline value carried forward) was used.

At baseline, median (range) EDSS (4.0 [1.0-7.0]) and HAI (2 [0-8]) scores were similarly distributed between treatment groups and indicated moderate-to-severe disability. The placebo treatment group was observed to have a mean (+/−SD) change in EDSS score from baseline to EOS of 0.12 (+/−0.945, min=−2.0, max=2.5), while the eculizumab treatment group was observed to have a mean change in EDSS score from baseline to EOS of −0.18 (+/−0.814, min=−3.5, max=1.5; p=0.0597). Regarding changes from baseline in EDSS scores, among eculizumab- and placebo-treated patients, respectively, 51.0% and 42.6% had no change, 29.2% and 29.8% had ≥0.5-point improvement, and 19.8% and 27.7% had ≥0.5-point worsening. Changes from baseline in EDSS were small and tended to favor eculizumab over placebo. The modest effect observed on the accumulation of disability may have been because a large proportion of patients did not experience a relapse, and those that did were not followed beyond 6 weeks after the relapse. As the P value for the next secondary endpoint (change in EDSS score) exceeded 0.05, P values for the others were considered nonconfirmatory. The median change for both treatment groups was 0.0.

Overall, results for EDSS, MRS, HAI, and EQ-5D-3L showed no worsening of disability or health-related quality of life in either group and the distribution of changes favored eculizumab over placebo.

The sensitivity analysis for the change from baseline in EDSS score to the EOS period visit (i.e., 6-week post-relapse for the patients who have relapses or EOS visit for patients who do not have relapses) was ANCOVA with treatment group, baseline EDSS, and IST status at randomization as covariates in the subset of the FAS population who do have a follow-up assessment (i.e., FAS patients without a follow-up assessment were excluded from the analysis). If a patient experienced a second relapse during the 6-week recovery phase after the initial relapse the last EDSS score prior to the second relapse was used for the analysis. The least squares (LS) mean (+/−SEM) change from baseline of the eculizumab treatment group was −0.26 (+/−0.096, min=−0.45, max=−0.07). The LS mean change from baseline of the placebo treatment group was 0.03 (+/−0.133; min=−0.23, max=0.29), and the difference in LS mean between these two groups was −0.29 (+/−0.152, min=−0.59, max=0.01, p=0.0603). Results from all sensitivity analyses are summarized in Table 19 below.

TABLE 19
Summary of Secondary Efficacy Endpoint Sensitivity Analysis Results

| Change from Baseline | | | | Difference in LS |
|---|---|---|---|---|
| to End of Study (EOS) | | Placebo | Eculizumab | Mean (95% CI) |
| Variable | Statistic | (N = 47) | (N = 96) | p-value |
| EDSS Score | LS Mean (SEM) | 0.03 (0.133) | −0.26 (0.096) | −0.29 (0.152) |
| | 95% CI | (−0.23, 0.29) | (−0.45, −0.07) | (−0.59, 0.01) |
| | | | | 0.0603 |
| mRS | LS Mean (SEM) | 0.00 (0.115) | −0.32 (0.084) | −0.032 (0.129) |
| | 95% CI | (−0.23, 0.23) | (−0.48, −0.15) | (−0.57, −0.06) |
| | | | | 0.0154 |
| HAI | LS Mean (SEM) | 0.37 (0.201) | −0.50 (0.147) | −0.87 (0.227) |
| | 95% CI | (−0.03, 0.77) | (−0.79, −0.21) | (−1.32, −0.42) |
| | | | | 0.0002 |
| EQ-5D VAS | LS Mean (SEM) | 1.33 (2.573) | 7.76 (1.892) | 6.43 (2.935) |
| | 95% CI | (−3.75, 6.42) | (4.02, 11.50) | (0.63, 12.23) |
| | | | | 0.0302 |
| EQ-5D Index | LS Mean (SEM) | −0.03 (0.029) | 0.06 (0.021) | 0.09 (0.033) |
| | 95% CI | (−0.08, 0.03) | (0.02, 0.10) | (0.02, 0.15) |
| | | | | 0.0075 |

Note:
p-value from ANCOVA adjusted for baseline score and randomization strata.

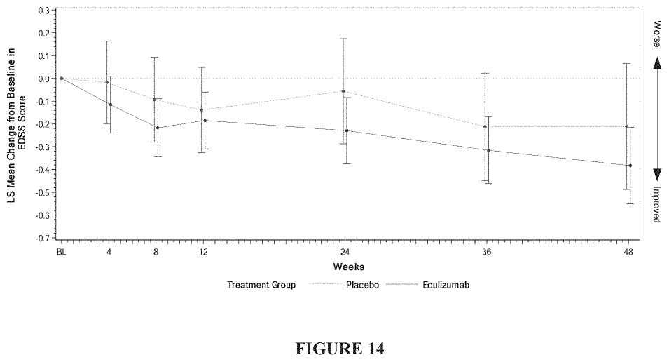

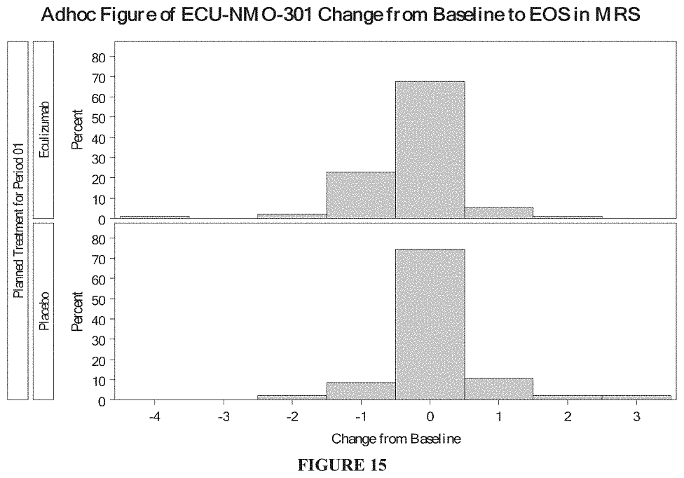

In addition, sensitivity analyses for the change from Baseline in EDSS were analyzed using a mixed model for repeated measures with baseline EDSS score, IST status at randomization, treatment group indicator, trial visit and trial visit by treatment group interaction as covariates, and the results are visually represented in FIG. 14. All post-baseline EDSS scores were included in the models; patients without any post-baseline scores were not included. The primary analysis for the change from baseline in mRS score to the end of the study period visit (i.e., 6-week post-relapse for the patients who have relapses or EOS visit for patients who do not have relapses) was a nonparametric ANCOVA with treatment group, baseline mRS, EDSS strata at randomization, and IST status at randomization as covariates, and is visually represented in FIG. 15. The result of sensitivity analysis, including that of physician-determined relapses, were consistent with the primary analysis.

Of the 45 physician-determined relapses, 24 (53.3%) were adjudicated positively. If a patient experienced a second relapse during the 6-week recovery phase after the initial relapse, the last mRS score prior to the second attack was used for the analysis. If a patient has no follow-up assessments, a change from baseline of 0 (i.e., baseline value carried forward) was used. The mean change (+/−SD) from baseline to EOS mRS of the treatment group given eculizumab was −0.2 (+/−0.72, min=−4, max=2). The mean change from baseline to EOS mRS of the treatment group given placebo was 0.1 (+/−0.75, min=−2, max=3; p=0.0154). The median change for both treatment groups was 0.0 (Table 18).

A sensitivity analysis for the change from baseline in mRS score to the end of the Study Period Visit was ANCOVA with treatment group, baseline mRS, EDSS strata at randomization, and IST status at randomization as covariates in the subset of the FAS population who do have a follow-up assessment (i.e., FAS patients without a follow-up assessment will be excluded from the analysis). The least squares (LS) mean (+/−SEM) change from baseline of the eculizumab treatment group was −0.32 (+/−0.084, min=−0.48, max=−0.15). The LS mean change from baseline of the placebo treatment group was 0.00 (+/−0.115, min=−0.23, max=0.23). The difference in LS mean between these two groups for mRS was −0.032 (+/−0.129, min=−0.57, max=−0.06, p=0.0154) (Table 16).

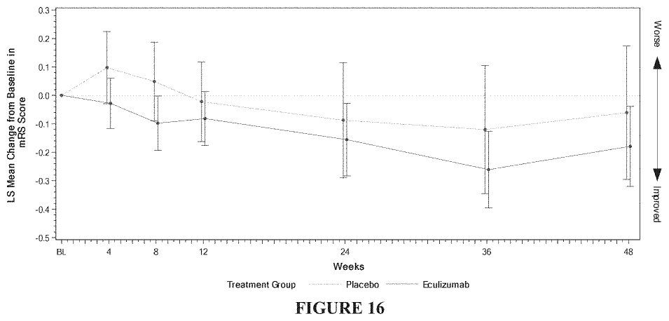

In addition, sensitivity analyses for the change from Baseline in mRS were analyzed using a mixed model for repeated measures with baseline mRS, IST status at randomization, treatment group indicator, trial visit and trial visit by treatment group interaction as covariates. All post-baseline mRS scores were included in the models; patients without any post-baseline scores were not included Results are visually depicted in (FIG. 16).

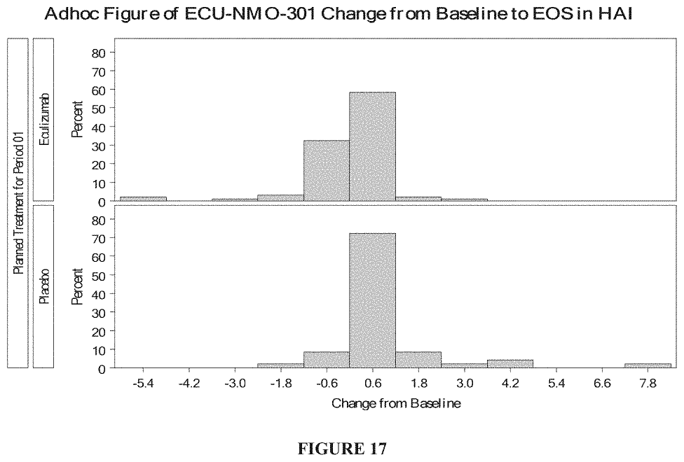

Changes from baseline in the HAI to the EOS period visit (i.e., 6-week post-relapse for the patients who have relapses or EOS visit for patients who do not have relapses) was a Nonparametric ANCOVA with treatment group, baseline HAI, and IST status at randomization as covariates in the subset of the FAS population who do have a follow-up assessment (i.e., FAS patients without a follow-up assessment were excluded from the analysis). Results are visually depicted in (FIG. 17). The mean (+/−SD) change from baseline in the eculizumab treatment group was −0.4 (+/−1.08, min=−5, max=3), with a median change of 0.0. The mean (+/−SD) change from baseline in the placebo treatment group was 0.5 (+/−1.61, min=−2, max=8), with a median change of 0.0, (p=0.0002).

A sensitivity analysis for the change from baseline in HAI score to the end of the Study Period Visit was ANCOVA with treatment group, baseline HAI, EDSS strata at randomization, and IST status at randomization as covariates in the subset of the FAS population who do have a follow-up assessment (i.e., FAS patients without a follow-up assessment will be excluded from the analysis). The least squares (LS) mean (+/−SEM) change from baseline of the eculizumab treatment group was −0.50 (+/−0.147, min=−0.79, max=−0.21). The LS mean change from baseline of the placebo treatment group was 0.37 (+/−0.201, min=−0.03, max=0.77). The difference in LS mean between these two groups for HAI was −0.87 (+/−0.227, min=−1.32, max=−0.42, p=0.0002) (Table 16).

In addition, sensitivity analyses for the change from Baseline in HAI were analyzed using a mixed model for repeated measures with baseline HAI, IST status at randomization, treatment group indicator, trial visit and trial visit by treatment group interaction as covariates. All post-baseline HAI scores were included in the models; patients without any post-baseline scores were not included. Results are visually depicted in FIG. 18.

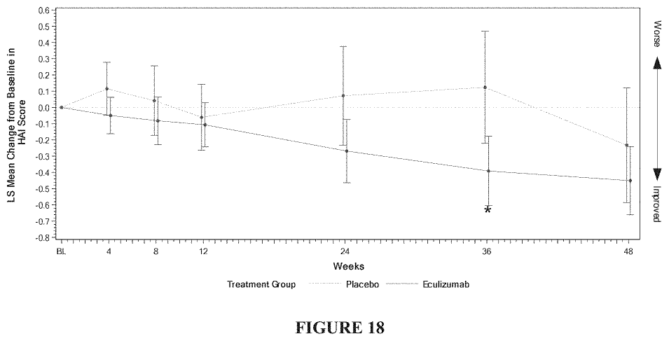

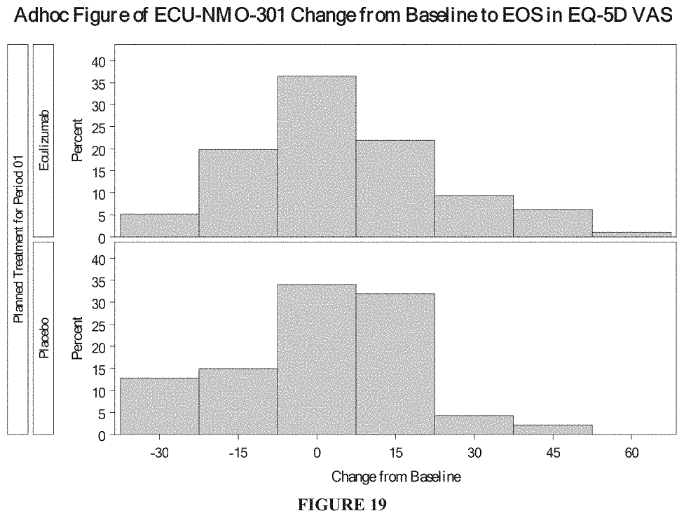

Changes from baseline in the EQ-5D VAS to the EOS period visit (i.e., 6-week post-relapse for the patients who have relapses or EOS visit for patients who do not have relapses) was a nonparametric ANCOVA with treatment group, baseline EQ-5D VAS, and IST status at randomization as covariates in the subset of the FAS population who do have a follow-up assessment (i.e., FAS patients without a follow-up assessment were excluded from the analysis). Results are visually depicted in FIG. 19. The mean (+/−SD) change from baseline in the eculizumab treatment group was 5.4 (+/−18.53, min=−30, max=60), with a median change of 0.0. The mean (+/−SD) change from baseline in the placebo treatment group was 0.6 (+/−16.39 min=−28, max=40), with a median change of 0.0, (p=0.0309).

A sensitivity analysis for the change from baseline in EQ-5D VAS score to the end of the Study Period Visit was ANCOVA with treatment group, baseline EQ-5D VAS, EDSS strata at randomization, and IST status at randomization as covariates in the subset of the FAS population who do have a follow-up assessment (i.e., FAS patients without a follow-up assessment will be excluded from the analysis). The least squares (LS) mean (+/−SEM) change from baseline of the eculizumab treatment group was 7.76 (+/−1.892, min=4.02, max=11.50). The LS mean change from baseline of the placebo treatment group was 1.33 (+/−2.573, min=−3.75, max=6.42). The difference in LS mean between these two groups for EQ-5D VAS was 6.43 (+/−2.935, min=0.63, max=12.23, p=0.0302).

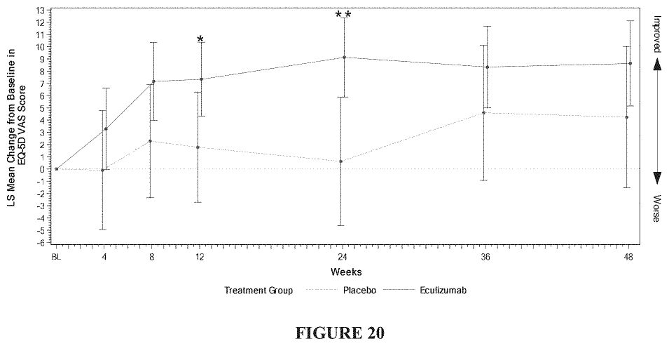

In addition, sensitivity analyses for the change from Baseline in EQ-5D VAS were analyzed using a mixed model for repeated measures with baseline EQ-5D VAS, IST status at randomization, treatment group indicator, trial visit and trial visit by treatment group interaction as covariates. All post-baseline EQ-5D VAS scores were included in the models; patients without any post-baseline scores were not included. Results are visually depicted in FIG. 20.

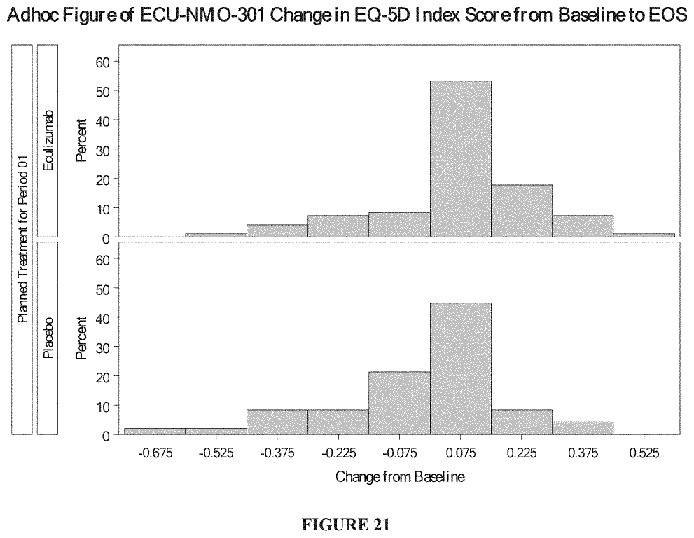

Changes from baseline in the EQ-5D Index Score to the EOS period visit (i.e., 6-week post-relapse for the patients who have relapses or EOS visit for patients who do not have relapses) was a nonparametric ANCOVA with treatment group, baseline EQ-5D Index Score, and IST status at randomization as covariates in the subset of the FAS population who do have a follow-up assessment (i.e., FAS patients without a follow-up assessment were excluded from the analysis). Results are visually depicted in FIG. 21. The mean (+/−SD) change from baseline in the eculizumab treatment group was 0.05 (+/−0.179, min=−0.57, max=0.56), with a median change of 0.03. The mean (+/−SD) change from baseline in the placebo treatment group was −0.04 (+/−0.212, min=−0.67, max=0.41), with a median change of 0.0, (p=0.0077).

A sensitivity analysis for the change from baseline in EQ-5D Index Score to the end of the Study Period Visit was ANCOVA with treatment group, baseline EQ-5D Index score, EDSS strata at randomization, and IST status at randomization as covariates in the subset of the FAS population who do have a follow-up assessment (i.e., FAS patients without a follow-up assessment will be excluded from the analysis). The least squares (LS) mean (+/−SEM) change from baseline of the eculizumab treatment group was 0.06 (+/−0.021, min=0.02, max=0.10). The LS mean change from baseline of the placebo treatment group was −0.03 (+/−0.029, min=−0.08, max=0.03). The difference in LS mean between these two groups for EQ-5D Index Score was 0.09 (+/−0.033, min=0.02, max=0.15, p=0.0075).

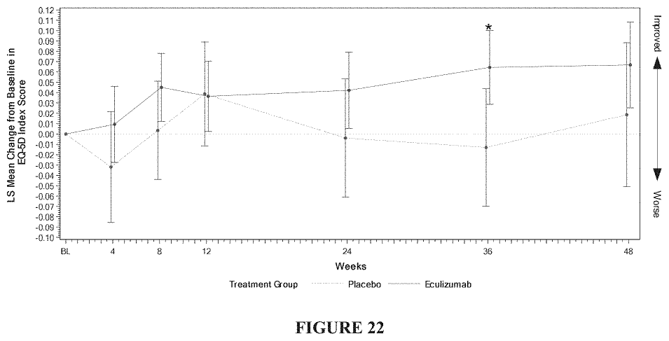

In addition, sensitivity analyses for the change from Baseline in EQ-5D Index Score were analyzed using a mixed model for repeated measures with baseline EQ-5D Index Score, IST status at randomization, treatment group indicator, trial visit and trial visit by treatment group interaction as covariates. All post-baseline EQ-5D Index scores were included in the models; patients without any post-baseline scores were not included. Results are visually depicted in FIG. 22.

### 7.4.3 Additional Relapse Data

Overall, 24% of patients with identified NMOSD did not receive ISTs during the trial. The outcome measure was the adjudicated relapse rate (ARR) during the trial as determined by an independent panel vs rates during 24 months prior to the study. Baseline annualized relapse rates (ARRs; eculizumab, mean=1.94 [standard deviation=0.896]; placebo, 2.07[1.037]) between eculizumab and placebo treated groups were similar. Prespecified IST subgroups were steroids alone, azathioprine (AZA) with/without steroids, mycophenolate mofetil (MMF) with/without steroids, other IST, no IST and historic rituximab use. Other subgroups included age, race, gender and geographic region.

The ARR in the eculizumab and placebo groups, respectively, among patients receiving steroids alone (n=27) was 0/16 (0.0%) and 4/11 (36.4%), in the AZA subgroup (n=50) was 2/37 (5.4%) and 5/13 (38.5%), and in the MMF subgroup (n=25) was 1/17 (5.9%) and 3/8 (37.5%). The ARR in the eculizumab and placebo groups, respectively, among patients from Europe (n=51) was 1/32 (3.1%) and 12/19 (63.2%), from Asia Pacific (n=48) was 1/35 (2.9%) and 5/13 (38.5%), and the Americas (n=44) was 1/29 (3.4%) and 3/15 (20.0%). Similarly, a lower ARR with eculizumab vs placebo was observed in the IST, no IST and historic rituximab use subgroups, and by age, race and gender. Analyses of ARR in PREVENT showed a treatment benefit of eculizumab vs placebo across all subgroups, including IST use and geographic region.

On-Trial Relapses were assessed based on Adjudication status (positively or negatively) for both treatment arms. A summary of these results is provided in Table 20 below.

Cases of interest (potential relapses identified by treating physician) were also assessed based on adjudication status. A summary of the case outcomes can be found in Table 21 below

TABLE 21
Summary of Adjudication Status for Cases of Interest

| | | Placebo | Eculizumab | Total |
|---|---|---|---|---|
| Variable | Statistic | (N = 12) | (N = 21) | (N = 33) |
| Adjudicated positively | n (%) | 1 (8.3) | 1 (4.8) | 2 (6.1) |
| Adjudicated negatively | n (%) | 11 (91.7) | 20 (95.2) | 31 (93.9) |

On-Trial relapses were also categorized by severity. In adjudicated On-Trial relapses (Table 22), the treatment group given eculizumab had fewer overall events (3) compared to the placebo treatment group (20). The treatment group given eculizumab also had fewer major events (1) compared to the placebo treatment group (13). The treatment group given eculizumab also had fewer minor events (2) compared to the placebo treatment group (7). In all on-trial relapses (Table 23), the treatment group given eculizumab had fewer overall events (13) compared to the placebo treatment group (30). The eculizumab treatment group also had fewer major events (3) compared to the placebo treatment group (17).

TABLE 22
Adjudicated On-Trial Relapses by Severity

| | | Placebo | | | Eculizumab | | |
|---|---|---|---|---|---|---|---|
| | | (N = 21) | | | (N = 3) | | |
| | | Total | | | Total | | |
| | | Number | | | Number | | |
| Relapse | Statistic | of Events | Major | Minor | of Events | Major | Minor |
| Overall | n (%) | 20 | 13 (65.0) | 7 (35.0) | 3 | 1 (33.3) | 2 (66.7) |
| Optic Neuritis | n (%) | 2 | 1 (50.0) | 1 (50.0) | 2 | 1 (50.0) | 1 (50.0) |
| Transverse | n (%) | 18 | 12 (66.7) | 6 (33.3) | 1 | 0 (0.0) | 1 (100.0) |
| Myelitis | | | | | | | |
| Other | n | 1 | | | 0 | | |

On-Trial Relapses were categorized by type of relapse (Optic Neuritis, Myelitis, or other) as determined by Adjudicated On-Trial Relapse in Table 24 below

TABLE 24
On-Trial Relapses by Severity

| | | Placebo | | Eculizumab | |
|---|---|---|---|---|---|
| Variable | Statistic | (N = 21) | | (N = 3) | |
| Optic Neuritis | n (%) | 2 | (9.5) | 2 | (66.7) |
| Unilateral Left | n (%) | 2 | (100.0) | 0 | (0.0) |
| Unilateral Right | n (%) | 0 | (0.0) | 1 | (50.0) |
| Bilateral | n (%) | 0 | (0.0) | 1 | (50.0) |
| Myelitis | n (%) | 18 | (85.7) | 1 | (33.3) |
| Other | n (%) | 1 | (4.8) | 0 | (0.0) |

On-Trial Relapses were categorized by type of relapse (Optic Neuritis, Myelitis, or other) as determined by treating physician in Table 25 below

TABLE 25
On-Trial Relapses by Type of Relapse

| | | Placebo | | Eculizumab | |
|---|---|---|---|---|---|
| Variable | Statistic | (N = 31) | | (N = 14) | |
| Optic Neuritis | n (%) | 6 | (19.4) | 6 | (42.9) |
| Unilateral Left | n (%) | 4 | (66.7) | 4 | (66.7) |
| Unilateral Right | n (%) | 0 | (0.0) | 1 | (16.7) |
| Bilateral | n (%) | 2 | (33.3) | 1 | (16.7) |
| Traverse Myelitis | n (%) | 26 | (83.9) | 7 | (50.0) |
| Partial | n (%) | 14 | (53.8) | 4 | (57.1) |
| Complete | n (%) | 1 | (3.8) | 1 | (14.3) |
| Partial Longitudinally extensive | n (%) | 11 | (42.3) | 1 | (14.3) |
| Complete Longitudinally extensive | n (%) | 0 | (0.0) | 1 | (14.3) |
| Brainstem Symptoms | n (%) | 1 | (3.2) | 0 | (0.0) |
| Cerebral Symptoms | n (%) | 0 | (0.0) | 1 | (7.1) |
| Other | n (%) | 2 | (6.5) | 0 | (0.0) |

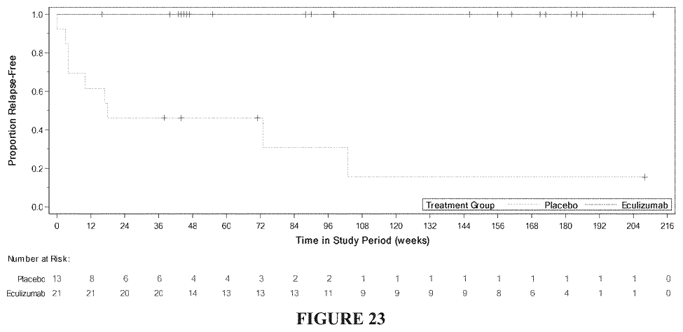

On-Trial relapses as determined by the treating physician for patients with no baseline IST use was also assessed. Kaplan-Meier Survival Estimates were generated and are visually depicted in FIG. 23.

A total of 34 patients (23.8%) did not receive ISTs during the study. Any ISTs: 3/75 patients (4%) receiving eculizumab and 13/34 patients (38%) receiving placebo experienced adjudicated relapses.

In certain cases, relapses resulted in hospitalizations. A summary of On-Trial Relapse hospitalizations is provided in Table 26 below, characterizing the number of patients requiring hospitalization, the number of relapses that resulted in hospitalization, the total number of patient-years in the study period, and the annualized relapse-related hospitalization rate.

TABLE 26
Summary of On-Trial Relapse Hospitalizations.

| | | Placebo | Eculizumab |
|---|---|---|---|
| Variable | Statistic | (N = 47) | (N = 96) |
| Number of patients with an | n (%) | 15 (31.9) | 6 (6.3) |
| On-Trial relapse requiring | | | |
| hospitalization | | | |
| Total number of On-Trial relapses | Sum | 16 | 6 |
| requiring hospitalization | | | |
| Total number of patient-years in | Sum | 52.41 | 171.32 |
| study period | | | |
| Annualized relapse-related | Rate | 0.31 | 0.04 |
| hospitalization rate | | | |
| | 95% CI | (0.19, 0.50) | (0.02, 0.08) |
| | p-value | | <0.0001 |

In certain cases, relapses resulted in hospitalization and treatment. These are summarized in Table 27 below. Total number of patients, total number of relapses, total number of patient-years and annualized hospital rate are characterized according to acute treatment type and trial treatment arm.

TABLE 27
Summary of On-Trial Relapse Hospitalizations and Treatment

| | | | |
|---|---|---|---|
| | | Placebo | Eculizumab |
| Variable | Statistic | (N = 47) | (N = 96) |

Number of patients with an
On-Trial relapse requiring
hospitalization

| | | | | | |
|---|---|---|---|---|---|
| High-dose oral steroids | n (%) | 6 | (12.8) | 7 | (7.3) |
| IV Methylprednisolone | n (%) | 22 | (46.8) | 12 | (12.5) |
| Plasma Exchange | n (%) | 9 | (19.1) | 4 | (4.2) |
| IVIg | n (%) | 0 | (0.0) | 0 | (0.0) |

| | | | |
|---|---|---|---|
| Total number of On-Trial relapses | | | |
| requiring acute treatment with | | | |
| Hight-dose oral steroids | Sum | 6 | 7 |
| IV Methylprednisolone | Sum | 22 | 12 |
| Plasma Exchange | Sum | 10 | 4 |
| Total number of patient-years in | Sum | 52.41 | 171.32 |
| study period | | | |
| Annualized relapse-related | | | |
| High-dosed oral steroid rate | Rate | 0.11 | 0.04 |
| | 95% CI | (0.05, 0.25) | (0.02, 0.09) |
| | p-value | | 0.0733 |
| IV Methylprednisolone rate | Rate | 0.42 | 0.07 |
| | 95% CI | (0.28, 0.64) | (0.04, 0.12) |
| | p-value | | <0.0001 |
| Plasma Exchange rate | Rate | 0.19 | 0.02 |
| | 95% CI | (0.10, 0.35) | (0.01, 0.06) |
| | p-value | | 0.0001 |

Relapses in Japanese patients were characterized by relapse determination (On-Trial or adjudicated) and are summarized in Table 28 below

TABLE 28
Summary of Relapses in Japanese Patients

| | Placebo | Eculizumab |
|---|---|---|
| Type Release | (N = 5) | (N = 9) |
| Adjudicated On-Trial Relapses | 2 (40%) | 1 (11%) |
| On-Trial Relapses | 3 (60%) | 2 (22%) |

### 7.4.4 Safety Analysis

To determine safety of treatment, study duration and drug compliance were analyzed in Table 29 below

An overview of key safety measures, including adverse events, serious events, deaths, withdrawals and infections are summarized in Table 30 below. Specifically, patients receiving eculizumab spent longer in the trial than those receiving placebo (total duration, 172.8 patient-years vs 53.1 patient-years, respectively; median duration, 91 weeks vs 43 weeks, respectively). As patients receiving eculizumab generally spent more time in the study than those receiving placebo, adverse-event rates (expressed per 100 patient-years) are reported alongside proportions of patients with adverse events. The adverse event reported at the highest rate in each group was headache (55.0 and 37.6 per 100 patient-years for eculizumab and placebo groups, respectively) and the rates for treatment-related adverse events were 211.8 and 163.7 per 100 patient-years, respectively.

TABLE 30
Overview of Key Safety Results

| | Placebo (N = 47) | | | Eculizumab (N = 96) | | |
|---|---|---|---|---|---|---|
| | Pt-years (PY) = 53.1 | | | Pt-years (PY) = 172.8 | | |
| | | Rate per | Patients | | Rate per | Patients |
| | Events n | 100 PY | n (%) | Events n | 100 PY | n (%) |
| Any Adverse Event (AE) | 617 | 1160.9 | 45 (95.7) | 1295 | 749.3 | 88 (91.7) |
| Any Serious AE (SAE) | 47 | 88.4 | 26 (55.3) | 53 | 30.7 | 30 (31.3) |
| Death | | | 0 (0.0) | | | 1 (1.0) |
| AEs Leading to Withdrawal | 3 | 5.6 | 2 (4.3) | 0 | 0.0 | 0 (0.0) |
| of Study Drug | | | | | | |
| SAEs Leading to Withdrawal | 2 | 3.8 | 2 (4.3) | 0 | 0.0 | 0 (0.0) |
| of Study Drug | | | | | | |
| Meningococcal infections | 0 | 0 | 0 (0.0) | 0 | 0.0 | 0 (0.0) |

Overall AE rates per 100 patient-years with NMOSD relapses included as AEs were 749 and 1161 for eculizumab and placebo, respectively, and without NMOSD relapses were 745 and 1127, respectively.

Headache and upper respiratory tract infection were reported more commonly in patients receiving eculizumab than in those receiving placebo. Two patients discontinued treatment because of AEs, both receiving placebo. One patient receiving eculizumab and azathioprine died from pulmonary empyema (infectious pleural effusion), which the investigator categorized a probably related to trial treatment. Associated cultures yielded Streptococcus intermedius and Peptostreptococcus micros (which are not associated with complement deficiency). The patient had an extensive history of pulmonary disease and was an active smoker. No cases of meningococcal infection were reported. Rates per 100 patient-years of other serious infections were 8 events and 15 events with eculizumab and placebo, respectively.

Adverse events are summarized below in Table 31. Data exclude events of NMOSD that were relapses meeting the definition of a serious AE. The only serious AEs experienced by more than one patient in either group were pneumonia (three patients receiving eculizumab; one patient receiving placebo), and cellulitis, sepsis, and urinary tract infection (each experienced by two patients receiving eculizumab and none receiving placebo). Details of the death are reported in the Results section.

No cases of meningococcal infection were reported. The rates of other serious infections were 8.1 and 15.1 events per 100 patient-years with eculizumab and placebo, respectively.

No clinically significant trends were observed in other safety assessments.

TABLE 31
Summary of Adverse Events

| | Eculizumab (n = 96) | | | Placebo (n = 47) | | |
|---|---|---|---|---|---|---|
| | (173 patient-years) | | | (53 patient-years) | | |
| | | Rate | | | Rate | |
| | | (events per | | | (events per | |
| | | 100 patient- | | | 100 patient- | |
| | Event | years) | Patients no (%) | Event | years) | Patients no (%) |
| Any AE | 1288 | 745 | 88 (92) | 599 | 1127 | 43 (91) |
| Any AE related to | 366 | 212 | 49 (51) | 87 | 164 | 27 (57) |
| trial treatment | | | | | | |

Any AE according to severity

| | | | | | | |
|---|---|---|---|---|---|---|
| Severe | 27 | 16 | 15 (16) | 15 | 28 | 7 (15) |
| Moderate | 186 | 108 | 59 (61) | 143 | 269 | 25 (53) |
| Mild | 1072 | 620 | 86 (90) | 441 | 830 | 41 (87) |
| Unknown | 3 | 2 | 3 (3) | 0 | 0 | 0 |
| Any serious AE^{b} | 46 | 27 | 25 (26) | 29 | 55 | 13 (28) |
| Death^{c} | 1 | 1 | 1 (1) | 0 | 0 | 0 |
| Related to trial | 13 | 8 | 9 (9) | 13 | 24 | 9 (19) |
| treatment | | | | | | |
| AEs leading | 0 | 0 | 0 | 3 | 6 | 2 (4) |
| to treatment | | | | | | |
| discontinuation | | | | | | |

AEs experienced by ≥15% of patients in either treatment group

| | | | | | | |
|---|---|---|---|---|---|---|
| URTI | 54 | 31 | 28 (29) | 10 | 19 | 6 (13) |
| Headache | 95 | 55 | 22 (23) | 20 | 38 | 11 (23) |
| Nasopharyngitis | 50 | 29 | 20 (21) | 15 | 28 | 9 (19) |
| Nausea | 30 | 17 | 16 (17) | 19 | 36 | 12 (26) |
| Diarrhea | 23 | 13 | 15 (16) | 19 | 36 | 7 (15) |
| Urinary tract | 45 | 26 | 13 (14) | 13 | 24 | 10 (21) |
| infection | | | | | | |
| Pain in extremity | 13 | 8 | 11 (11) | 11 | 21 | 10 (21) |
| Vomiting | 10 | 6 | 10 (10) | 10 | 19 | 8 (17) |

An overview of additional events during treatment were recorded and summarized in Table 32 below.

TABLE 32
Overview of Additional Events for Key Safety Analysis

| | Placebo (N = 47) | | | Eculizumab (N = 96) | | |
|---|---|---|---|---|---|---|
| | Pt-years (PY) = 53.1 | | | Pt-years (PY) = 172.8 | | |
| | | Rate per | Patients | | Rate per | Patients |
| | Events n | 100 PY | n (%) | Events n | 100 PY | n (%) |
| Upper respiratory tract infection | 10 | 18.8 | 6 (12.8) | 54 | 31.2 | 28 (29.2) |
| Headache | 20 | 37.6 | 11 (23.4) | 95 | 55.0 | 22 (22.9) |
| Nasopharyngitis | 15 | 28.2 | 9 (19.1) | 50 | 28.9 | 20 (20.8) |
| Nausea | 19 | 35.7 | 12 (25.5) | 30 | 17.4 | 16 (16.7) |
| Urinary tract infection | 13 | 24.5 | 10 (21.3) | 45 | 26.0 | 13 (13.5) |
| Diarrhoea | 19 | 35.7 | 7 (14.9) | 23 | 13.3 | 15 (15.6) |
| Pain in extremity | 11 | 20.7 | 10 (21.3) | 13 | 7.5 | 11 (11.5) |
| Back pain | 9 | 16.9 | 6 (12.8) | 17 | 9.8 | 14 (14.6) |
| Dizziness | 6 | 11.3 | 6 (12.8) | 19 | 11.0 | 14 (14.6) |
| Cough | 9 | 16.9 | 7 (14.9) | 13 | 7.5 | 11 (11.5) |
| Vomiting | 10 | 18.8 | 8 (17.0) | 10 | 5.8 | 10 (10.4) |
| Arthralgia | 10 | 18.8 | 5 (10.6) | 12 | 6.9 | 11 (11.5) |
| Influenza | 2 | 3.8 | 2 (4.3) | 19 | 11.0 | 11 (11.5) |
| Pharyngitis | 3 | 5.6 | 3 (6.4) | 13 | 7.5 | 10 (10.4) |
| Confusion | 8 | 15.1 | 2 (4.3) | 11 | 6.4 | 10 (10.4) |

Serious treatment-emergent adverse events considered to be related to study treatment by the investigator is summarized in table 33 below.

TABLE 33
Serious Treatment-emergent Adverse Events Considered
to be Related to Study Treatment by the Investigator.

| | Eculizumab (N = 96) | Placebo (N = 47) |
|---|---|---|
| Patients with a serious | 9 (9.4) | 9 (19.1) |
| adverse event | | |
| Pneumonia | 2 (2.1) | 1 (2.1) |
| Bronchitis | 1 (1.0) | 1 (2.1) |
| Cellulitis | 1 (1.0) | 0 |
| Abdominal pain upper | 1 (1.0) | 0 |
| Atrial fibrillation | 1 (1.0) | 0 |
| Cholecystitis acute | 1 (1.0) | 0 |
| Gallbladder empyema | 1 (1.0) | 0 |
| Infectious pleural effusion | 1 (1.0)* | 0 |
| Pyrexia | 1 (1.0) | 0 |
| Renal abscess | 1 (1.0) | 0 |
| Sepsis | 1 (1.0) | 0 |
| Venous occlusion | 1 (1.0) | 0 |
| Abdominal pain | 0 | 1 (2.1) |
| Herpes zoster | 0 | 1 (2.1) |
| Influenza | 0 | 1 (2.1) |
| Pancytopenia | 0 | 1 (2.1) |
| Pleurisy | 0 | 1 (2.1) |
| Pneumococcal infection | 0 | 1 (2.1) |
| Pulmonary embolism | 0 | 1 (2.1) |
| Respiratory disorder | 0 | 1 (2.1) |
| Thromocytopenia | 0 | 1 (2.1) |
| Viral upper respiratory | 0 | 1 (2.1) |
| tract infection | | |

### 7.5.1. Significance Levels

For all analyses, the eculizumab treated group is compared to the placebo group and all hypothesis testing is two-sided and performed at the 0.05 level of significance, unless otherwise specified. Estimates of treatment effect on efficacy parameters will be accompanied by two-sided 95% confidence intervals for the effect size.

### 7.5.2. Missing or Invalid Data

For secondary and tertiary efficacy analyses, missing post-Baseline efficacy and safety data will not be imputed unless indicated in the described analysis in the SAP.

### 7.5.3. Summary

The discovery of the NMOSD AQP4-IgG biomarker and identification of complement as a major mechanism of injury ultimately led to the PREVENT study, a randomized, double-blind, placebo-controlled trial of eculizumab, a terminal complement inhibitor in AQP4-IgG-positive NMOSD. As progression independently of relapses is rare in NMOSD, the therapeutic imperative is to preserve neurologic function by preventing relapses. Eculizumab significantly reduced relapse risk compared with placebo in patients with AQP4-IgG+NMOSD.

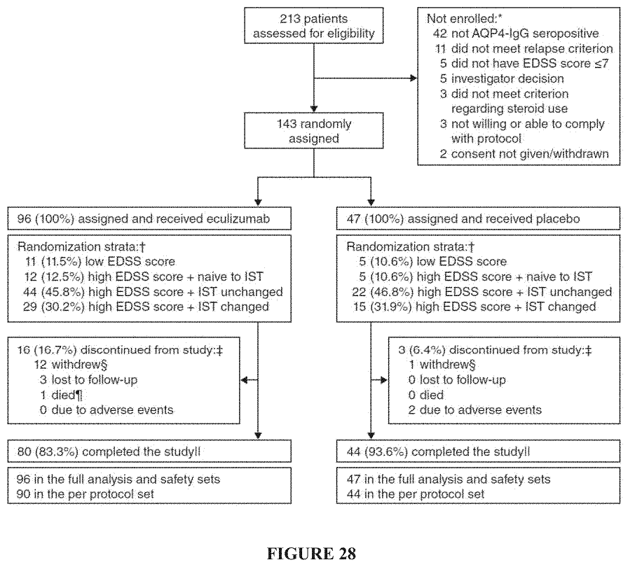

The scale and robust design of the PREVENT study set it apart from previous published research investigating relapse prevention to date. Patients were considered to have completed the study if the treating physician determined that they had experienced a relapse or if the study was ended because 23 positively adjudicated relapses had occurred in 23 different patients. Of 80 patients (83%) completing the trial in the eculizumab group, the majority (65 patients) completed because the trial ended (vs 15 patients because of a physician-determined relapse). Of 44 patients (94%) completing the trial in the placebo group, the majority (29 patients) completed because of a physician-determined relapse (vs 15 patients because the trial ended) (FIG. 28).

A greater number of patients withdrew from eculizumab than placebo treatment. However, even when allowing for differences in on-study time and the 2:1 randomization ratio, these withdrawals were largely related to changed patient circumstances. The reasons for withdrawal (§) in the eculizumab group included: moving away from the study site (four patients); unwilling to continue participating in a clinical study (two patients); economic reasons (one patient); employment (one patient); ongoing adverse event and difficult venous access (one patient). The reasons why the three remaining patients withdrew were not known. The patient withdrawing from the placebo group was unwilling to continue receiving investigational treatment. The time in the study for patients withdrawing from the eculizumab group ranged from 106 to 835 days. Corresponding discontinuation rates (‡), allowing for the longer time that patients in the eculizumab group spent in the study compared with those in the placebo group (i.e., 172.8 and 53.1 patient-years, respectively), were 9.3 per 100 patient-years and 5.6 per 100 patient-years.

Treatment with eculizumab reduced the proportion of patients experiencing a relapse across all subgroups; the proportion in the eculizumab and placebo groups overall was 3/96 (3.1%) and 20/47 (42.6%), respectively. EDSS scores were up to 2.0, and high scores were between 2.5 and 7.0 inclusive. IST was considered unchanged if no IST was started and/or discontinued, although doses may have changed, after the last relapse before screening. IST naïve refers to patients receiving no IST therapy, except steroids alone, previously. In the study, eculizumab significantly reduced the risk of relapse irrespective of the use of supportive IST. The treatment effect among patients not receiving concomitant supportive ISTs was consistent with that in the overall population.

Compared with the treatment effect on relapses, treatment effects on neurologic function, disability, and health-related quality of life were less substantial in the PREVENT study. The modest effect was expected because disability accumulates over multiple relapses, and the study design precluded follow-up beyond weeks after a single relapse. Although median changes from baseline to study end were zero, the distribution of changes overall indicated that patients receiving eculizumab tended to show greater improvements and those receiving placebo greater deteriorations.

By blocking the complement system, eculizumab increases the risk of infection with encapsulated bacteria. In the PREVENT study, all patients received a meningococcal vaccination and no cases of meningococcal infection were reported; there were also no safety signals related to other serious infections. One patient died (¶) from infectious pulmonary empyema, with associated cultures yielding Streptococcus intermedius and Peptostreptococcus. The investigator considered that the pulmonary empyema was probably related to study treatment. The microorganisms implicated in the case of pulmonary empyema are not associated with complement deficiency. The event was accompanied by pneumonia and sepsis that were considered unrelated to eculizumab, and significant underlying pulmonary disease had predisposed the patient to develop pneumonia.

Overall, the eculizumab safety profile was consistent with those in established indications.

### Example 2. Extension Trial

An extension trial is described herein that is run to evaluate the long-term safety of eculizumab in subjects with relapsing NMO. Other secondary objectives include:
Evaluate the long-term efficacy as measured by ARR
Evaluate long-term efficacy by additional efficacy measures including:
   Disability
   Quality of life
   Neurologic functions
Describe the PK and PD of eculizumab in relapsing NMO patients.

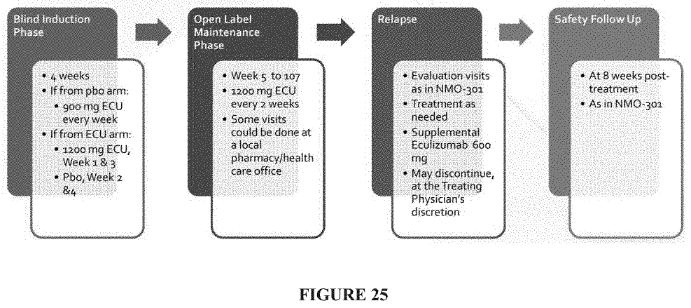

The extension trial lasts for 4 years (FPFV to LPLV). To maintain the blind of the previous trial, all subjects undergo a blind induction phase, followed by an open label maintenance phase. This is summarized in FIG. 25. Assessments, Treatment, Concomitant/Prohibited medications are performed as in the study described above.

The inclusion criteria for the extension trial is completion of the previous trial either through a relapse or completing the trial without relapse but completing EOS assessments. Exclusion criteria are withdrawing from the previous trial as a result of an AE related trial drug and pregnancy or intention to get pregnant. IST treatment can be changed at the treating physician's discretion but rituximab is prohibited. Subjects can continue with the trial if the experience relapse based on the Treating Physician's discretion or may transition to a commercial drug.

Efficacy is measured by change from baseline in ARR, EDSS, EQ-5D, mRS, HAI in patients with abnormal baseline ambulatory function, visual acuity in patients with abnormal baseline visual function, VA, SF-36 and EDSS FSS.

39 patients with a physician-determined relapse during PREVENT had entered by May 31, 2018, and were evaluated at this cut-off date. Patients received intravenous eculizumab (1200 mg/2 weeks after the induction period); stable-dose supportive immunosuppressive therapy (IST) permitted in PREVENT could be modified at the investigator's discretion during the OLE. The primary objective was safety and the primary efficacy endpoint was physician-determined annualized relapse rate (ARR) compared with the 24 months before PREVENT screening.

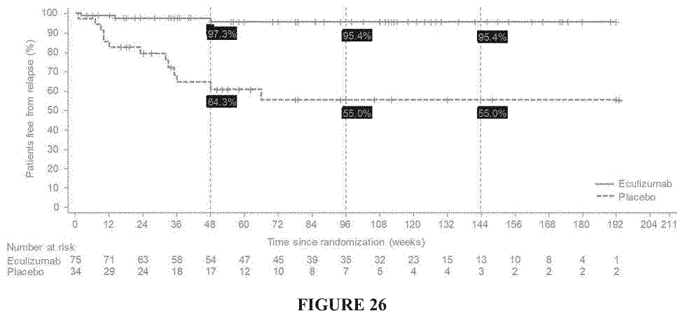

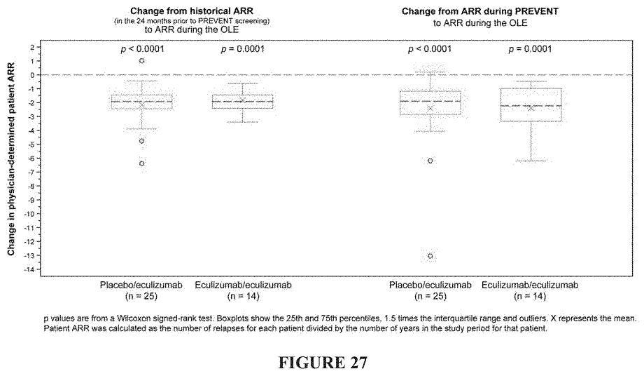

Median OLE duration was 85.14 weeks (range 6.4 176.6; 64.3 patient-years). Most adverse events (AEs) were mild-to-moderate (table). Overall, 9 serious infections occurred in 7 patients (most commonly in the urinary tract, 4 events). One patient discontinued treatment due to Sjogren's syndrome, autoimmune thyroiditis, and worsening systemic lupus erythematos. No meningococcal infections or deaths occurred in the OLE. Overall, 4 physician-determined relapses occurred in 4/14 (28.6%) patients receiving eculizumab/eculizumab and 9 in 4/25 (16%) receiving placebo/eculizumab, of which 3/4 and 1/9 were adjudicated positively by an independent, blinded expert committee (FIG. 27). Relapse rates were reduced in patients continuing eculizumab and in those switching from placebo (FIG. 26), and overall (primary efficacy endpoint; OLE median ARR 0 [range 0 2.46], historical 1.923 [0.96 6.38], p<0.0001). The adverse and serious adverse effects are summarized in table 34 below.

TABLE 34
Adverse Events (AEs) and Serious Adverse Events (SAEs)

| | Eculizumab (n = 39), 64.3 on-study patient-years | | |
|---|---|---|---|
| | | Rate | Patients, |
| | Events, n | (events/100 patient-years) | n (%) |
| AEs | 569 | 885.4 | 36 (92.3) |
| Related AEs | 122 | 189.8 | 23 (59.0) |
| SAEs | 36 | 56.0 | 13 (33.3) |
| Related SAEs | 9 | 14.0 | 5 (12.8) |

This data indicates that open-label eculizumab was well-tolerated. Reductions in relapse rates indicated benefit for patients continuing eculizumab or switching from placebo.

119 patients with a physician-determined relapse during PREVENT had entered the OLE by Oct. 31, 2018, and were evaluated at this cut-off date. Patients from PREVENT entered the OLE based on their physician-determined relapse status and received eculizumab (maintenance dose 1200 mg/2 weeks). Stable-dose immunosuppressive therapies used in PREVENT could be modified at the investigator's discretion. The primary objective was safety, and an interim analysis of time to relapse as adjudicated by independent panel vs pre-study rates (during 24 months prior to study) was also performed.

The mean pre-study physician-determined annualized relapse rate was 2.01 (SD 0.993), with a median (range) of 1.923 (0.96-6.38). Patients in the OLE were assessed for a median of 20.29 (5.1-198.4) weeks. The safety population comprised all patients who received eculizumab in PREVENT and/or the OLE (n=137; 282.3 patient-years [PY] of study duration), with a median total study duration of 107.86 (5.1-237.9) weeks. The rates of AEs and serious AEs per 100 PY were 763.1 and 37.6, respectively. The AE profile was similar in PREVENT and the OLE with no increase in infections observed over time. There was one death during PREVENT (pulmonary empyema) and none during the OLE. One patient reported meningococcal infection in the OLE. In the effectiveness analysis of 119 patients in the OLE, there were 15 physician-reported relapses, and the adjudicator-reported estimated proportion of patients who were relapse-free at week 192 was 93.9% (95% CI 87.5, 97.1). In this long-term safety and effectiveness analysis, eculizumab was well tolerated and the AEs reported were consistent with its established safety profile in other indications. The high proportion of relapse-free patients observed in PREVENT were maintained over 4 years.

### Example 3. Neuromyelitis Optica: Annual Relapse Rates Off and on Immunosuppression and the Relationship to Attack Type and Ethnicity

Data from 82 AQP4 antibody positive patients clinically assessed within the nationally commissioned NMO service in Oxford were analyzed.

Median onset age was 39 years, median follow up was 6 years, and 85% of subjects were female. The cohort included 45 Caucasians, 15 Afro-Caribbeans, 9 Asians and 13 mixed race/other. Only 3.6% had both transverse myelitis (TM) and optic neuritis at onset. The median time from onset to diagnosis had reduced from 12.4 years pre-2004 to 0.1 years post 2009 (when awareness of NMO was higher and the Oxford National clinical and assay service were established).

The lowest RRs were seen in Asian patients (44% with the lowest RR quartile) and the highest in the Afro-Caribbean patients (67% had the highest RR quartile). The latter may be related to a higher rate of relapses in brain/brainstem attacks. Younger patients (<18 yrs) had higher RR than those over 18 years: 1.53 versus 0.82.

The ‘pre-all-treatment’ RR was 0.87 versus an on-immunosuppressive treatment RR of 0.42. However delaying treatment did not appear to affect the on-treatment RR. Annual RRs for 13 patients untreated for ≥4 years were: yr. 1: 1.46 (including onset attack), yr. 2: 0.23, yr. 3: 0.15 and yr. 4: 0.15 (probably biased by milder patients being more likely to stay off treatment). The pre-all-treatment RR compared to the ‘on’ individual treatment rates (±prednisolone) were: azathioprine (n=66, 63 1st line) 0.93:0.21, methotrexate (n=16, 6 1st line) 1.9:0.44, mycophenolate mofetil (n=17, 4 1st line) 0.76:0.50, rituximab (n=10, 1 1st line) 0.76:0.46.

Immunosuppressive treatment appeared to reduce the residual disability caused by attacks of TM: Mean downstream minus pre-relapse EDMUS scores and % with no residual change in EDMUS were pre-treatment+0.8, 54% vs-0.05, 88%.

Immunosuppressive therapy is the ‘current standard of care’ to prevent relapses. Immunosuppressive therapy seems to reduce the residual damage from relapses. This data suggests that relapse frequency is influenced by ethnicity, onset age, and attack type.

TABLE 35
List of Abbreviations and Definitions of Terms

| Abbreviation or | |
|---|---|
| Specialist Term | Explanation |
| AE | Adverse event |
| aHUS | Atypical hemolytic uremic syndrome |
| AQP4 | Aquaporin- 4 |
| AQP4-Ab | Aquaporin- 4 antibody |
| AMR | Antibody Mediated Rejection |
| AZA | Azathioprine |
| BBB | Blood brain barrier |
| BP | Blood Pressure |
| C5 | Complement protein 5 |
| CM | Centimeter |
| CMAX | Maximal concentration |
| CMIN | Minimal concentration |
| CNS | Central Nervous System |
| CRF | Case Report Form |
| CSF | Cerebrospinal fluid |
| C-SSRS | Columbia-Suicide Severity Rating Scale |
| DMC | Data Monitoring Committee |
| ECG | Electrocardiogram |
| eDC | Electronic Data Capture |
| EDSS | Expanded Disability Status Scale |
| EIU | Exposure in-utero |
| EOS | End of Study Period |
| EQ-5D | EuroQoL |
| ET | Early Termination |
| EU | Europe |
| FAS | Full Analysis Set |
| FS | Functional System |
| FSS | Functional System Scores |
| GCP | Good Clinical Practice |
| HAHA | Human Anti-human Antibody |
| HAI | Hauser Ambulation Index |
| HCG | Human Chorionic gGonadotropin |
| HR | Heart Rate |
| HRL | Historical relapse |
| IB | Investigators Brochure |
| ICF | Informed Consent Form |
| ICH | International Conference on Harmonization |
| ICU | Intensive Care Unit |
| IEC | Independent Ethics Committee |
| IgG | Immunoglobulin G |
| IIT | Investigator-Initiated Trial |
| IP | Investigational Product |
| IRB | Institutional Review Board |
| IST | Immunosuppressant Therapy |
| IV | Intravenous |
| IVIg | Intravenous Immunoglobulin |
| IVMP | Intravenous Methylprednisolone |
| IXRS | Interactive voice or web Response System |
| KGS | Kilograms |
| LBS | Pounds |
| LETM | Longitudinally Extensive Transverse Myelitis |
| mAb | Monoclonal Antibody |
| MMF | Mycophenolate Mofetil |
| mRS | Modified Rankin Scale |
| MRI | Magnetic Resonance Imaging |
| MS | Multiple Sclerosis |
| NMO | Neuromyelitis Optica |
| NMO-IgG | Neuromyelitis Optica antibody |
| NMOSD | Neuromyelitis Optica Spectrum Disorders |
| ON | Optic Neuritis |
| OSIS | Optic Spinal Impairment Score |
| PD | Pharmacodynamics |
| PE | Plasmapheresis or Plasma Exchange |
| PI | Principal Investigator |
| PK | Pharmacokinetics |
| PNH | Paroxysmal Nocturnal Hemoglobinuria |
| PP | Per-Protocol Population |
| QOL | Quality of Life |
| RR | Respiration Rate |
| SAE | Serious Adverse Event |
| SAP | Statistical Analysis Plan |
| SF-36 | Short Form Health Survey |
| SOC | System Organ Class |
| TEAE | Treatment Emergent Adverse Events |
| VA | Visual Acuity |
| VS | Vital Signs |
| US | United States |

TABLE 36
Additional Characteristics at Baseline.

| Characteristic | Eculizumab (N = 96) | | Placebo (N = 47) | | Total (N = 143) | |
|---|---|---|---|---|---|---|
| Supportive immunosuppressive therapy used before the study | 88 | (91.7) | 45 | (95.7) | 133 | (93.0) |
| Corticosteroids | 68 | (70.8) | 30 | (63.8) | 98 | (68.5) |
| Azathioprine | 61 | (63.5) | 26 | (55.3) | 87 | (60.8) |
| Mycophenolate mofetil | 27 | (28.1) | 15 | (31.9) | 42 | (29.4) |
| Rituximab | 26 | (27.1) | 20 | (42.6) | 46 | (32.2) |
| Cyclophosphamide | 8 | (8.3) | 5 | (10.6) | 13 | (9.1) |
| Methotrexate | 4 | (4.2) | 5 | (10.6) | 9 | (6.3) |
| Cyclosporine and tacrolimus | 3 | (3.1) | 3 | (6.4) | 6 | (4.2) |
| Mitoxantrone and cladribine | 3 | (3.1) | 3 | (6.4) | 6 | (4.2) |
| Intravenous immunoglobulin | 2 | (2.1) | 2 | (4.3) | 4 | (2.8) |
| Tocilizumab | 2 | (2.1) | 0 | | 2 | (1.4) |
| Mizoribine | 1 | (1.0) | 2 | (4.3) | 3 | (2.1) |
| History of an autoimmune disorder^{†} | 23 | (24.0) | 10 | (21.3) | 33 | (23.1) |
| Autoimmune thyroiditis | 6 | (6.3) | 1 | (2.1) | 7 | (4.9) |
| Rheumatoid arthritis | 5 | (5.2) | 2 | (4.3) | 7 | (4.9) |
| Systemic lupus erythematosus | 4 | (4.2) | 6 | (12.8) | 10 | (7.0) |
| Sjögren's syndrome | 4 | (4.2) | 3 | (6.4) | 7 | (4.9) |
| Myasthenia gravis | 2 | (2.1) | 0 | | 2 | (1.4) |
| Psoriasis | 1 | (1.0) | 1 | (2.1) | 2 | (1.4) |

| | | | | | |
|---|---|---|---|---|---|
| Autoimmune hemolytic anemia | 1 | (1.0) | 0 | 1 | (0.7) |
| Immune thrombocytopenic purpura | 1 | (1.0) | 0 | 1 | (0.7) |

| | | | | | |
|---|---|---|---|---|---|
| Lupus hepatitis | 0 | 1 | (2.1) | 1 | (0.7) |

### Example 4. Neuromyelitis Optica: Japanese Cohort

Fourteen Japanese patients meeting the inclusion criteria above were included in the study. Nine of those patients received eculizumab and five received placebo. Thirteen of the patients completed the trial. One patient in the placebo group discontinued owing to pancytopenia and pre-renal failure. The baseline characteristics of the Japanese cohort is listed in Table 37 below. Overall, baseline characteristics were similar for Japanese and non-Japanese patients.

TABLE 37
Baseline characteristics of Japanese cohort

| Characteristic | Eculizumab (n = 9) | | Placebo (n = 5) | |
|---|---|---|---|---|
| Female sex, n (%) | 8 | (89) | 4 | (80) |
| Age at first dose of trial | 46.8 | (14.23) | 51.0 | (10.12) |
| medication, mean (SD), years | | | | |
| Diagnosis of NMO/NMOSD, | 5 (56)/4 (44) | | 3 (60)/2 (40) | |
| n (%) | | | | |
| ARR over previous 24 months, | 2.19 | (1.14) | 2.02 | (0.93) |
| mean (SD) | | | | |
| EDSS scores, median (range) | 4.00 | (3.0-7.0) | 6.00 | (3.5-6.0) |
| ISTs at baseline, n (%) | 0 | | 0 | |
| None | | | | |
| Glucocorticoids alone | 3 | (33) | 2 | (40) |
| Azathioprine ± glucocorticoids | 3 | (33) | 2 | (40) |
| Mycophenolate mofetil ± | 0 | | 0 | |
| glucocorticoids | | | | |
| Other IST ± glucocorticoids | 3 | (33) | 1 | (20) |

| | | |
|---|---|---|
| Previous rituximab treatment, | 0 | 0 |
| n (%) | | |

Unadjusted ARRs (95% CI) in the Japanese cohorts were: 0.06 (0.01, 0.45) with eculizumab and 0.48 (0.12, 1.93) with placebo, as listed in Table 38 below. As expected, average scores on scales of disability and quality of life were minimally altered. There was no evidence showing a difference between Japanese and global cohorts in the reduction of risk of adjudicated relapse and changes in disability and quality of life.

TABLE 38
Japanese cohort: efficacy

| | Eculizumab (n = 9) | | Placebo (n = 5) | |
|---|---|---|---|---|
| Patients with adjudicated | 1 | (11) | 2 | (40) |
| relapse, n (%) | | | | |
| Follow-up time in weeks, median | 108 | (14-190) | 34 | (8-96) |
| (min-max) | | | | |

Estimated proportion of patients relapse-free (%)

| | | |
|---|---|---|
| 48 weeks | 88.9 | 60.0 |
| 96 weeks | 88.9 | 60.0 |
| 144 weeks | 88.9 | N/A |

The overall safety results for Japanese cohort are listed in Table 39 below. Treatment-related AEs for the Japanese cohort included AEs categorized as unlikely to be related to treatment in addition to the categories used for the global cohort (possibly, probably or definitely related to treatment, and AEs with an unknown relationship). The overall safety results for the Japanese cohort were consistent with those for the global cohort.

TABLE 39
Overall Safety Results for Japanese Cohort

| Eculizumab (n = 9) (16 PY) | | Placebo (n = 5) (4 PY) | |
|---|---|---|---|
| Events/100 PY | Patients, n (%) | Events/100 PY | Patients, n (%) |

AEs excluding NMOSD relapses meeting definition for serious AE

| Any AE | 675 | 9 | (100) | 823 | 4 (80) |
|---|---|---|---|---|---|
| Severe | 0 | 0 | (0) | 24 | 1 (20) |
| Moderate | 69 | 6 | (67) | 235 | 3 (60) |
| Mild | 606 | 9 | (100) | 564 | 4 (80) |
| Any treatment-related | 316 | 9 | (100) | 517 | 3 (60) |
| AE^{a} | | | | | |
| Any serious AE | 50 | 4 | (44) | 24 | 1 (20) |
| Death | 0 | 0 | | 0 | 0 |
| Related to treatment^{a} | 38 | 3 | (33) | 24 | 1 (20) |

| | | | | |
|---|---|---|---|---|
| AEs leading to | 0 | 0 | 47 | 1 (20) |
| discontinuation of trial | | | | |
| agent | | | | |

In summary, treatment effect in the Japanese cohort was consistent with that in the global cohort. There were no safety concerns specific to the Japanese cohort.

TABLE 40
List of Amino Acid Sequences

SEQ ID NO: 1
GYIFSNYWIQ
SEQ ID NO: 2
EILPGSGSTEYTENFKD
SEQ ID NO: 3
YFFGSSPNWYFDV
SEQ ID NO: 4
GASENIYGALN
SEQ ID NO: 5
GATNLAD
SEQ ID NO: 6
QNVLNTPLT
SEQ ID NO: 7
QVQLVQSGAE VKKPGASVKV SCKASGYIFS NYWIQWVRQA
PGQGLEWMGE ILPGSGSTEY TENFKDRVTM TRDTSTSTVY
MELSSLRSED TAVYYCARYF FGSSPNWYFD VWGQGTLVTV
SS
SEQ ID NO: 8
DIQMTQSPSS LSASVGDRVT ITCGASENIY GALNWYQQKP
GKAPKLLIYG ATNLADGVPS RFSGSGSGTD FTLTISSLQP
EDFATYYCQN VLNTPLTFGQ GTKVEIK
SEQ ID NO: 9
QVQLVQSGAE VKKPGASVKV SCKASGYIFS NYWIQWVRQA
PGQGLEWMGE ILPGSGSTEY TENFKDRVTM TRDTSTSTVY
MELSSLRSED TAVYYCARYF FGSSPNWYFD VWGQGTLVTV
SSASTKGPSV FPLAPCSRST SESTAALGCL VKDYFPEPVT
VSWNSGALTS GVHTFPAVLQ SSGLYSLSSV VTVPSSNFGT
QTYTCNVDHK PSNTKVDKTV ERKCCVECPP CPAPPVAGPS
VFLFPPKPKD TLMISRTPEV TCVVVDVSQE DPEVQFNWYV
DGVEVHNAKT KPREEQFNST YRVVSVLTVL HQDWLNGKEY
KCKVSNKGLP SSIEKTISKA KGQPREPQVY TLPPSQEEMT
NQVSLTCLVK GFYPSDIAVE WESNGQPENN YKTTPPVLDS
DGSFFLYSRL TVDKSRWQEG NVFSCSVMHE ALHNHYTQKS
LSLSLGK
SEQ ID NO: 10
DIQMTQSPSS LSASVGDRVT ITCGASENIY GALNWYQQKP
GKAPKLLIYG ATNLADGVPS RFSGSGSGTD FTLTISSLQP
EDFATYYCQN VLNTPLTFGQ GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC
SEQ ID NO: 11
SYAIS
SEQ ID NO: 12
GIGPFFGTANYAQKFQG
SEQ ID NO: 13
DTPYFDY
SEQ ID NO: 14
SGDSIPNYYVY
SEQ ID NO: 15
DDSNRPS
SEQ ID NO: 16
QSFDSSLNAEV
SEQ ID NO: 17
QVQLVQSGAE VKKPGSSVKV SCKASGGTFS SYAISVWRQA
PGQGLEWMGG IGPFFGTANY AQKFQGRVTI TADESTSTAY
MELSSLRSED TAVYYCARDT PYFDYWGQGT LVTVSS
SEQ ID NO: 18
DIELTQPPSV SVAPGQTARI SCSGDSIPNY YVYWYQQKPG
QAPVLVIYDD SNRPSGIPER FSGSNSGNTA TLTISGTQAE
DEADYYCQSF DSSLNAEVFG GGTKLTVL
SEQ ID NO: 19
NYIS
SEQ ID NO: 20
IIDPDDSYTEYSPSFQG
SEQ ID NO: 21
YEYGGFDI
SEQ ID NO: 22
SGDNIGNSYVH
SEQ ID NO: 23
KDNDRPS
SEQ ID NO: 24
GTYDIESYV
SEQ ID NO: 25
EVQLVQSGAE VKKPGESLKI SCKGSGYSFT NYISWVRQMP
GKGLEWMGII DPDDSYTEYS PSFQGQVTIS ADKSISTAYL
QWSSLKASDT AMYYCARYEY GGFDIWGQGT LVTVSS
SEQ ID NO: 26
SYELTQPPSV SVAPGQTARI SCSGDNIGNS YVHWYQQKPG
QAPVLVIYKD NDRPSGIPER FSGSNSGNTA TLTISGTQAE
DEADYYCGTY DIESYVFGGG TKLTVL
SEQ ID NO: 27
SSYYVA
SEQ ID NO: 28
AIYTGSGATYKASWAKG
SEQ ID NO: 29
DGGYDYPTHAMHY
SEQ ID NO: 30
QASQNIGSSLA
SEQ ID NO: 31
GASKTHS
SEQ ID NO: 32
QSTKVGSSYGNH
SEQ ID NO: 33
QVQLVESGGG LVQPGGSLRL SCAASGFTSH SSYYVAWVRQ
APGKGLEWVG AIYTGSGATY KASWAKGRFT ISKDTSKNQV
VLTMTNMDPV DTATYYCASD GGYDYPTHAM HYWGQGTLVT
VSS
SEQ ID NO: 34
DVVMTQSPSS LSASVGDRVT ITCQASQNIG SSLAWYQQKP
GQAPRLLIYG ASKTHSGVPS RFSGSGSGTD FTLTISSLQP
EDVATYYCQS TKVGSSYGNH FGGGTKVEIK
SEQ ID NO: 35
QVQLVESGGG LVQPGRSLRL SCAASGFTVH SSYYMAWVRQ
APGKGLEWVG AIFTGSGAEY KAEWAKGRVT ISKDTSKNQV
VLTMTNMDPV DTATYYCASD AGYDYPTHAM HYWGQGTLVT
VSSASTKGPS VFPLAPSSKS TSGGTAALGC LVKDYFPEPV
TVSWNSGALT SGVHTFPAVL QSSGLYSLSS VVTVPSSSLG
TQTYICNVNH KPSNTKVDKK VEPKSCDKTH TCPPCPAPEL
RRGPKVFLFP PKPKDTLMIS RTPEVTCVVV DVSHEDPEVK
FNWYVDGVEV HNAKTKPREE QYNSTYRVVS VLTVLHQDWL
NGKEYKCKVS NKGLPSSIEK TISKAKGQPR EPQVYTLPPS
REEMTKNQVS LTCLVKGFYP SDIAVEWESN GQPENNYKTT
PPVLDSDGSF FLYSKLTVDK SRWQQGNVFS CSVLHEALHA
HYTRKELSLS P
SEQ ID NO: 36
DIQMTQSPSS LSASVGDRVT ITCRASQGIS SSLAWYQQKP
GKAPKLLIYG ASETESGVPS RFSGSGSGTD FTLTISSLQP
EDFATYYCQN TKVGSSYGNT FGGGTKVEIK RTVAAPSVFI
FPPSDEQLKS GTASVVCLLN NFYPREAKVQ WKVDNALQSG
NSQESVTEQD SKDSTYSLSS TLTLSKADYE KHKVYACEVT
HQGLSSPVTK SFNRGEC
SEQ ID NO: 37
QVQLQESGPG LVKPSETLSL TCTVSGDSVS SSYWTWIRQP
PGKGLEWIGY IYYSGSSNYN PSLKSRATIS VDTSKNQFSL
KLSSVTAADT AVYYCAREGN VDTTMIFDYW GQGTLVTVSS
SEQ ID NO: 38
AIQMTQSPSS LSASVGDRVT ITCRASQGIR NDLGWYQQKP
GKAPKLLIYA ASSLQSGVPS RFAGRGSGTD FTLTISSLQP
EDFATYYCLQ DFNYPWTFGQ GTKVEIK
SEQ ID NO: 39
QVQLQESGPG LVKPSETLSL TCTVSGDSVS SSYWTWIRQP
PGKGLEWIGY IYYSGSSNYN PSLKSRATIS VDTSKNQFSL
KLSSVTAADT AVYYCAREGN VDTTMIFDYW GQGTLVTVSS
ASTKGPSVFP LAPCSRSTSE STAALGCLVK DYFPEPVTVS
WNSGALTSGV HTFPAVLQSS GLYSLSSVVT VPSSSLGTKT
YTCNVDHKPS NTKVDKRVES KYGPPCPPCP APEFLGGPSV
FLFPPKPKDT LMISRTPEVT CVVVDVSQED PEVQFNWYVD
GVEVHNAKTK PREEQFNSTY RVVSVLTVLH QDWLNGKEYK
CKVSNKGLPS SIEKTISKAK GQPREPQVYT LPPSQEEMTK
NQVSLTCLVK GFYPSDIAVE WESNGQPENN YKTTPPVLDS
DGSFFLYSRL TVDKSRWQEG NVFSCSVMHE ALHNHYTQKS
LSLSLGK
SEQ ID NO: 40
AIQMTQSPSSL SASVGDRVTI TCRASQGIRN DLGWYQQKPGK
APKLLIYAAS SLQSGVPSRFA GRGSGTDFTL TISSLQPEDF
ATYYCLQDFNY PWTFGQGTKV EIKRTVAAPSV FIFPPSDEQL
KSGTASVVCL LNNFYPREAKV QWKVDNALQS GNSQESVTEQD
SKDSTYSLSS TLTLSKADYE KHKVYACEVTH QGLSSPVTKS
FNRGEC

1. A method of treating a neuromyelitis optica spectrum disorder (NMOSD) in a human subject in need thereof comprising administering a therapeutically effective amount of eculizumab to the human subject, wherein the human subject is positive for auto-antibodies binding aquaporin-4 (NMO-IgG) and shows one or more signs or symptoms of NMSOD.

2. The method of claim 1, wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28.

3. The method of claim 2, wherein the 28 day induction phase of eculizumab treatment is followed by a maintenance phase comprising administering 1200 mg of eculizumab 14 days after the fifth induction dose and administering 1200 mg of eculizumab every 14±2 days thereafter.

4. The method of claim 3, further comprising performing plasmapheresis on the human subject and administering eculizumab at a dose of between 300 mg and 1200 mg to the human subject within 4 hours of completion of plasmapheresis.

5. The method of claim 3, further comprising performing plasmapheresis on the human subject and administering eculizumab at a dose of between 600 mg and 900 mg to the human subject within 90 minutes of completion of plasmapheresis.

6. The method of claim 3, further comprising performing plasmapheresis on the human subject and administering eculizumab at a dose of 600 mg to the human subject within 1 hour of completion of plasmapheresis.

7. The method of claim 1, wherein before administration of eculizumab, the human subject experienced three or more relapses in the previous 24 months or at least two relapses in the previous 12 months, wherein the human subject having three or more relapses in the previous 24 months experienced at least one relapse in the previous 12 months.

8. The method of claim 1, wherein before administration of eculizumab, the human subject experienced an annualized relapse rate (ARR) of greater than 1.0 in the previous 24 months.

9. The method of claim 1, wherein before administration of eculizumab, the human subject experienced an Expanded Disability Status Scale (EDSS) score of greater than 1.0 in the previous 24 months.

10. The method of claim 9, wherein the human subject experienced an EDSS score greater than 2.5 and less than 7 in the 24 months before administration of eculizumab.

11. The method of claim 1, wherein before administration of eculizumab, the human subject experienced a Hauser ambulation index (HAI) score of 0.0 or greater in the previous 24 months.

12. The method of claim 1, wherein before administration of eculizumab, the human subject experienced a modified Rankin Scale (mRS) score of 0.0 to 2.5 in the previous 24 months.

13. The method of claim 1, wherein eculizumab is administered without additional immunosuppressive therapies (ISTs).

14. The method of claim 1, wherein eculizumab is administered with at least one IST.

15. The method of claim 14, wherein the at least one IST is selected from the group consisting of a steroid, azathioprine (AZA), and mycophenolate mofetil (MMF).

16. The method of claim 15, wherein eculizumab is administered with a steroid and at least one additional IST.

17. The method of claim 16, wherein the at least one additional IST is AZA or MMF.

18. The method of claim 1, wherein the human subject experiences a clinically meaningful improvement in one or more clinical markers for NMOSD progression after administration of eculizumab.

19. The method of claim 18, wherein the one or more clinical markers for NMOSD progression are selected from the group consisting of ARR, EDSS, HAI, and mRS.

20. The method of claim 1, wherein the human subject experiences a greater than 80% risk reduction for relapse after administration of eculizumab.

20. The method of claim 1, wherein the human subject experiences a greater than 90% risk reduction for relapse after administration of eculizumab.

21. The method of claim 1, wherein the human subject has a time to relapse greater than 6 months after administration of eculizumab.

22. The method of claim 1, wherein the human subject has a time to relapse greater than 48 weeks after administration of eculizumab.

23. The method of claim 1, wherein the human subject has a time to relapse greater than 24 months after administration of eculizumab.

24. The method of claim 1, wherein eculizumab is administered by intravenous infusion.

25. The method of claim 1, wherein the human subject is 18 years of age or older.

26. The method of claim 1, wherein the human subject is administered eculizumab for at least 26 weeks.

27. The method of claim 1, further comprising selecting the human subject for displaying one or more symptoms of NMOSD prior to administration of eculizumab.

28. A method of treating a neuromyelitis optica spectrum disorder (NMOSD) in a human subject in need thereof comprising administering a therapeutically effective amount of eculizumab to the human subject;
wherein the human subject is positive for auto-antibodies binding aquaporin-4 (NMO-IgG) and shows one or more signs or symptoms of NMSOD;
wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28; and
wherein the human subject experiences a clinically meaningful improvement in one or more clinical markers for NMOSD progression after administration of eculizumab, wherein the one or more clinical markers for NMOSD progression are selected from the group consisting of ARR, EDSS, HAI, and mRS.

29. A method of treating a neuromyelitis optica spectrum disorder (NMOSD) in a human subject in need thereof comprising administering a therapeutically effective amount of eculizumab to the human subject;
wherein the human subject is positive for auto-antibodies binding aquaporin-4 (NMO-IgG) and shows one or more signs or symptoms of NMSOD;
wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28;
wherein the 28 day induction phase of eculizumab treatment is followed by a maintenance phase comprising administering 1200 mg of eculizumab 14 days after the fifth induction dose and administering 1200 mg of eculizumab every 14±2 days thereafter; and
wherein the human subject experiences a clinically meaningful improvement in one or more clinical markers for NMOSD progression after administration of eculizumab, wherein the one or more clinical markers for NMOSD progression are selected from the group consisting of ARR, EDSS, HAI, and mRS.

30. A method of treating a neuromyelitis optica spectrum disorder (NMOSD) in a human subject in need thereof comprising administering a therapeutically effective amount of eculizumab to the human subject;
wherein the human subject is positive for auto-antibodies binding aquaporin-4 (NMO-IgG) and shows one or more signs or symptoms of NMSOD;
wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28;
wherein the 28 day induction phase of eculizumab treatment is followed by a maintenance phase comprising administering 1200 mg of eculizumab 14 days after the fifth induction dose and administering 1200 mg of eculizumab every 14±2 days thereafter;
wherein plasmapheresis is performed on the human subject and administering eculizumab at a dose of 600 mg to the human subject within 1 hour of completion of plasmapheresis; and
wherein the human subject experiences a clinically meaningful improvement in one or more clinical markers for NMOSD progression after administration of eculizumab, wherein the one or more clinical markers for NMOSD progression are selected from the group consisting of ARR, EDSS, HAI, and mRS.


SEQUENCE LISTING (40 sequences)

SEQ ID NO: 1 - protein, 10 aa, Artificial Sequence
  Heavy chain CDR sequence 1
GYIFSNYWIQ

SEQ ID NO: 2 - protein, 17 aa, Artificial Sequence
  Heavy chain CDR sequence 2
EILPGSGSTEYTENFKD

SEQ ID NO: 3 - protein, 13 aa, Artificial Sequence
  Heavy chain CDR sequence 3
YFFGSSPNWYFDV

SEQ ID NO: 4 - protein, 11 aa, Artificial Sequence
  Light chain CDR sequence 1
GASENIYGALN

SEQ ID NO: 5 - protein, 7 aa, Artificial Sequence
  Light chain CDR sequence 2
GATNLAD

SEQ ID NO: 6 - protein, 9 aa, Artificial Sequence
  Light chain CDR sequence 3
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 447 aa, Artificial Sequence
  Heavy chain sequence
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, Artificial Sequence [sequence omitted]

SEQ ID NO: 11 - protein, 5 aa, Artificial Sequence
  Heavy chain CDR sequence 1
SYAIS

SEQ ID NO: 12 - protein, 17 aa, Artificial Sequence
  Heavy chain CDR sequence 2
GIGPFFGTANYAQKFQG

SEQ ID NO: 13 - protein, 7 aa, Artificial Sequence
  Heavy chain CDR sequence 3
DTPYFDY

SEQ ID NO: 14 - protein, 11 aa, Artificial Sequence
  Light chain CDR sequence 1
SGDSIPNYYVY

SEQ ID NO: 15 - protein, 7 aa, Artificial Sequence
  Light chain CDR sequence 2
DDSNRPS

SEQ ID NO: 16 - protein, 11 aa, Artificial Sequence
  Light chain CDR sequence 3
QSFDSSLNAEV

SEQ ID NO: 17 - protein, 116 aa, Artificial Sequence
  Heavy chain variable region sequence
QVQLVQSGAEVKKPGSSVKVSCKASGGTFSSYAISVWRQAPGQGLEWMGGIGPFFGTANYAQKFQGRVTITADESTSTAYMELSSLRSEDTAVYYCARDTPYFDYWGQGTLVTVSS

SEQ ID NO: 18 - protein, 108 aa, Artificial Sequence
  Light chain variable region sequence
DIELTQPPSVSVAPGQTARISCSGDSIPNYYVYWYQQKPGQAPVLVIYDDSNRPSGIPERFSGSNSGNTATLTISGTQAEDEADYYCQSFDSSLNAEVFGGGTKLTVL

SEQ ID NO: 19 - protein, 4 aa, Artificial Sequence
  Heavy chain CDR sequence 1
NYIS

SEQ ID NO: 20 - protein, 17 aa, Artificial Sequence
  Heavy chain CDR sequence 2
IIDPDDSYTEYSPSFQG

SEQ ID NO: 21 - protein, 8 aa, Artificial Sequence
  Heavy chain CDR sequence 3
YEYGGFDI

SEQ ID NO: 22 - protein, 11 aa, Artificial Sequence
  Light chain CDR sequence 1
SGDNIGNSYVH

SEQ ID NO: 23 - protein, 7 aa, Artificial Sequence
  Light chain CDR sequence 2
KDNDRPS

SEQ ID NO: 24 - protein, 9 aa, Artificial Sequence
  Light chain CDR sequence 3
GTYDIESYV

SEQ ID NO: 25 - protein, 116 aa, Artificial Sequence
  Heavy chain variable region sequence
EVQLVQSGAEVKKPGESLKISCKGSGYSFTNYISWVRQMPGKGLEWMGIIDPDDSYTEYSPSFQGQVTISADKSISTAYLQWSSLKASDTAMYYCARYEYGGFDIWGQGTLVTVSS

SEQ ID NO: 26 - protein, 106 aa, Artificial Sequence
  Light chain variable region sequence
SYELTQPPSVSVAPGQTARISCSGDNIGNSYVHWYQQKPGQAPVLVIYKDNDRPSGIPERFSGSNSGNTATLTISGTQAEDEADYYCGTYDIESYVFGGGTKLTVL

SEQ ID NO: 27 - protein, 6 aa, Artificial Sequence
  Heavy chain CDR sequence 1
SSYYVA

SEQ ID NO: 28 - protein, 17 aa, Artificial Sequence
  Heavy chain CDR sequence 2
AIYTGSGATYKASWAKG

SEQ ID NO: 29 - protein, 13 aa, Artificial Sequence
  Heavy chain CDR sequence 3
DGGYDYPTHAMHY

SEQ ID NO: 30 - protein, 11 aa, Artificial Sequence
  Light chain CDR sequence 1
QASQNIGSSLA

SEQ ID NO: 31 - protein, 7 aa, Artificial Sequence
  Light chain CDR sequence 2
GASKTHS

SEQ ID NO: 32 - protein, 12 aa, Artificial Sequence
  Light chain CDR sequence 3
QSTKVGSSYGNH

SEQ ID NO: 33 - protein, 123 aa, Artificial Sequence
  Heavy chain variable region sequence
QVQLVESGGGLVQPGGSLRLSCAASGFTSHSSYYVAWVRQAPGKGLEWVGAIYTGSGATYKASWAKGRFTISKDTSKNQVVLTMTNMDPVDTATYYCASDGGYDYPTHAMHYWGQGTLVTVSS

SEQ ID NO: 34 - protein, 110 aa, Artificial Sequence
  Light chain variable region sequence
DVVMTQSPSSLSASVGDRVTITCQASQNIGSSLAWYQQKPGQAPRLLIYGASKTHSGVPSRFSGSGSGTDFTLTISSLQPEDVATYYCQSTKVGSSYGNHFGGGTKVEIK

SEQ ID NO: 35 - protein, 451 aa, Artificial Sequence
  Heavy chain sequence
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 36 - protein, 217 aa, Artificial Sequence
  Light chain sequence
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 120 aa, Artificial Sequence
  Heavy chain variable region sequence
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 38 - protein, 107 aa, Artificial Sequence
  Light chain variable region sequence
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 39 - protein, 447 aa, Artificial Sequence
  Heavy chain sequence
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 214 aa, Artificial Sequence
  Light chain sequence
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC